(19) United States
(12) Patent Application Publication
(10) Pub. No.: US 2025/0143781 A1
(43) Pub. Date: May 8, 2025

(54) ENERGY DELIVERY SYSTEMS WITH LESION INDEX

(71) Applicant: Acutus Medical, Inc., Carlsbad, CA (US)

(72) Inventors: Ahmad FALAHATPISHEH, San Marcos, CA (US); Xinwei Shi, San Diego, CA (US); Shibaji Shome, San Diego, CA (US); Derrick Chou, San Diego, CA (US); Timothy Corvi, San Diego, CA (US); Alex Asconeguy, Eagle, ID (US); Steven Richard Mickelsen, Iowa City, IA (US); Ting Yang, Oceanside, CA (US); Wilson William Good, Carlsbad, CA (US); Eric Douglas Sauter, Carlsbad, CA (US); J. Christopher Flaherty, Nottingham, NH (US)

(21) Appl. No.: 18/291,334

(22) PCT Filed: Jul. 27, 2022
(86) PCT No.: PCT/US2022/038461

§ 371 Date: Jan. 23, 2024

Related U.S. Application Data
(60) Provisional application No. 63/226,040, filed on Jul. 27, 2021.
(60) Provisional application No. 63/336,245, filed on Apr. 28, 2022.

(51) Int. Cl. A61B 18/14; A61B 17/00; A61B 18/00

(52) U.S. Cl. CPC A61B 18/1492; A61B 2017/00199; A61B 2018/00357; A61B 2018/00577

(57) ABSTRACT

Provided herein are systems, devices, and methods for treating tissue of a patient. The system includes a first energy delivery device having a first energy delivery element to be positioned proximate target tissue of the patient, a second energy delivery element, an energy delivery console to provide energy between the first energy delivery element and the second energy delivery element, and a user interface having a display for providing information to a user. The energy provided by the energy delivery console creates one or more electric fields within an energy delivery volume proximate the first energy delivery element, and at least one electric field within the energy delivery volume is sufficient to irreversibly electroporate target tissue within the energy delivery volume.

### RELATED APPLICATIONS

The present application claims priority to U.S. Provisional Patent Application Ser. No. 63/226,040, entitled “ENERGY DELIVERY SYSTEMS WITH LESION INDEX”, filed Jul. 27, 2021, which is hereby incorporated by reference.

The present application claims priority to States Provisional Patent Application Ser. No. 63/336,245, entitled “ENERGY DELIVERY SYSTEMS WITH LESION INDEX”, filed Apr. 28, 2022, which is hereby incorporated by reference.

The present application, while not claiming priority to, may be related to U.S. Provisional Application Ser. No. 63/203,606, entitled “Tissue Treatment System”, filed Jul. 27, 2021, which is hereby incorporated by reference.

The present application, while not claiming priority to, may be related to U.S. Provisional Application Ser. No. 63/260,234, entitled “Intravascular Atrial Fibrillation Treatment”, filed Aug. 13, 2021, which is hereby incorporated by reference.

The present application, while not claiming priority to, may be related to US national stage filing of Patent Cooperation Treaty Application No. PCT/US2022/016722, entitled “Energy Delivery Systems With Ablation Index”, filed Feb. 17, 2022, which claims priority to U.S. Provisional Application Ser. No. 63/150,555, entitled “Energy Delivery Systems With Ablation Index”, filed Feb. 17, 2021, each of which is hereby incorporated by reference.

The present application, while not claiming priority to, may be related to U.S. application Ser. No. 16/335,893, entitled “Ablation System with Force Control”, filed Mar. 22, 2019, which is a 35 USC 371 national stage filing of Patent Cooperation Treaty Application No. PCT/US2017/056064, entitled “Ablation System with Force Control”, filed Oct. 11, 2017, which claims priority to U.S. Provisional Application Ser. No. 62/406,748, entitled “Ablation System with Force Control”, filed Oct. 11, 2016, and U.S. Provisional Application Ser. No. 62/504,139, entitled “Ablation System with Force Control”, filed May 10, 2017, each of which is hereby incorporated by reference.

The present application, while not claiming priority to, may be related to U.S. application Ser. No. 16/097,955, entitled “Cardiac Information Dynamic Display System and Method”, filed Oct. 31, 2018, which is a 35 USC 371 national stage filing of Patent Cooperation Treaty Application No. PCT/US2017/030915, entitled “Cardiac Information Dynamic Display System and Method”, filed May 3, 2017, which claims priority to U.S. Provisional Application Ser. No. 62/331,351, entitled “Cardiac Information Dynamic Display System and Method”, filed May 3, 2016, each of which is hereby incorporated by reference.

The present application, while not claiming priority to, may be related to U.S. patent application Ser. No. 16/861,814, entitled “Catheter System and Methods of Medical Uses of Same, including Diagnostic and Treatment Uses for the Heart”, filed Apr. 29, 2020, which is a continuation of U.S. Pat. No. 10,667,753, entitled “Catheter System and Methods of Medical Uses of Same, Including Diagnostic and Treatment Uses for the Heart”, filed Jun. 19, 2018, which is a continuation of U.S. Pat. No. 10,004,459, entitled “Catheter System and Methods of Medical Uses of Same, Including Diagnostic and Treatment Uses for the Heart”, filed Feb. 20, 2015, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/242,810, entitled “Expandable Catheter Assembly with Flexible Printed Circuit Board (PCB) Electrical Pathways”, filed Jan. 8, 2019, which is a continuation of U.S. Pat. No. 10,201,311, entitled “Expandable Catheter Assembly with Flexible Printed Circuit Board (PCB) Electrical Pathways”, filed Jul. 23, 2015, which is a 35 USC 371 national stage filing of Patent Cooperation Treaty Application No. PCT/US2014/015261, entitled “Expandable Catheter Assembly with Flexible Printed Circuit Board (PCB) Electrical Pathways”, filed Feb. 7, 2014, which claims priority to U.S. Patent Provisional Application Ser. No. 61/762,363, entitled “Expandable Catheter Assembly with Flexible Printed Circuit Board (PCB) Electrical Pathways”, filed Feb. 8, 2013, each of which is hereby incorporated by reference.

The present application, while not claiming priority to, may be related to U.S. patent application Ser. No. 16/533,028, entitled “Method and Device for Determining and Presenting Surface Charge and Dipole Densities on Cardiac Walls”, filed Aug. 6, 2019, which is a continuation of U.S. Pat. No. 10,413,206, entitled “Method and Device for Determining and Presenting Surface Charge and Dipole Densities on Cardiac Walls”, filed Jun. 21, 2018, which is a continuation of U.S. Pat. No. 10,376,171, entitled “Method and Device for Determining and Presenting Surface Charge and Dipole Densities on Cardiac Walls”, filed Feb. 17, 2017, which is a continuation of U.S. Pat. No. 9,610,024, entitled “Method and Device for Determining and Presenting Surface Charge and Dipole Densities on Cardiac Walls”, filed Sep. 25, 2015, which is a continuation of U.S. Pat. No. 9,167,982, entitled “Method and Device for Determining and Presenting Surface Charge and Dipole Densities on Cardiac Walls”, filed Nov. 19, 2014, which is a continuation of U.S. Pat. No. 8,918,158, entitled “Method and Device for Determining and Presenting Surface Charge and Dipole Densities on Cardiac Walls”, filed Feb. 25, 2014, which is a continuation of U.S. Pat. No. 8,700,119, entitled “Method and Device for Determining and Presenting Surface Charge and Dipole Densities on Cardiac Walls”, filed Apr. 8, 2013, which is a continuation of U.S. Pat. No. 8,417,313, entitled “Method and Device for Determining and Presenting Surface Charge and Dipole Densities on Cardiac Walls”, filed Feb. 3, 2009, which is a 35 USC 371 national stage filing of PCT Application No. PCT/CH2007/000380, entitled “Method and Device for Determining and Presenting Surface Charge and Dipole Densities on Cardiac Walls”, filed Aug. 3, 2007, which claims priority to Swiss Patent Application No. 1251/06, filed Aug. 3, 2006, each of which is hereby incorporated by reference.

The present application, while not claiming priority to, may be related to U.S. Pat. No. 11,116,438, entitled “Device and Method for the Geometric Determination of Electrical Dipole Densities on the Cardiac Wall”, filed Sep. 12, 2019, which is a continuation of U.S. Pat. No. 10,463,267, entitled “Device and Method for the Geometric Determination of Electrical Dipole Densities on the Cardiac Wall”, filed Jan. 29, 2018, which is a continuation of U.S. Pat. No. 9,913,589, entitled “Device and Method for the Geometric Determination of Electrical Dipole Densities on the Cardiac Wall”, filed Oct. 25, 2016, which is a continuation of U.S. Pat. No. 9,504,395, entitled “Device and Method for the Geometric Determination of Electrical Dipole Densities on the Cardiac Wall”, filed Oct. 19, 2015, which is a continuation of U.S. Pat. No. 9,192,318, entitled “Device and Method for the Geometric Determination of Electrical Dipole Densities on the Cardiac Wall”, filed Jul. 19, 2013, which is a continuation of U.S. Pat. No. 8,512,255, entitled “Device and Method for the Geometric Determination of Electrical Dipole Densities on the Cardiac Wall”, filed Jul. 16, 2010, which is a 35 USC 371 national stage application of Patent Cooperation Treaty Application No. PCT/IB2009/000071, filed Jan. 16, 2009, entitled “A Device and Method for the Geometric Determination of Electrical Dipole Densities on the Cardiac Wall”, which claimed priority to Swiss Patent Application 00068/08 filed Jan. 17, 2008, each of which is hereby incorporated by reference.

The present application, while not claiming priority to, may be related to U.S. patent application Ser. No. 17/673,995, entitled “Device and Method for the Geometric Determination of Electrical Dipole Densities on the Cardiac Wall”, filed Feb. 17, 2022, which is a continuation of U.S. Pat. No. 11,278,209, entitled “Device and Method for the Geometric Determination of Electrical Dipole Densities on the Cardiac Wall”, filed Apr. 19, 2019, which is a continuation of U.S. Pat. No. 10,314,497, entitled “Device and Method for the Geometric Determination of Electrical Dipole Densities on the Cardiac Wall”, filed Mar. 20, 2018, which is a continuation of U.S. Pat. No. 9,968,268, entitled “Device and Method for the Geometric Determination of Electrical Dipole Densities on the Cardiac Wall”, filed Aug. 8, 2017, which is a continuation of U.S. Pat. No. 9,757,044, entitled “Device and Method for the Geometric Determination of Electrical Dipole Densities on the Cardiac Wall”, filed Sep. 6, 2013, which is a 35 USC 371 national stage filing of Patent Cooperation Treaty Application No. PCT/US2012/028593, entitled “Device and Method for the Geometric Determination of Electrical Dipole Densities on the Cardiac Wall”, filed Mar. 9, 2012, which claimed priority to U.S. Patent Provisional Application Ser. No. 61/451,357, filed Mar. 10, 2011, each of which is hereby incorporated by reference.

The present application, while not claiming priority to, may be related to US Design Patent No. 29/681,827, entitled “Set of Transducer-Electrode Pairs for a Catheter”, filed Feb. 28, 2019, which is a division of US Design Pat. No. D851,774, entitled “Set of Transducer-Electrode Pairs for a Catheter”, filed Feb. 6, 2017, which is a division of US Design Pat. No. D782,686, entitled “Transducer-Electrode Pair for a Catheter”, filed Dec. 2, 2013, which is a 35 USC 371 national stage filing of Patent Cooperation Treaty Application No. PCT/US2013/057579, entitled “Catheter System and Methods of Medical Uses of Same, Including Diagnostic and Treatment Uses for the Heart”, filed Aug. 30, 2013, which claims priority to U.S. Patent Provisional Application Ser. No. 61/695,535, entitled “System and Method for Diagnosing and Treating Heart Tissue”, filed Aug. 31, 2012, each of which is hereby incorporated by reference.

The present application, while not claiming priority to, may be related to U.S. patent application Ser. No. 16/111,538, entitled “Gas-Elimination Patient Access Device”, filed Aug. 24, 2018, which is a continuation of U.S. Pat. No. 10,071,227, entitled “Gas-Elimination Patient Access Device”, filed Jul. 14, 2016, which is a 35 USC 371 national stage filing of Patent Cooperation Treaty Application No. PCT/US2015/011312, entitled “Gas-Elimination Patient Access Device”, filed Jan. 14, 2015, which claims priority to U.S. Patent Provisional Application Ser. No. 61/928,704, entitled “Gas-Elimination Patient Access Device”, filed Jan. 17, 2014, which is hereby incorporated by reference.

The present application, while not claiming priority to, may be related to U.S. patent application Ser. No. 17/578,522, entitled “Cardiac Analysis User Interface System and Method”, filed Jan. 19, 2022, which is a continuation of U.S. Pat. No. 11,278,231, entitled “Cardiac Analysis User Interface System and Method”, filed Sep. 23, 2016, which is a 35 USC 371 national stage filing of Patent Cooperation Treaty Application No. PCT/US2015/022187, entitled “Cardiac Analysis User Interface System and Method”, filed Mar. 24, 2015, which claims priority to U.S. Patent Provisional Application Ser. No. 61/970,027, entitled “Cardiac Analysis User Interface System and Method”, filed Mar. 25, 2014, which is hereby incorporated by reference.

The present application, while not claiming priority to, may be related to U.S. patent application Ser. No. 17/063,901, entitled “Devices and Methods for Determination of Electrical Dipole Densities on a Cardiac Surface”, filed Oct. 6, 2020, which is a continuation of U.S. Pat. No. 10,828,011, entitled “Devices and Methods for Determination of Electrical Dipole Densities on a Cardiac Surface”, filed Mar. 2, 2016, which is a 35 USC 371 national stage filing of Patent Cooperation Treaty Application No. PCT/US2014/054942, entitled “Devices and Methods for Determination of Electrical Dipole Densities on a Cardiac Surface”, filed Sep. 10, 2014, which claims priority to U.S. Patent Provisional Application Ser. No. 61/877,617, entitled “Devices and Methods for Determination of Electrical Dipole Densities on a Cardiac Surface”, filed Sep. 13, 2013, which is hereby incorporated by reference.

The present application, while not claiming priority to, may be related to U.S. patent application Ser. No. 16/849,045, entitled “Localization System and Method Useful in the Acquisition and Analysis of Cardiac Information”, filed Apr. 15, 2020, which is a continuation of U.S. Pat. No. 10,653,318, entitled “Localization System and Method Useful in the Acquisition and Analysis of Cardiac Information”, filed Oct. 26, 2017, which is a 35 USC 371 national stage filing of Patent Cooperation Treaty Application No. PCT/US2016/032420, entitled “Localization System and Method Useful in the Acquisition and Analysis of Cardiac Information”, filed May 13, 2016, which claims priority to U.S. Patent Provisional Application Ser. No. 62/161,213, entitled “Localization System and Method Useful in the Acquisition and Analysis of Cardiac Information”, filed May 13, 2015, which is hereby incorporated by reference.

The present application, while not claiming priority to, may be related to U.S. patent application Ser. No. 15/569,231, entitled “Cardiac Virtualization Test Tank and Testing System and Method”, filed Oct. 25, 2017, which is a 35 USC 371 national stage filing of Patent Cooperation Treaty Application No. PCT/US2016/031823, filed May 11, 2016, which claims priority to U.S. Patent Provisional Application Ser. No. 62/160,501, entitled “Cardiac Virtualization Test Tank and Testing System and Method”, filed May 12, 2015, which is hereby incorporated by reference.

The present application, while not claiming priority to, may be related to U.S. patent application Ser. No. 17/735,285, entitled “Ultrasound Sequencing System and Method”, filed May 3, 2022, which is a continuation of to U.S. patent application Ser. No. 15/569,185, entitled “Ultrasound Sequencing System and Method”, filed Oct. 25, 2017, which is a 35 USC 371 national stage filing of Patent Cooperation Treaty Application No. PCT/US2016/032017, filed May 12, 2016, which claims priority to U.S. Patent Provisional Application Ser. No. 62/160,529, entitled “Ultrasound Sequencing System and Method”, filed May 12, 2015, which is hereby incorporated by reference.

The present application, while not claiming priority to, may be related to U.S. patent application Ser. No. 17/858,174, entitled “Cardiac Mapping System with Efficiency Algorithm”, filed Jul. 6, 2022, which is a Continuation application of U.S. patent application Ser. No. 16/097,959, entitled “Cardiac Mapping System with Efficiency Algorithm”, filed Oct. 31, 2018, which is a 35 USC 371 national stage filing of Patent Cooperation Treaty Application No. PCT/US2017/030922, entitled “Cardiac Mapping System with Efficiency Algorithm”, filed May 3, 2017, which claims priority to U.S. Patent Provisional Application Ser. No. 62/413,104, entitled “Cardiac Mapping System with Efficiency Algorithm”, filed Oct. 26, 2016, and U.S. Patent Provisional Application Ser. No. 62/331,364, entitled “Cardiac Mapping System with Efficiency Algorithm”, filed May 3, 2016, each of which is hereby incorporated by reference.

The present application, while not claiming priority to, may be related to U.S. patent application Ser. No. 16/961,809, entitled “System for Identifying Cardiac Conduction Patterns”, filed Jul. 13, 2020, which is a 35 USC 371 national stage filing of Patent Cooperation Treaty Application No. PCT/US2019/014498, entitled “System for Identifying Cardiac Conduction Patterns”, filed Jan. 22, 2019, which claims priority to U.S. Patent Provisional Application Ser. No. 62/619,897, entitled “System for Recognizing Cardiac Conduction Patterns”, filed Jan. 21, 2018, and U.S. Patent Provisional Application Ser. No. 62/668,647, entitled “System for Identifying Cardiac Conduction Patterns”, filed May 8, 2018, each of which is hereby incorporated by reference.

The present application, while not claiming priority to, may be related to U.S. patent application Ser. No. 17/048,151, entitled “Cardiac Information Processing System”, filed Oct. 16, 2020, which is a 35 USC 371 national stage filing of Patent Cooperation Treaty Application No. PCT/US2019/031131, entitled “Cardiac Information Processing System”, filed May 7, 2019, which claims priority to U.S. Provisional Application Ser. No. 62/668,659, entitled “Cardiac Information Processing System”, filed May 8, 2018, and U.S. Patent Provisional Application Ser. No. 62/811,735, entitled “Cardiac Information Processing System”, filed Feb. 28, 2019, each of which is hereby incorporated by reference.

The present application, while not claiming priority to, may be related to Patent Cooperation Treaty Application No. PCT/US2019/060433, entitled “Systems and Methods for Calculating Patient Information”, filed Nov. 8, 2019, which claims priority to U.S. Provisional Application Ser. No. 62/757,961, entitled “Systems and Methods for Calculating Patient Information”, filed Nov. 9, 2018, each of which is hereby incorporated by reference.

The present application, while not claiming priority to, may be related to U.S. patent application Ser. No. 17/601,661, entitled “System for Creating a Composite Map”, filed Oct. 5, 2021, which is a 35 USC 371 national stage filing of Patent Cooperation Treaty Application No. PCT/US2020/028779, entitled “System for Creating a Composite Map”, filed Apr. 17, 2020, which claims priority to U.S. Provisional Application Ser. No. 62/835,538, entitled “System for Creating a Composite Map”, filed Apr. 18, 2019, and U.S. Provisional Application Ser. No. 62/925,030, entitled “System for Creating a Composite Map”, filed Oct. 23, 2019, each of which is hereby incorporated by reference.

The present application, while not claiming priority to, may be related to U.S. patent application Ser. No. 17/613,249, entitled “Systems And Methods For Performing Localization Within A Body”, filed Nov. 22, 2021, which is a 35 USC 371 national stage filing of Patent Cooperation Treaty Application No. PCT/US2020/036110, entitled “Systems and Methods for Performing Localization Within a Body”, filed Jun. 4, 2020, which claims priority to U.S. Provisional Application Ser. No. 62/857,055, entitled “Systems and Methods for Performing Localization Within a Body”, filed Jun. 4, 2019, each of which is hereby incorporated by reference.

The present application, while not claiming priority to, may be related to U.S. patent application Ser. No. 17/777,104, entitled “Tissue Treatment Systems, Devices, and Methods”, filed May 16, 2022, which is a 35 USC 371 national stage filing of Patent Cooperation Treaty Application No. PCT/US2020/061458, entitled “Tissue Treatment Systems, Devices, and Methods”, filed Nov. 20, 2020, which claims priority to U.S. Provisional Application Ser. No. 62/939,412, entitled “Tissue Treatment Systems, Devices, and Methods”, filed Nov. 22, 2019, and U.S. Provisional Application Ser. No. 63/075,280, entitled “Tissue Treatment Systems, Devices, and Methods”, filed Sep. 7, 2020, each of which is hereby incorporated by reference.

### FIELD OF THE PRESENT INVENTIVE CONCEPTS

The present inventive concepts relate generally to systems, devices, and methods for ablating tissue, and in particular, for ablating tissue of a patient's heart.

### BACKGROUND

Numerous medical procedures include the delivery of energy to ablate or otherwise treat tissue. Achieving desired specificity and efficacy of tissue treatment can be challenging, and the inability to do so can result in less than desired results.

There is a need for systems, methods, and devices that achieve improved tissue treatment via delivery of energy.

### SUMMARY

According to an aspect of the present inventive concepts, a system for treating tissue of a patient comprises: a first energy delivery device comprising a first energy delivery element configured to be positioned proximate target tissue of the patient; a second energy delivery element; an energy delivery console configured to provide energy between the first energy delivery element and the second energy delivery element; and a user interface including a display for providing information to a user. The energy provided by the energy delivery console creates one or more electric fields within an energy delivery volume proximate the first energy delivery element, and at least one electric field within the energy delivery volume is sufficient to irreversibly electroporate target tissue within the energy delivery volume.

In some embodiments, one of the first and/or the second energy delivery element is configured to source current, and the other of the second and/or the first energy delivery element is configured to sink current.

In some embodiments, the second energy delivery element is positioned on the first energy delivery device.

In some embodiments, the system further comprises a second energy delivery device, and the second energy delivery element is positioned on the second energy delivery device. The second energy delivery device can comprise a flexible filament constructed and arranged similar to a guidewire. The second energy delivery device can be constructed and arranged to be positioned within an epicardial vessel. The first energy delivery element can be configured to be positioned proximate the target tissue from within a cardiac chamber, and the second energy delivery element can be configured to be positioned proximate the target tissue from within an epicardial vessel.

In some embodiments, the first energy delivery device comprises a set of multiple energy delivery elements, and the first energy delivery element and the second energy delivery element are selected by a user from the set of multiple energy delivery elements. The first and second energy delivery elements can be selected based on the desired shape and/or size of the energy delivery volume.

In some embodiments, the system comprises a set of adjustable parameters that determine the configuration of the provided energy. The adjustable parameters can be selected from the group consisting of: voltage; current; frequency; pulse width; and combinations thereof.

In some embodiments, the system further comprises a controller, memory coupled to the controller, and an algorithm, and the memory is configured to store instructions for the controller to perform the algorithm. The algorithm can be configured to determine a lesion parameter of a to be created lesion and/or an already created lesion. The lesion parameter can be selected from the group consisting of: a length; a width; a depth; a volume; and combinations thereof. The lesion parameter can be based on: the peak voltage of the provided energy; an orientation of the first and second energy delivery elements relative to the target tissue; and/or one or more electrophysical parameters of the patient and/or the system. The one or more electrophysical parameters can be selected from the group consisting of: contact force; pulse amplitude; pulse duration; number of pulses; number of energy delivery elements delivering the energy; tissue temperature; tissue impedance; and combinations thereof. The system can be configured to determine the orientation of the first and second energy delivery elements relative to the target tissue via localization of the energy delivery elements. The localization can comprise magnetic localization and/or impedance localization.

In some embodiments, the system is configured to provide a graphical user interface (GUI) on the user interface. The GUI can include an overlay representing the size, shape, and/or position of the energy delivery volume. The overlay can be displayed to the user prior to the creation of the one or more electric fields. The appearance of the overlay can be based on the configuration of the provided energy. The overlay can comprise multiple layers, and each layer can indicate a variation in the energy delivery volume. The overlay can comprise one or more visual properties, and the one or more visual properties can vary based on the properties of the energy delivery volume. The one or more visual properties can vary based on a comparison of the energy delivery volume to a threshold. The threshold can be a threshold depth into myocardial tissue. The GUI can include an overlay representing the probability that tissue within the energy delivery volume will be efficaciously treated by the creation of one or more electric fields. The GUI can include an overlay representing the probability that target tissue will be efficaciously treated by the creation of one or more electric fields. The GUI can display information related to the angle of orientation between the first energy delivery device and the target tissue. The GUI can include a tissue representation comprising a computer-generated anatomic model of at least a portion of the patient's heart. The tissue representation can comprise a shell representing the interior surface of a heart chamber. The shell can comprise zero thickness. At least a portion of the shell can comprise a thickness representing the thickness of the myocardium. The GUI can include an overlay representing the energy delivery volume as displayed relative to the shell. A portion of the energy delivery volume positioned outside of the shell can be represented by the overlay.

The technology described herein, along with the attributes and attendant advantages thereof, will best be appreciated and understood in view of the following detailed description taken in conjunction with the accompanying drawings in which representative embodiments are described by way of example.

According to an aspect of the present inventive concepts, a method for treating tissue of a patient comprises: providing a first energy delivery device comprising a first energy delivery element configured to be positioned proximate target tissue of the patient; providing a second energy delivery element; navigating the first energy delivery device to the target tissue; an energy delivery console providing energy between the first energy delivery element and the second energy delivery element; and generating a display on a user interface device comprising tissue and/or energy delivery volume information. The energy provided by the energy delivery console creates one or more electric fields within an energy delivery volume proximate the first energy delivery element, and wherein at least one electric field within the energy delivery volume is sufficient to irreversibly electroporate target tissue within the energy delivery volume.

In some embodiments, the method includes one of the first and/or the second energy delivery element sourcing current, and the other of the second and/or the first energy delivery element sinking current.

In some embodiments, the second energy delivery element is positioned on the first energy delivery device.

In some embodiments, the method includes providing a second energy delivery device, wherein the second energy delivery element is positioned on the second energy delivery device.

In some embodiments, the second energy delivery device comprises a flexible filament constructed and arranged similar to a guidewire.

In some embodiments, the second energy delivery device is constructed and arranged to be positioned within an epicardial vessel.

In some embodiments, the method includes positioning the first energy delivery element proximate the target tissue from within a cardiac chamber, and positioning the second energy delivery element proximate the target tissue from within an epicardial vessel.

In some embodiments, the first energy delivery device comprises a set of multiple energy delivery elements, and wherein the first energy delivery element and the second energy delivery element are selected by a user from the set of multiple energy delivery elements.

In some embodiments, the first and second energy delivery elements are selected based on the desired shape and/or size of the energy delivery volume.

In some embodiments, the method includes providing the energy delivery console with a set of adjustable parameters that determine the configuration of the provided energy.

In some embodiments, the adjustable parameters are selected from the group consisting of: voltage; current; frequency; pulse width; and combinations thereof.

In some embodiments, the energy delivery console comprises a controller, a memory coupled to the controller, and an algorithm, wherein the memory is configured to store instructions for the controller to perform the algorithm.

In some embodiments, the method includes performing the algorithm to determine a lesion parameter of a to be created lesion and/or an already created lesion.

In some embodiments, the lesion parameter is selected from the group consisting of: a length; a width; a depth; a volume; and combinations thereof.

In some embodiments, the lesion parameter is based on: the peak voltage of the provided energy; an orientation of the first and second energy delivery elements relative to the target tissue; and/or one or more electrophysical parameters of the patient and/or the system.

In some embodiments, the one or more electrophysical parameters are selected from the group consisting of: contact force; pulse amplitude; pulse duration; number of pulses; number of energy delivery elements delivering the energy; tissue temperature; tissue impedance; and combinations thereof.

In some embodiments, the method includes determining the orientation of the first and second energy delivery elements relative to the target tissue via localization of the energy delivery elements.

In some embodiments, the localization comprises magnetic localization and/or impedance localization.

In some embodiments, the method includes generating a graphical user interface (GUI) on the user interface.

In some embodiments, generating the GUI includes displaying an overlay representing the size, shape, and/or position of the energy delivery volume.

In some embodiments, the method includes displaying the overlay via the GUI prior to the creation of the one or more electric fields.

In some embodiments, the appearance of the overlay is based on the configuration of the provided energy.

In some embodiments, the overlay comprises multiple layers, and wherein each layer indicates a variation in the energy delivery volume.

In some embodiments, the overlay comprises one or more visual properties, and wherein the one or more visual properties vary based on the properties of the energy delivery volume.

In some embodiments, the method includes varying the one or more visual properties based on a comparison of the energy delivery volume to a threshold.

In some embodiments, the threshold is a threshold depth into myocardial tissue.

In some embodiments, the method includes displaying the overlay via the GUI representing the probability that tissue within the energy delivery volume will be efficaciously treated by the creation of one or more electric fields.

In some embodiments, the method includes displaying the overlay via the GUI representing the probability that target tissue will be efficaciously treated by the creation of one or more electric fields.

In some embodiments, the method includes displaying information via the GUI related to the angle of orientation between the first energy delivery device and the target tissue.

In some embodiments, the method includes displaying a tissue representation via the GUI comprising a computer-generated anatomic model of at least a portion of the patient's heart.

In some embodiments, the tissue representation comprises a shell representing the interior surface of a heart chamber.

In some embodiments, the shell comprises zero thickness.

In some embodiments, at least a portion of the shell comprises a thickness representing the thickness of the myocardium.

In some embodiments, the method includes displaying an overlay via the GUI representing the energy delivery volume as displayed relative to the shell.

In some embodiments, only the portion of the energy delivery volume positioned outside of the shell is represented by the overlay.

### INCORPORATION BY REFERENCE

All publications, patents, and patent applications mentioned in this specification are herein incorporated by reference to the same extent as if each individual publication, patent, or patent application was specifically and individually indicated to be incorporated by reference. The content of all publications, patents, and patent applications mentioned in this specification are herein incorporated by reference in their entirety for all purposes.

### BRIEF DESCRIPTION OF THE DRAWINGS

FIG. 1 illustrates a schematic view of an embodiment of a system for performing a medical procedure on a patient, consistent with the present inventive concepts.

FIG. 2 illustrates a flow chart of an embodiment of a method for delivering energy to tissue of a patient, consistent with the present inventive concepts.

FIG. 3 illustrates a flow chart of another embodiment of a method for delivering energy to tissue of a patient, consistent with the present inventive concepts.

FIG. 3A illustrates a graph of a heart cycle including a desired cycle point, consistent with the present inventive concepts.

FIG. 4 illustrates a flow chart of another embodiment of a method for delivering energy to tissue of a patient, consistent with the present inventive concepts.

FIG. 5 illustrates a flow chart of another embodiment of a method for delivering energy to tissue of a patient, consistent with the present inventive concepts.

FIG. 6 illustrates a flow chart of another embodiment of a method for delivering energy to tissue of a patient, consistent with the present inventive concepts.

FIG. 7 illustrates a flow chart of another embodiment of a method for delivering energy to tissue of a patient, consistent with the present inventive concepts.

FIG. 8 illustrates a side view of an embodiment of an energy delivery device, consistent with the present inventive concepts.

FIGS. 8A-B are two graphs illustrating lesion depths created with various energy delivery geometries, consistent with the present inventive concepts.

FIGS. 9A-D are four graphs illustrating lesion volumes created with various energy delivery geometries, consistent with the present inventive concepts.

FIGS. 10A-B are two anatomical sectional views of the distal portion of an embodiment of an energy delivery device contacting a tissue surface at different angles of orientation, consistent with the present inventive concepts.

FIGS. 11A-B are two user's views of an embodiment of a graphical user interface displaying information related to different angles of orientation of an energy delivery device, consistent with the present inventive concepts.

FIG. 12 is a perspective view of the distal portion of an embodiment of an energy delivery device including multiple ports for delivering irrigation fluid, consistent with the present inventive concepts.

FIG. 13 is a side sectional anatomical view of the distal portion of an embodiment of an energy delivery device in contact with a tissue surface and delivering irrigation fluid, consistent with the present inventive concepts.

FIGS. 14A-B and FIGS. 15A-C are side views of an embodiment of an energy delivery device proximate sectional portions of tissue, consistent with the present inventive concepts.

FIG. 16 is a user's view of a portion of an embodiment of a graphical user interface of a system for performing a medical procedure, consistent with the present inventive concepts.

FIG. 17 illustrates a perspective view of embodiments of two energy delivery devices positioned with a vessel and proximate a sectional portion of tissue, consistent with the present inventive concepts.

FIGS. 18A-18B illustrate a user's view of a portion of an embodiment of a graphical user interface displaying an anatomic model, consistent with the present inventive concepts.

FIG. 19 illustrates a portion of an embodiment of a graphical user interface displaying an overlay, consistent with the present inventive concepts

FIG. 20 illustrates a portion of an embodiment of a graphical user interface displaying predicted and actual treatment data, consistent with the present inventive concepts.

FIG. 21 illustrates a schematic view of an embodiment of various treatment steps, consistent with the present inventive concepts.

FIGS. 22A-B and 23A-B illustrate finite element models of various embodiments of electrode configurations, consistent with the present inventive concepts.

FIGS. 24A and 24B illustrate a user's view of an embodiment of a portion of a graphical user interface displaying an anatomic model, consistent with the present inventive concepts.

FIGS. 25A-D illustrate a user's view of an embodiment of a portion of a graphical user interface displaying data relative to a treatment threshold, consistent with the present inventive concepts.

FIG. 26 illustrates a flow chart showing a general workflow of algorithms for performing field tagging, consistent with the present inventive concepts.

FIG. 27 illustrates a graphical representation of domain delineation, consistent with the present inventive concepts.

FIGS. 28A and 28B illustrate a graph of a measured catheter parameter and various representations of the catheter, consistent with the present inventive concepts.

FIGS. 29A-C illustrate various techniques of field estimation, consistent with the present inventive concepts.

FIGS. 30A-C illustrate a representation of how a multi-level attrition profile can allow for dynamic updates to applied therapy, consistent with the present inventive concepts.

FIGS. 31A-C illustrate examples of a depth projection feature, consistent with the present inventive concepts.

FIGS. 32A-C illustrate an example of how prospective lesion set planning can be performed, consistent with the present inventive concepts.

FIGS. 33A-D illustrate an example of thickness-based therapy planning, consistent with the present inventive concepts.

FIG. 34 illustrates a UAC-based system implemented across several left atrium geometries, consistent with the present inventive concepts.

### DETAILED DESCRIPTION OF THE DRAWINGS

Reference will now be made in detail to the present embodiments of the technology, examples of which are illustrated in the accompanying drawings. Similar reference numbers may be used to refer to similar components. However, the description is not intended to limit the present disclosure to particular embodiments, and it should be construed as including various modifications, equivalents, and/or alternatives of the embodiments described herein.

It will be understood that the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) when used herein, specify the presence of stated features, integers, steps, operations, elements, and/or components, but do not preclude the presence or addition of one or more other features, integers, steps, operations, elements, components, and/or groups thereof.

It will be further understood that, although the terms first, second, third, etc. may be used herein to describe various limitations, elements, components, regions, layers and/or sections, these limitations, elements, components, regions, layers and/or sections should not be limited by these terms. These terms are only used to distinguish one limitation, element, component, region, layer or section from another limitation, element, component, region, layer or section. Thus, a first limitation, element, component, region, layer or section discussed below could be termed a second limitation, element, component, region, layer or section without departing from the teachings of the present application.

It will be further understood that when an element is referred to as being “on”, “attached”, “connected” or “coupled” to another element, it can be directly on or above, or connected or coupled to, the other element, or one or more intervening elements can be present. In contrast, when an element is referred to as being “directly on”, “directly attached”, “directly connected” or “directly coupled” to another element, there are no intervening elements present. Other words used to describe the relationship between elements should be interpreted in a like fashion (e.g. “between” versus “directly between,” “adjacent” versus “directly adjacent,” etc.).

It will be further understood that when a first element is referred to as being “in”, “on” and/or “within” a second element, the first element can be positioned: within an internal space of the second element, within a portion of the second element (e.g. within a wall of the second element); positioned on an external and/or internal surface of the second element; and combinations of one or more of these.

As used herein, the term “proximate”, when used to describe proximity of a first component or location to a second component or location, is to be taken to include one or more locations near to the second component or location, as well as locations in, on and/or within the second component or location. For example, a component positioned proximate an anatomical site (e.g. a target tissue location), shall include components positioned near to the anatomical site, as well as components positioned in, on and/or within the anatomical site.

Spatially relative terms, such as “beneath,” “below,” “lower,” “above,” “upper” and the like may be used to describe an element and/or feature's relationship to another element(s) and/or feature(s) as, for example, illustrated in the figures. It will be further understood that the spatially relative terms are intended to encompass different orientations of the device in use and/or operation in addition to the orientation depicted in the figures. For example, if the device in a figure is turned over, elements described as “below” and/or “beneath” other elements or features would then be oriented “above” the other elements or features. The device can be otherwise oriented (e.g. rotated 90 degrees or at other orientations) and the spatially relative descriptors used herein interpreted accordingly.

The terms “reduce”, “reducing”, “reduction” and the like, where used herein, are to include a reduction in a quantity, including a reduction to zero. Reducing the likelihood of an occurrence shall include prevention of the occurrence. Correspondingly, the terms “prevent”, “preventing”, and “prevention” shall include the acts of “reduce”, “reducing”, and “reduction”, respectively.

The term “and/or” where used herein is to be taken as specific disclosure of each of the two specified features or components with or without the other. For example, “A and/or B” is to be taken as specific disclosure of each of (i) A, (ii) B and (iii) A and B, just as if each is set out individually herein.

The term “one or more”, where used herein can mean one, two, three, four, five, six, seven, eight, nine, ten, or more, up to any number.

The terms “and combinations thereof” and “and combinations of these” can each be used herein after a list of items that are to be included singly or collectively. For example, a component, process, and/or other item selected from the group consisting of: A; B; C; and combinations thereof, shall include a set of one or more components that comprise: one, two, three or more of item A; one, two, three or more of item B; and/or one, two, three, or more of item C.

In this specification, unless explicitly stated otherwise, “and” can mean “or”, and “or” can mean “and”. For example, if a feature is described as having A, B, or C, the feature can have A, B, and C, or any combination of A, B, and C. Similarly, if a feature is described as having A, B, and C, the feature can have only one or two of A, B, or C.

As used herein, when a quantifiable parameter is described as having a value “between” a first value X and a second value Y, it shall include the parameter having a value of: at least X, no more than Y, and/or at least X and no more than Y. For example, a length of between 1 and 10 shall include a length of at least 1 (including values greater than 10), a length of less than 10 (including values less than 1), and/or values greater than 1 and less than 10.

The expression “configured (or set) to” used in the present disclosure may be used interchangeably with, for example, the expressions “suitable for”, “having the capacity to”, “designed to”, “adapted to”, “made to” and “capable of” according to a situation. The expression “configured (or set) to” does not mean only “specifically designed to” in hardware. Alternatively, in some situations, the expression “a device configured to” may mean that the device “can” operate together with another device or component.

As used herein, the term “threshold” refers to a maximum level, a minimum level, and/or range of values correlating to a desired or undesired state. In some embodiments, a system parameter is maintained above a minimum threshold, below a maximum threshold, within a threshold range of values, and/or outside a threshold range of values, such as to cause a desired effect (e.g. efficacious therapy) and/or to prevent or otherwise reduce (hereinafter “prevent”) an undesired event (e.g. a device and/or clinical adverse event). In some embodiments, a system parameter is maintained above a first threshold (e.g. above a first temperature threshold to cause a desired therapeutic effect to tissue) and below a second threshold (e.g. below a second temperature threshold to prevent undesired tissue damage). In some embodiments, a threshold value is determined to include a safety margin, such as to account for patient variability, system variability, tolerances, and the like. As used herein, “exceeding a threshold” relates to a parameter going above a maximum threshold, below a minimum threshold, within a range of threshold values and/or outside of a range of threshold values.

As described herein, “room pressure” shall mean pressure of the environment surrounding the systems and devices of the present inventive concepts. Positive pressure includes pressure above room pressure or simply a pressure that is greater than another pressure, such as a positive differential pressure across a fluid pathway component such as a valve. Negative pressure includes pressure below room pressure or a pressure that is less than another pressure, such as a negative differential pressure across a fluid component pathway such as a valve. Negative pressure can include a vacuum but does not imply a pressure below a vacuum. As used herein, the term “vacuum” can be used to refer to a full or partial vacuum, or any negative pressure as described hereabove.

The term “diameter” when used herein to describe a non-circular geometry is to be taken as the diameter of a hypothetical circle approximating the geometry being described. For example, when describing a cross section, such as the cross section of a component, the term “diameter” shall be taken to represent the diameter of a hypothetical circle with the same cross-sectional area as the cross section of the component being described.

The terms “major axis” and “minor axis” of a component when used herein are the length and diameter, respectively, of the smallest volume hypothetical cylinder which can completely surround the component.

As used herein, the term “functional element” is to be taken to include one or more elements constructed and arranged to perform a function. A functional element can comprise a sensor and/or a transducer. In some embodiments, a functional element is configured to deliver energy and/or otherwise treat tissue (e.g. a functional element configured as a treatment element). Alternatively or additionally, a functional element (e.g. a functional element comprising a sensor) can be configured to record one or more parameters, such as a patient physiologic parameter; a patient anatomical parameter (e.g. a tissue geometry parameter); a patient environment parameter; and/or a system parameter. In some embodiments, a sensor or other functional element is configured to perform a diagnostic function (e.g. to gather data used to perform a diagnosis). In some embodiments, a functional element is configured to perform a therapeutic function (e.g. to deliver therapeutic energy and/or a therapeutic agent). In some embodiments, a functional element comprises one or more elements constructed and arranged to perform a function selected from the group consisting of: deliver energy; extract energy (e.g. to cool a component); deliver a drug or other agent; manipulate a system component or patient tissue; record or otherwise sense a parameter such as a patient physiologic parameter or a system parameter; and combinations of one or more of these. A functional element can comprise a fluid and/or a fluid delivery system. A functional element can comprise a reservoir, such as an expandable balloon or other fluid-maintaining reservoir. A “functional assembly” can comprise an assembly constructed and arranged to perform a function, such as a diagnostic and/or therapeutic function. A functional assembly can comprise an expandable assembly. A functional assembly can comprise one or more functional elements.

The term “transducer” when used herein is to be taken to include any component or combination of components that receives energy or any input, and produces an output. For example, a transducer can include an electrode that receives electrical energy, and distributes the electrical energy to tissue (e.g. based on the size of the electrode). In some configurations, a transducer converts an electrical signal into any output, such as: light (e.g. a transducer comprising a light emitting diode or light bulb), sound (e.g. a transducer comprising a piezo crystal configured to deliver ultrasound energy); pressure (e.g. an applied pressure or force); heat energy; cryogenic energy; chemical energy; mechanical energy (e.g. a transducer comprising a motor or a solenoid); magnetic energy; and/or a different electrical signal (e.g. different than the input signal to the transducer). Alternatively or additionally, a transducer can convert a physical quantity (e.g. variations in a physical quantity) into an electrical signal. A transducer can include any component that delivers energy and/or an agent to tissue, such as a transducer configured to deliver one or more of: electrical energy to tissue (e.g. a transducer comprising one or more electrodes); light energy to tissue (e.g. a transducer comprising a laser, light emitting diode and/or optical component such as a lens or prism); mechanical energy to tissue (e.g. a transducer comprising a tissue manipulating element); sound energy to tissue (e.g. a transducer comprising a piezo crystal); chemical energy; electromagnetic energy; magnetic energy; and combinations of one or more of these.

As used herein, the term “fluid” can refer to a liquid, gas, gel, or any flowable material, such as a material which can be propelled through a lumen and/or opening.

As used herein, the term “material” can refer to a single material, or a combination of two, three, four, or more materials.

It is appreciated that certain features of the inventive concepts, which are, for clarity, described in the context of separate embodiments, may also be provided in combination in a single embodiment. Conversely, various features of the inventive concepts which are, for brevity, described in the context of a single embodiment, may also be provided separately or in any suitable sub-combination. For example, it will be appreciated that all features set out in any of the claims (whether independent or dependent) can be combined in any given way.

It is to be understood that at least some of the figures and descriptions of the inventive concepts have been simplified to focus on elements that are relevant for a clear understanding of the inventive concepts, while eliminating, for purposes of clarity, other elements that those of ordinary skill in the art will appreciate may also comprise a portion of the inventive concepts. However, because such elements are well known in the art, and because they do not necessarily facilitate a better understanding of the inventive concepts, a description of such elements is not provided herein.

Terms defined in the present disclosure are only used for describing specific embodiments of the present disclosure and are not intended to limit the scope of the present disclosure. Terms provided in singular forms are intended to include plural forms as well, unless the context clearly indicates otherwise. All of the terms used herein, including technical or scientific terms, have the same meanings as those generally understood by an ordinary person skilled in the related art, unless otherwise defined herein. Terms defined in a generally used dictionary should be interpreted as having meanings that are the same as or similar to the contextual meanings of the relevant technology and should not be interpreted as having ideal or exaggerated meanings, unless expressly so defined herein. In some cases, terms defined in the present disclosure should not be interpreted to exclude the embodiments of the present disclosure.

Provided herein are systems, devices, and methods for treating target tissue of a patient, such as to provide a therapeutic benefit to the patient. An energy delivery console can be configured to deliver various “doses” of energy to be delivered by one or more energy delivery devices to ablate, cause the necrosis of, and/or otherwise therapeutically modify the target tissue. The one or more energy delivery devices can include catheters and/or surgical tools that include electrodes and/or other energy delivery elements. The system can include a user interface including a display that is configured to provide tissue and/or energy delivery volume information to a user. In some embodiments, multiple, inter-dependent energy doses are delivered to a common tissue location, such as to provide an improved therapeutic benefit to the patient. An initial dose can be configured to warm tissue, such as a delivery of radiofrequency (RF), heat, and/or other energy. A subsequent dose can comprise a dose of energy configured to irreversibly electroporate the tissue that had previously been warmed, such as while that tissue is in an elevated temperature (e.g. above body temperature) state.

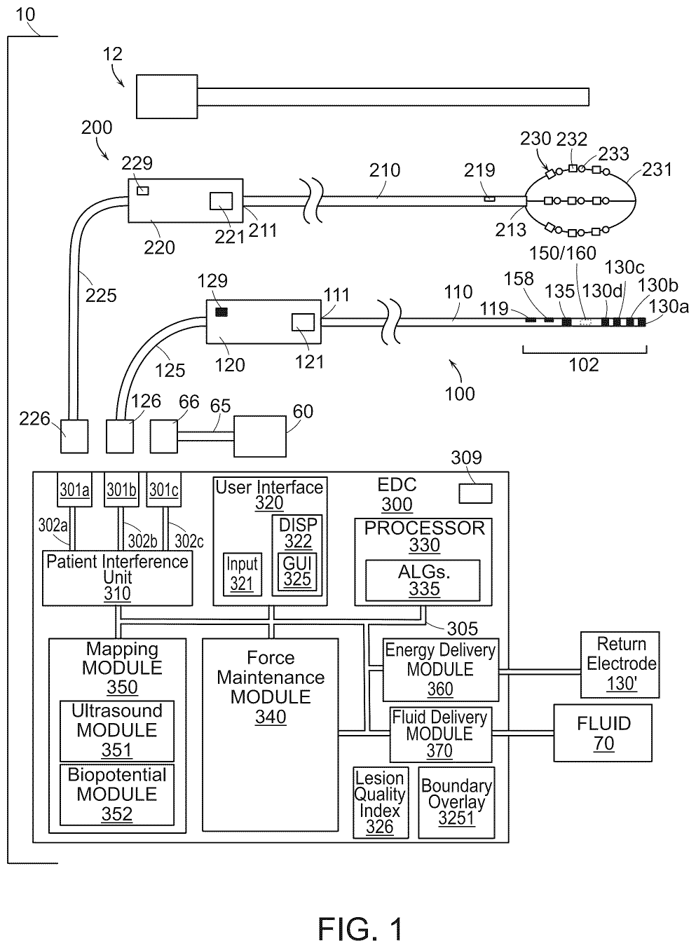

Referring now to FIG. 1, a schematic view of an embodiment of a system for performing a medical procedure on a patient (e.g. a human or other living mammal) is illustrated, consistent with the present inventive concepts. The medical procedure can comprise a diagnostic procedure, a therapeutic procedure, or a combined diagnostic and therapeutic procedure. System 10 can comprise one or more ablation catheters and/or other energy delivery devices, EDD 100, one or more mapping devices, mapping catheter 200, one or more sheaths, sheath 12, one or more patient electrode patches, patch 60, and/or a console for delivering energy, EDC 300. EDC 300 operably attaches (e.g. electrically, mechanically, fluidly, sonically, and/or optically attaches) to the one or more devices 100, 200 (e.g., two, three or more devices 100, 200), and/or to the one or more patches 60. System 10 can be configured to provide tissue (e.g. target tissue) and/or energy delivery information to a user, such as information related to a previous energy delivery performed by system 10, and/or information related to a future energy delivery to be performed by system 10.

EDC 300 can comprise a console or other device configured to deliver one or more forms of energy (e.g. deliver energy to tissue via a catheter or other energy delivery device of system 10). As used herein, delivery of energy to tissue shall include the transfer of energy to tissue (e.g. to heat, ablate, and/or otherwise affect target tissue) as well as the extraction of energy from tissue (e.g. to cool, freeze, and/or cryogenically ablate target tissue). Alternatively or additionally, delivery of energy to tissue as used herein shall include the creation of an electric field, such as an electric field of sufficient strength to modify the cellular structure of target tissue. For example, the delivery of energy to tissue can comprise the delivery of one or more pulsed field ablation pulses (e.g. between two or more electrodes), where the one or more pulses generate electric fields configured to “open” the cellular structure of the target tissue (e.g. to irreversibly electroporate target tissue). EDC 300 can be configured to deliver energy to tissue (e.g. via EDD 100) to create a lesion in target tissue, such as to create one or more therapeutic lesions in heart tissue (also referred to as “cardiac tissue” herein), such as to treat atrial fibrillation (AF), and/or other arrhythmia of the patient.

EDC 300 can deliver the one or more forms of energy to one or more electrodes and/or other energy delivery elements 130 of EDD 100 (also referred to as electrodes 130 herein). In FIG. 1, EDD 100 comprises four energy delivery elements 130, an element at the distal end of EDD 100, element 130a (e.g. a “tip electrode”), and three elements 130 mounted more proximally, elements 130b-d (e.g. “ring electrodes”), each as shown. In some embodiments, EDD 100 comprises between 1 and 64 energy delivery elements 130, such as between 1 and 12 elements 130 positioned in a linear or curvilinear arrangement. In some embodiments, EDD 100 comprises a device configured to deliver energy from inside the vasculature (e.g. from within a vein or artery). For example, EDD 100 can comprise a device constructed and arranged to be advanced through the vasculature of the heart, such as to be positioned within an epicardial vessel proximate a heart chamber. EDD 100 can comprise a flexible filament constructed and arranged similar to a guidewire (e.g. comprising similar dimensions and/or flexibility) and EDD 100 can include at least one energy delivery element 130. In some embodiments, EDC 300 is configured to deliver energy to and/or between any two electrodes of system 10. For example, EDC 300 can deliver energy between an energy delivery element 130 of EDD 100 and an electrode of a second device, such as a second EDD 100 (e.g. an EDD 100 configured as a guidewire), and/or mapping catheter 200. In some embodiments, EDD 100 is constructed and arranged to be positioned within the pericardial space (such as to deliver energy to one or more portions of the epicardium).

In some embodiments, EDC 300 can be configured to deliver a first dose of energy, dose DOE1, and a second dose of energy, dose DOE2, where dose DOE2 is different than dose DOE1 (e.g. doses DOE1 and DOE2 comprise different types of energy, levels of energy, waveforms of energy delivery, durations of energy delivery, and/or other differing energy parameter). In some embodiments, doses DOE1 and DOE2 have multiple differences in energy delivery parameters between them. In some embodiments, dose DOE1 is delivered to a first portion of tissue, and dose DOE2 is delivered to a second portion of tissue. The first and second portions of tissue can be the same portion of tissue (e.g. when sequential deliveries of energy are made to the same tissue location). In some embodiments, at least a portion of the first portion of tissue is included in the second portion of tissue.

Dose DOE1 can comprise a delivery of energy that reversibly alters target tissue (e.g. a volume of tissue intended to be reversibly altered by dose DOE1), while dose DOE2 can comprise a delivery of energy that irreversibly alters the target tissue (e.g. a volume of tissue intended to be irreversibly altered by dose DOE2). Changes, or lack of changes, to tissue (e.g. target tissue) shall be described herein in terms of the effects encountered by a majority, but not necessarily all, of the target tissue. For example, as used herein, “reversibly altering tissue”, and the like, can refer to a reversible altering of all, or simply a majority of tissue (e.g. a majority of target tissue), in other words, a small portion (e.g. less than 30%, 20%, or 10%) of the target tissue (e.g. tissue intended to be reversibly altered by dose DOE1) may be irreversibly altered by the delivery of the energy, or not altered at all, while the majority of the target tissue (e.g. at least 70%, 80%, or 90%, respectively) is reversibly altered. Similarly, as used herein, “irreversibly altering tissue”, and the like, can refer to an irreversible altering of all, or simply a majority of target tissue (e.g. a majority of tissue intended to be altered by dose DOE2). In other words, a small portion (e.g. less than 30%, 20%, or 10%) of the target tissue may be reversibly altered by the delivery of the energy, or not altered at all, while the majority of the target tissue (e.g. at least 70%, 80%, or 90%, respectively) is irreversibly altered.

Dose DOE1 can be configured to enhance the effect (e.g. the tissue effect) caused by dose DOE2 (e.g. when at least a portion of dose DOE2 is delivered after completion of the delivery of dose DOE1), such as is described herein.

Dose DOE1 can comprise a delivery of energy that is below a threshold, such as a threshold of delivered energy that causes the target tissue (e.g. the tissue receiving the energy and potentially some neighboring tissue) to change from an initial state (e.g. an initial temperature, pressure, level of cell membrane permeability, viability level, state of health, and/or other tissue state), and then subsequently return to that initial state over time (e.g. within 10 minutes, 1 hour, or 1 day). For example, dose DOE1 can comprise a delivery of energy that simply causes the cooling or warming of target tissue from body temperature, where the target tissue returns to body temperature within a relatively short time period after the cessation of energy delivery, such as when dose DOE1 comprises an energy delivery that is insufficient to ablate, insufficient to cause necrosis of, and/or otherwise insufficient to permanently alter the target tissue (e.g. an RF energy delivery that has an amplitude, frequency, duration, and/or other parameter that is insufficient to ablate, necrose, and/or otherwise permanently alter the target tissue). Dose DOE1 can comprise a delivery of RF energy that is delivered in a monopolar mode (e.g. between energy delivery element 130a and/or other energy delivery elements 130 of EDD 100 and return electrode 130′), and/or RF energy that is delivered in a bipolar mode (e.g. between two delivery elements 130 of EDD 100 and/or between any two elements of system 10). Dose DOE1 can comprise a delivery of non-electrical energy (e.g. light energy, ultrasound energy, and/or thermal energy) that causes a parameter change of (e.g. an increase in the temperature of) the tissue receiving dose DOE1 (e.g. the same tissue to receive dose DOE2).

Dose DOE2 can comprise a delivery of energy that is above a threshold, such as a threshold of delivered energy that causes the target tissue (e.g. the tissue receiving the energy and potentially some neighboring tissue) to change from an initial state (e.g. an initial pressure, level of cell membrane permeability, viability level, state of health, and/or other tissue state) without subsequently returning to that initial state over time (e.g. not within a time period of 4 hours, 1 week, 1 month, 3 months, 6 months, 1 year, or 2 years). For example, dose DOE2 can comprise a delivery of energy that causes an irreversible change and/or other desired long-term effect to target tissue, such as when dose DOE2 comprises an energy level that is sufficient to ablate, sufficient to cause necrosis of, and/or otherwise sufficient to permanently alter the target tissue (e.g. energy delivered in the form of an IEP dose, described herebelow, that has an amplitude, frequency, duration, and/or other parameter that is sufficient to create a desired lesion in target tissue, such as to treat AF or other arrhythmia of the patient).

Doses DOE1 and DOE2 can be delivered sequentially, such as when dose DOE2 is delivered immediately after or at least soon after the completion of the delivery of dose DOE1. In some embodiments, at least a portion of the delivery of dose DOE2 (e.g. an initial portion of dose DOE2) is delivered during at least a portion of the delivery of dose DOE1 (e.g. final portion of dose DOE1), such as in an overlapping and/or interleaving arrangement.

In some embodiments, dose DOE2 comprises a delivery of energy that causes irreversible electroporation of the target tissue. For example, dose DOE2 can comprise the delivery of an “IEP” dose. An IEP dose, as used herein, can comprise one or more electrical pulses delivered between two or more electrodes, the pulses configured to generate an electric field within tissue proximate the two electrodes. The parameters of the electrical pulses can be selected such that the resultant electric field causes the irreversible electroporation of the target tissue, while avoiding significant thermal damage to the tissue (e.g. delivery of excessive heat to tissue is avoided). For example, the IEP dose can be configured to prevent the tissue receiving the dose from exceeding a temperature of 50° C. In some embodiments, the IEP dose is configured to limit a resultant temperature increase in tissue (e.g. the tissue receiving the IEP dose), such as to limit the effect of the IEP dose to a temperature increase that does not exceed 13° C., 11° C., 9° C., or 7° C.

In some embodiments, the IEP dose is delivered by an EDD 100 with an electrode-based energy delivery element 130 with a length of at least 1.46 mm, and/or a length of no more than 8 mm. In some embodiments, the IEP dose is delivered by an EDD 100 with two electrode-based energy delivery elements 130 that are separated by at least 1 mm, and/or separated by no more than 11 mm. In some embodiments, the IEP dose is delivered based on a provided voltage (e.g. provided by EDC 300) of at least 500V, and/or no more than 5000V. In some embodiments, the IEP dose comprises a field strength of at least 200V/cm, and/or no more than 1000V/cm. In some embodiments, the IEP dose comprises a pulse width of at least 0.1 μsec, and/or no more than 200 μsec. In some embodiments, the IEP dose comprises a series of pulses with a pulse repetition interval of at least 1 μsec.

In some embodiments, an IEP dose of the present inventive concepts comprises one, two, three, or more energy delivery parameter levels selected from the group consisting of: a voltage gradient of at least 50V/cm, at least 100V/cm; at least 300V/cm, or at least 400V/cm; a voltage gradient of no more than 8000V/cm, or no more than 800V/cm; an amplitude of no more than 5000V; an amplitude of no more than 2000V; an amplitude of no more than 1000V; a set of at least 2 pulses; a set of no more than 15 pulses; a set of pulses, each of at least 1 microsecond in duration; an IEP duration of at least 5 microseconds; an IEP duration of no more than 30 seconds; and combinations thereof. In some embodiments, dose DOE2 comprises an IEP dose that is delivered between two electrode-based energy delivery elements 130 that are positioned at least 2 mm, 5 mm, 7 mm, or 10 mm apart. In some embodiments, one of the delivery elements 130 receiving and/or delivering the IEP dose is positioned at the distal end (tip) of EDD 100 (e.g. element 130a shown). In some embodiments, one or both of the delivery elements 130 receiving and/or delivering the IEP dose comprises an electrode that is circular in shape (e.g. a ring electrode). In some embodiments, the delivery elements receiving and/or delivering the IEP dose comprise a set of electrodes that are positioned on multiple devices, for example a first energy delivery element 130 positioned on EDD 100, and a second energy delivery element, positioned on another device of system 10 (e.g. mapping catheter 200 and/or a second EDD 100 configured as a guidewire).

In some embodiments, dose DOE2 comprises an IEP dose, and dose DOE1 comprises a delivery of energy (e.g. RF energy) that warms the target tissue (e.g. tissue warming that is performed prior to delivery of dose DOE2). The warming caused by dose DOE1 can provide one or more benefits, such as: a reduction in the required amplitude of the IEP dose of dose DOE2 to achieve successful energy delivery (achieve a successful lesion creation); a decrease in the duration of the IEP dose; a modification of the frequency of the IEP dose; a modification of the waveform shape of the IEP dose; and/or improve the efficacy (ablative effects) of the IEP dose. In some embodiments, dose DOE1 is configured to cause the tissue receiving the dose to increase at least 2° C., such as at least 3° C. or at least 4° C.

In some embodiments, dose DOE2 comprises an IEP dose that is delivered by one or more pairs of electrode-based energy delivery elements 130, such as when one of the elements 130 of each pair is configured as a cathode, and the other is configured as an anode. System 10 (e.g. EDC 300) can be configured (e.g. via algorithm 335 described herein) to select which elements 130 are to deliver the IEP dose (e.g. which pair of a set of three or more elements 130), as well as which element 130 is to be the cathode and which is to be the anode. In some embodiments, a tip-positioned element 130 (e.g. element 130a of FIG. 1, positioned on the distal end of shaft 110) is configured as the cathode, and a more proximal element 130 (e.g. a ring electrode, such as one or more of the elements 130b-d of FIG. 1) is configured as the anode. In some embodiments, dose DOE1 and/or DOE2 comprises delivery of electrical energy between a pair of electrodes comprising one or more elements 130 configured as an anode, and one or more elements 130 configured as a cathode. For example, system 10 (e.g. one or more EDD's 100) can comprise multiple energy delivery elements 130 configured to function as an anode, a cathode, or both.

Dose DOE2 can comprise an IEP dose that is delivered while the impedance of the tissue receiving the dose is monitored by system 10, such as when system 10 delivers the IEP dose in a closed loop arrangement (e.g. based on impedance level), and/or when successful irreversible electroporation of the target tissue is confirmed via the impedance measurement (e.g. and system 10 automatically stops the delivery of the IEP dose upon the confirmation).

Doses DOE1 and/or DOE2 can be delivered by one or more energy delivery elements 130 to one or more types of target tissue, such as cardiac tissue (e.g. myocardial tissue or “myocardium”), nerve tissue, vessel wall tissue, and/or any organ tissue. In some embodiments, doses DOE1 and/or DOE2 can be configured to be delivered to tissue selected from the group consisting of: cardiac tissue; nerve tissue; vessel wall tissue; organ tissue; brain tissue; lung tissue; kidney tissue; liver tissue; stomach tissue; muscle tissue; and combinations thereof. Doses DOE1 and/or DOE2 can be delivered to the surface of an organ (e.g. an endocardial and/or epicardial surface of the heart), and/or within solid tissue of an organ (e.g. beneath a surface, such as within heart wall tissue and/or beneath the surface and within solid tissue of another type of organ). In some embodiments, doses DOE1 and/or DOE2 are configured to avoid adversely affecting blood, or other tissue deemed to be non-target tissue.

EDC 300 can comprise an energy delivery module, module 360 shown, such as an energy delivery module configured to provide ablation energy to EDD 100 (e.g. provide energy of doses DOE1 and DOE2 and/or other energy to one or more energy delivery elements 130 comprising one or more electrodes and/or other energy delivery elements). Energy delivery module 360 can provide energy to EDD 100 via a patient interface unit, PIU 310 (as shown and described herein), or otherwise. As described herein, energy provided by module 360 can comprise an energy form selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy (e.g. as delivered by a pharmaceutical drug or other agent); and combinations of these. Energy delivery module 360 can comprise an energy delivery module selected from the group consisting of: RF generator; light energy delivery unit; cryogenic energy delivery unit; ultrasound energy delivery unit; microwave energy delivery unit; electroporation energy delivery unit; and combinations of these. In some embodiments energy delivery module 360 comprises an RF generator configured to provide RF ablation energy to one or more energy delivery elements 130 (i.e. when each energy delivery element 130 comprises an electrode). Doses DOE1 and DOE2 can comprise similar or dissimilar forms of energy (e.g. RF energy and another form of energy).

In some embodiments, EDC 300 comprises one or more functional elements, such as functional element 309 shown and described herein.

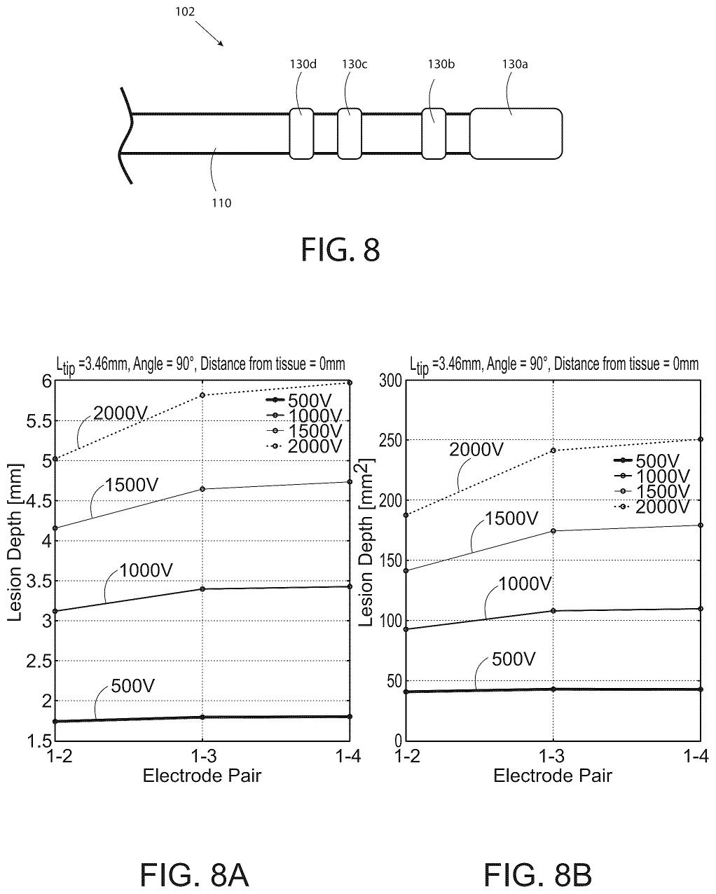

EDD 100 can comprise one or more devices that are configured to deliver energy, such as one, two, or more energy-delivering devices that each comprise a catheter, surgical tool, laparoscopic tool, and/or endoscopic tool. EDD 100 can include shaft 110, typically a flexible shaft, including proximal end 111. EDD 100 includes distal portion 102 shown. An operator graspable portion, handle 120, can be positioned on proximal end 111 of shaft 110. Handle 120 can comprise one or more controls (e.g. one or more buttons, switches, levers, and the like), such as control 121 shown. In some embodiments, EDD 100 distal portion 102 is of similar construction and arrangement as the distal portion of EDD 100 of FIG. 8 described herein.

EDD 100 comprises one or more elements configured to deliver energy to tissue, such as energy delivery elements 130a-d shown in FIG. 1. In some embodiments, one or more energy delivery elements 130 is configured to deliver a first dose of energy, dose DOE1 (e.g. as provided by EDC 300 and as described herein, such as RF energy delivered in a monopolar or bipolar arrangement), and a pair of energy delivery elements 130 is configured to deliver a second dose of energy, dose DOE2 (e.g. also as provided by EDC 300 and as described herein). In some embodiments, dose DOE1 and dose DOE2 are delivered by the same set of components (e.g. the same pair of elements 130). Alternatively, energy delivery element 130 used to deliver dose DOE1 is not included in the set of elements 130 used to deliver dose DOE2, or vice versa. In some embodiments, dose DOE1 is delivered by one or more energy delivery elements 130 (e.g. at least element 130a), and dose DOE2 is delivered by at least two energy delivery elements 130 (e.g. at least element 130a and one or more of elements 130b-d).

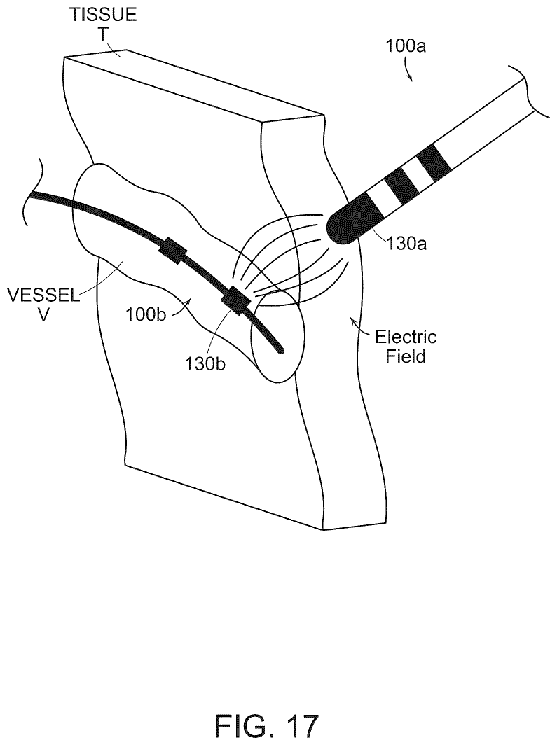

Each energy delivery element 130 can comprise one or more elements configured to deliver one, two or more forms of energy selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy; and combinations of these. In some embodiments, at least one energy delivery element 130 is configured to deliver at least two forms of energy selected from the group consisting of: thermal energy, such as heat energy or cryogenic energy; electromagnetic energy, such as radiofrequency (RF) energy and/or microwave energy; light energy, such as light energy provided by a laser; sound energy, such as subsonic energy or ultrasonic energy; chemical energy; and combinations of these. Energy delivery element 130 can comprise one or more energy delivery elements positioned on the distal portion of EDD 100, device distal portion 102 shown. Energy delivery element 130 can include at least one energy delivery element (e.g. at least one electrode, at least one optical element configured to deliver light energy, and/or at least one cryogenic fluid delivery element) positioned on the distal end of EDD 100 in a “tip electrode” configuration. In some embodiments, EDD 100 can comprise two, three, or more energy delivery elements 130, such as multiple electrodes configured to deliver monopolar and/or bipolar electromagnetic (e.g. RF) energy to heat, ablate, and/or otherwise therapeutically affect target tissue (e.g. create a desired lesion in target tissue). One or more energy delivery elements 130 can each comprise an electrode, such as an electrode configured to deliver radiofrequency (RF) and/or other electromagnetic energy. Two or more energy delivery elements 130 can be configured as a pair of electrodes that delivers an irreversible electroporation pulse of energy (e.g. as provided as dose DOE2 by EDC 300 as described herein). Energy delivery elements 130 can comprise one or more electrodes positioned at the end of EDD 100 (e.g. element 130a shown in FIG. 1). Energy delivery elements 130 can comprise an array of energy delivery elements (e.g. an array of electrodes), such as is shown in FIGS. 1 and 8. In some embodiments, energy delivery element 130 comprises a return electrode pad, electrode 130′ shown in FIG. 1. Electrode 130′ can comprise an electrode configured as a return electrode for delivery of energy between one or more elements 130 of EDD 100 (e.g. for delivery of monopolar RF energy by EDD 100). In some embodiments, two, three, four, or more energy delivery elements 130 configured as a pair (e.g. a bipolar energy delivery pair) can be positioned on separate devices, for example a first energy delivery element 130 positioned on EDD 100 and a second energy delivery element 130 positioned on a second EDD 100 configured as a guidewire (e.g. a second EDD 100 that is positioned within an epicardial vessel, such as described herebelow in reference to FIG. 17).

In some embodiments, EDD 100 comprises two or more devices for delivering energy to tissue, such as a first EDD 100′ comprising one or more energy delivery elements 130, and a second EDD 100″ comprising one or more energy delivery elements 130 (EDD 100′ and 100″ not shown, but similar or dissimilar energy delivery devices each comprising one or more delivery elements 130). In these embodiments, dose DOE1 and/or dose DOE2 can comprise a dose that is delivered between an element 130 of EDD 100′ and an element 130 of EDD 100″. For example, an RF energy dose and/or an IEP dose can be delivered between an element 130 of EDD 100′ that is positioned at a location on an endocardial surface of the heart, and an element 130 of EDD 100″ that is positioned at a location on an epicardial surface of the heart (e.g. an epicardial surface location that is relatively close to the endocardial surface location of element 130 of EDD 100′).

EDD 100 can comprise an assembly configured to measure, monitor, react to, and/or maintain a force (e.g. a force between tissue and one or more portions of EDD 100), such as force maintenance assembly 150 shown. Force maintenance assembly 150 can be positioned within handle 120, within a portion of shaft 110 (e.g. within the distal portion 102 of EDD 100), and/or on the distal end of shaft 110 (e.g. within distal portion 102 of EDD 100 as shown). Force maintenance assembly 150 can comprise one or more elements configured to provide or maintain a force, force maintenance elements 160 shown and as described herein. As examples, such force maintenance elements 160 can be or can include one or more of: a hydraulic element, a spring, a magnet, a compressible fluid, a memory material, and the like. The force maintenance elements 160 can be located at a distal end, proximal end, or intermediate portion of EDD 100, or a combination of two or more thereof. Force maintenance assembly 150 can also comprise one or more sensing elements, sensing elements 158 shown, which can take the form of and/or can include one or more sensors (e.g. proximity sensors). In some embodiments, force maintenance assembly 150 comprises a similar construction and arrangement, and similar components, as described in applicant's co-pending U.S. patent application Ser. No. 16/335,893, titled “Ablation System with Force Control”, filed Mar. 22, 2019.

Force maintenance assembly 150 can be axially aligned with shaft 110 (e.g. a major axis of force maintenance assembly 150 is aligned with a central axis of distal portion 102), such as when assembly 150 is aligned with distal portion 102. Force maintenance assembly 150 can be configured to absorb mechanical shocks. Alternatively or additionally, force maintenance assembly 150 can be configured to dynamically (e.g. dynamically and automatically) respond to movement of the heart wall or other cardiac tissue onto which one or more energy delivery elements 130 are positioned to deliver energy (e.g. avoiding reliance on the clinician to manually react to the movement of the endocardial surface in a cardiac ablation procedure). The force maintenance assembly 150 can allow and/or compensate for high and/or low frequency movements, various movement ranges, and the like. Force maintenance assembly 150 can be configured to compress over a “travel distance” (also referred to as the “compression distance” and equal to the distance force maintenance assembly 150 compresses when a force is applied) up to a pre-determined maximum distance (the “max compression distance” or “max travel distance”), such as a maximum distance comprising a length between 0.1 mm to 10 mm, a maximum distance comprising a length between 0.1 mm and 5 mm, and/or some other predetermined distance range and/or limit.

Force maintenance assembly 150 can be configured to provide a pre-determined force range over all or a portion of the travel distance, for example a pre-determined constant and/or variable force (e.g. a force between 0.1 gmf and 100 gmf, between 5 gmf and 30 gmf, and/or between 10 gmf and 30 gmf). In some embodiments, force maintenance assembly 150 is configured to provide a relatively constant force over all or a portion of the travel distance, for example a pre-determined constant force between 0.1 gmf and 100 gmf, such as between 5 gmf and 30 gmf, or between 10 gmf and 30 gmf. Additionally or alternatively, in some embodiments force maintenance assembly 150 is configured to provide a variable force over all or a portion of the travel distance, such as a variable force that varies within a pre-determined range of forces (e.g. a range of forces proportional to the amount compressed). For example, force maintenance assembly 150 can be configured to apply a force that varies between 5 gmf and 30 gmf, such as a force that varies between 10 gmf and 30 gmf.

As described hereabove, force maintenance assembly 150 can include one or more sensing elements or sensors, such as sensing element 158 shown, which can be configured to produce a signal correlating to the amount of compression of force maintenance assembly 150. Additionally or alternatively, sensing element 158 can be configured to produce a signal correlating to maximum compression of force maintenance assembly 150 (e.g. a maximum force achieved during compression).

Energy delivery element 130 can be positioned on the distal end of shaft 110, such as when force maintenance assembly 150 is positioned within shaft 110. Alternatively, energy delivery element 130 can be positioned on the distal end of the force maintenance assembly 150.

EDD 100 can be configured for ablation of an atria of the heart (e.g. to create one or more lesions to treat atrial fibrillation or right atrial flutter) and/or for ablation of a ventricle of the heart (e.g. to treat ventricular tachycardia). For ablation of an atria, force maintenance assembly 150 can be configured with a first max compression distance, such as a distance less than or equal to 10 mm, less than or equal to 5 mm, or less than or equal to 3 mm. Alternatively, for ablation of a ventricle, force maintenance assembly 150 can be configured with a second max compression distance, such as a distance greater than the first max compression distance, such as a distance at least 1 mm greater than the first max compression distance, such as a second (ventricular) max compression distance of at least 3 mm or at least 6 mm. In some embodiments, the first (atrial) max compression distance comprises a distance of approximately 2-3 mm. In some embodiments, the second (ventricular) max compression distance comprises a distance of approximately 4-6 mm.

System 10 can include at least a second energy delivery device, EDD 100′, such as a second EDD 100′ that is configured for use in an atria and/or a ventricle of the heart (e.g. EDD 100′ comprises a catheter for insertion into the patient's vascular system and into a chamber of the patient's heart). In some embodiments, first EDD 100 is configured for use in an atria (e.g. and not a ventricle) and second EDD 100′ is configured for use in a ventricle (e.g. and not an atria). In these embodiments, first EDD 100 can include a force maintenance assembly 150 comprising a shorter max compression distance as compared to the max compression distance of the force maintenance assembly 150 positioned within second EDD 100′. In some embodiments, dose DOE2 comprises an IEP pulse configured to create an effective lesion in a ventricle of the heart of the patient, such as when the IEP dose is based on a voltage of no more than 5 kV. In some embodiments, dose DOE2 comprises an IEP pulse configured to create an effective lesion in an atrium of the heart of the patient, such as when the IEP dose is based on a voltage of no more than 2 kV.

EDD 100 can comprise one or more electrodes, mapping electrodes 135 shown, which can be configured to record biopotential information (e.g. cardiac electrical activity data and the like) and/or to record position information (e.g. data regarding EDD 100 position within the patient's anatomy). Mapping electrode 135 can comprise one or more electrodes positioned on distal portion 102 of EDD 100, as shown. Mapping electrode 135 can comprise a ring electrode. In some embodiments, mapping electrode 135 comprises at least one sensor or sensing element (“sensor” herein), such as an electrode-based sensor and/or a non-electrode-based sensor (e.g. a light sensor, a temperature sensor, a pH sensor, a physiologic sensor such as a blood sensor, a blood gas sensor, and the like). In some embodiments, one or more mapping electrodes 135 and one or more energy delivery elements 130 comprise the same component. In some embodiments, one or more mapping electrodes 135 can be configured as a return electrode (e.g. a return electrode for an IEP pulse).

EDD 100 is configured to operably attach to EDC 300. EDD 100 comprises one or more wires, filaments, and/or other conduits, conduit 125, and one or more attached connectors, connector 126. Connector 126 operably attaches (e.g. at least electrically attaches) to a mating connector, connector 301b of EDC 300. Conduit 125 can comprise one or more wires or conductive traces (“wires” herein), optical fibers, tubes (e.g. hydraulic, pneumatic, irrigation or other fluid delivery tubes), wave guides, and/or mechanical linkages (e.g. translating filament), each of which can be used to operably attach one or more components of EDC 300 to one or more components of EDD 100.

System 10 can comprise one or more functional elements, such as functional elements 119, 129, 219, 229, and/or 309 shown in FIG. 1 and described in detail herein. Functional elements 119, 129, 219, 229, and/or 309 can each comprise one or more sensors and/or one or more transducers, as described herein. In some embodiments, functional elements 119, 129, 219, 229, and/or 309 comprise a transducer selected from the group consisting of: heating element; cooling element; vibrational transducer; ultrasound transducer; electrode; light delivery element; drug or other agent delivery element; and combinations of one or more of these. In some embodiments, functional elements 119, 129, 219, 229, and/or 309 comprise a sensor selected from the group consisting of: a physiologic sensor; a blood pressure sensor; a blood gas sensor; a pressure sensor; a strain gauge; a force sensor; a chemical sensor; an impedance sensor; a magnetic sensor; an electrode; a displacement sensor (e.g. a sensor configured to determine the distance force maintenance assembly 150 is compressed); a flow sensor; and combinations of one or more of these. In some embodiments, functional elements 129 and/or 229 comprise functional elements configured to provide feedback, and/or otherwise alert the user to the status of one or more components of system 10 (e.g. when an undesired condition is present). Functional elements 129 and/or 229 can comprise an element selected from the group consisting of: a haptic transducer; a light source, such as an LED light source; an audio transducer, such as a speaker; and combinations of one or more of these.

Mapping catheter 200 of system 10 includes shaft 210, typically a flexible shaft comprising one or more lumens. Positioned on distal end 213 as shown, or positioned at least on a distal portion of shaft 210, is basket assembly 230. An operator graspable portion, handle 220, is positioned on proximal end 211 of shaft 210. Handle 220 can comprise one or more controls, such as control 221 shown.

Basket assembly 230 can comprise an expandable assembly, such as an assembly resiliently biased in a radially expanded or compacted state, and configured to correspondingly be compacted or expanded, respectively, such as via control 221, by advancing out of the distal end of a sheath (to radially expand), and/or by being retracted within a sheath (to radially compact), such as sheath 12 or the like. Basket assembly 230 comprises an array of filaments, splines 231, which can comprise metal (e.g. stainless steel and/or nickel titanium alloy) and/or plastic filaments that are resiliently biased (e.g. biased in an expanded and/or compacted state). Basket assembly 230 can include a plurality of electrodes, electrodes 232, which are coupled to splines 231. Additionally or alternatively, basket assembly 230 can include a plurality of ultrasound transducers, transducers 233 which can also be coupled to splines 231. In some embodiments, basket assembly 230 and/or mapping catheter 200 are of similar construction and arrangement to the similar components described in applicant's co-pending U.S. patent application Ser. No. 17/673,995, titled “Device and Method For the Geometric Determination of Electrical Dipole Densities on the Cardiac Wall”, filed Feb. 17, 2022, and/or applicant's co-pending U.S. patent application Ser. No. 16/242,810, titled “Expandable Catheter Assembly with Flexible Printed Circuit Board (PCB) Electrical Pathways”, filed Jan. 8, 2019. In some embodiments, one or more electrodes 232 and/or ultrasound transducers 233 additionally or alternatively include a sensor, such as a physiologic sensor and/or other sensor, such as are described herein.

Mapping catheter 200 of system 10 can comprise one or more functional elements, such as functional elements 219, 229 shown, and described herein. In some embodiments, one or more functional elements 219 and/or 229 are positioned on basket assembly 230 (e.g. on one or more splines 231).

Mapping catheter 200 can be configured to operably attach to EDC 300. Mapping catheter 200 can comprise one or more wires, filaments, and/or other conduits, conduit 225, and one or more attached connectors, connector 226, each as shown. Connector 226 operably attaches (e.g. at least electrically attaches) to a mating connector, connector 301a of EDC 300. Conduit 225 can comprise one or more wires, optical fibers, tubes (e.g. hydraulic, pneumatic, irrigation or other fluid delivery tubes), wave guides, and/or mechanical linkages (e.g. translating filament), each of which can be used to operably attach one or more components of EDC 300 to one or more components of mapping catheter 200. In some embodiments, EDC 300 comprises two, three, or more interconnected discrete components, each configured to operably attach to one or more other components of system 10 (e.g. EDD 100 and/or mapping catheter 200). For example, EDC 300 can include a first component configured to operably attach to mapping catheter 200 and perform one or more mapping functions of system 10 described herein, and a second component configured to operably attach to EDD 100 and to deliver energy to EDD 100, as described herein. These separate components can be operably connected to each other (such as via ethernet or other data and/or power transfer mechanism), and/or each component can be operably connected to a third component configured to provide an interface between the first and second components.

System 10 can include one or more patch electrodes, patch 60 shown, which can comprise standard skin electrodes and/or other electrodes configured to attach (e.g. adhesively attach) to the skin of the patient and transmit electrical signals through the patient and/or receive electrical signals from the patient. In some embodiments, patches 60 are configured to record the patient's electrocardiogram (ECG) and/or to transmit and/or receive localization signals of system 10. Each patch 60 can be configured to operably attach (e.g. at least electrically attach) to EDC 300. Each patch 60 can comprise one or more conduits, conduit 65, (e.g. one or more electrical wires), and one or more attached connectors, connector 66. Connector 66 operably attaches to a mating connector, connector 301c of EDC 300.

EDC 300 includes one or more internal components configured to control and/or otherwise interface with the one or more energy delivery devices, EDD 100, one or more mapping catheters 200, and/or one or more patches 60. EDC 300 comprises one or more wires, filaments, and/or other conduits, conduits 302, such as conduits 302a, 302b, and/or 302c, which via connectors 301a, 301b, and/or 301c operably connect (e.g. at least electrically connect) to the one or more EDDs 100, one or more mapping catheters 200, and/or one or more patches 60, respectively. Conduits 302 can comprise one or more wires, optical fibers, tubes (e.g. hydraulic, pneumatic, irrigation or other fluid delivery tubes), wave guides, and/or mechanical linkages (e.g. translating filament).

EDC 300 can comprise a patient interface unit, PIU 310. PIU 310 can be connected (e.g. at least electrically connected) to one or more of components 320, 330, 340, 350, 360, and/or 370, each described in detail herein, for example via bus 305 shown. Bus 305 can comprise one or more wires, optical fibers, and/or other conduits configured to provide power, transmit data, and/or receive data. In some embodiments, bus 305 comprises one or more fluid delivery tubes configured to provide hydraulic fluid, irrigation fluid, and/or other fluid as described herein. PIU 310 can be operably attached to components 340, 350, 360, and/or 370, such as to allow power, data, fluids, and/or mechanical linkages to pass between PIU 310 and one or more of: EDD 100, mapping catheter 200, and/or patches 60. In some embodiments, PIU 310 can reduce undesired electrical interaction between two or more modules of EDC 300. For example, PIU 310 can include one or more filters (e.g. one, two or more parallel LC notch filters and/or low-pass filters) configured to reduce electrical interference between a mapping module (e.g. mapping module 350 or other mapping module of system 10) and an RF generator (e.g. energy delivery module 360 or other electromagnetic energy delivery module of system 10), such as interference from signals transmitted into and received from the patient. PIU 310 can include one or more components selected from the group consisting of: a filter; a transformer; a buffer; an amplifier; a pass thru (e.g. a conduit that is unfiltered or otherwise unaltered by PIU 310, such as a fluid conduit); and combinations of one or more of these. In some embodiments, PIU 310 comprises an electrical protection circuit configured to protect EDC 300 from damage caused by high-energy signals such as defibrillation pulses and/or RF ablation energy delivered to the patient.

EDC 300 can include one, two, or more interfaces, user interface 320 shown, and each interface 320 can be configured for providing and/or receiving information to and/or from a user of system 10. User interface 320 can include one, two, or more user input and/or user output components. For example, user interface 320 can comprise a joystick, keyboard, mouse, touchscreen, and/or another human interface device, user input device 321 shown. In some embodiments, user interface 320 comprises a display, such as display 322, also shown. Processor 330 can provide a graphical user interface, GUI 325, to be presented on display 322.

EDC 300 can comprise a signal processing assembly, processor 330. Processor 330 can comprise at least one microprocessor, computer, and/or another electronic controller. In some embodiments, processor 330 comprises a controller, a memory coupled to the controller, and one, two, or more algorithms, algorithm 335 shown. The memory of processor 330 can be configured to store instructions for the controller to perform the algorithm 335. Processor 330, via algorithm 335, can perform (e.g. guide and/or otherwise control the performance of) one or more of the processes and/or functions of system 10 described herein. Processor 330 can provide a graphical user interface, GUI 325, to be presented on display 322. In some embodiments, processor 330 is configured to perform a process in response to one or more commands that the user inputs into system 10 via GUI 325. Processor 330 can receive a signal, such as a signal from one or more sensors (as described herein) of EDD 100 and/or mapping catheter 200. Processor 330 can be configured to perform one or more mathematical operations on the received signal, and to produce a result correlating to a quantitative or qualitative measure of the force applied by EDD 100 to tissue, the amount of compression of force maintenance assembly 150, the orientation of EDD 100, the proximity of a portion of EDD 100 to cardiac tissue, and/or the level or quality of contact between a portion of EDD 100 and cardiac tissue. The one or more mathematical operations can comprise an operation of function selected from the group consisting of: arithmetic operations; statistical operations; linear and/or non-linear functions; operations as a function of time; operations as a function of space or distance; comparison to a threshold; comparison to a range; and combinations of one or more of these. In some embodiments, algorithm 335 comprises a machine learning or other artificial intelligence (AI) algorithm. In some embodiments, algorithm 335 is configured to monitor, assess and/or control (“control” herein) force maintenance assembly 150 (e.g. adjust one or more parameters of force maintenance assembly in a closed loop or semi-closed loop arrangement), such as control based on the sensor signal. In some embodiments, algorithm 335 is configured to determine and/or assess at least one of contact, force, or pressure applied by EDD 100 to tissue (e.g. target tissue). In some embodiments, algorithm 335 processes one or more signals received from one or more sensors of system 10, such as a signal correlating to: the temperature of the energy delivery element; the temperature of the tissue surrounding and/or otherwise proximate the energy delivery element; the duration of energy delivery to tissue; the level of energy being delivered to tissue; the force and/or pressure being applied to tissue; and combinations of one or more of these. Algorithm 335 can be configured to modify the energy delivery based on these signals, for example to stop the energy delivery when a combination of sufficient parameter levels has been reached, for example when a sufficient energy delivery at a sufficient pressure for a sufficient period of time has been reached. In some embodiments, system 10 is configured to deliver increased energy levels to decrease duration of energy delivery to tissue (e.g. target tissue). Alternatively or additionally, system 10 can be configured to increase duration of energy delivery to tissue, such as to cause a corresponding decrease in an energy level of the energy delivery. In some embodiments, system 10 controls force (e.g. in a closed loop manner) between one or more energy delivery elements 130 and tissue to adjust one or more of duration of energy delivery and/or level of energy delivery (e.g. voltage level, current level and/or power level). In some embodiments, system 10 adjusts duration of energy delivery and/or level of energy delivery based on a measured and/or controlled level of force between one or more energy delivery elements 130 and tissue.

In some embodiments, algorithm 335 (e.g. an AI algorithm) is configured to define, adjust, and/or otherwise control the delivery of energy by EDC 300 to EDD 100, such as to control dose DOE1 and/or DOE2. In some embodiments, algorithm 335 is configured to modify (e.g. in a closed loop arrangement) the delivery of dose DOE1 and/or DOE2, such as to perform a modification based on tissue impedance and/or other physiologic parameter of the patient. In some embodiments, algorithm 335 is configured to modify dose DOE2 based on a parameter (e.g. a measured parameter) associated with a previously delivered dose DOE1.

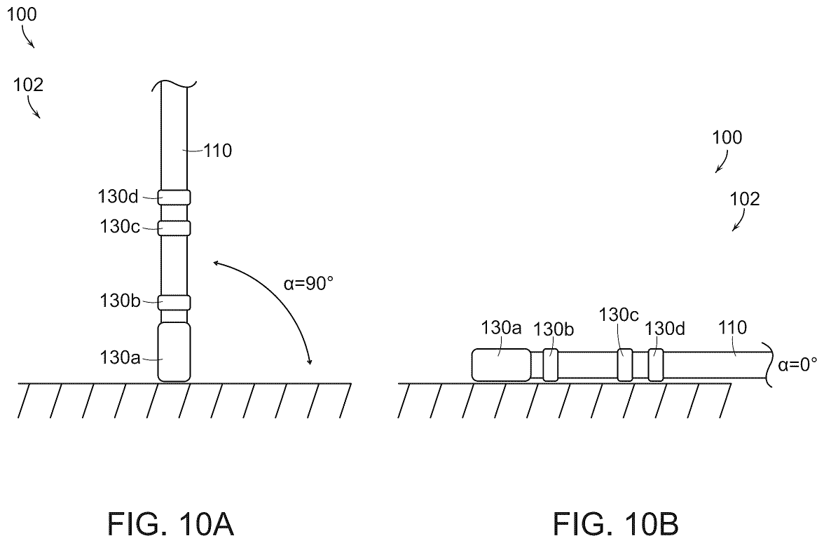

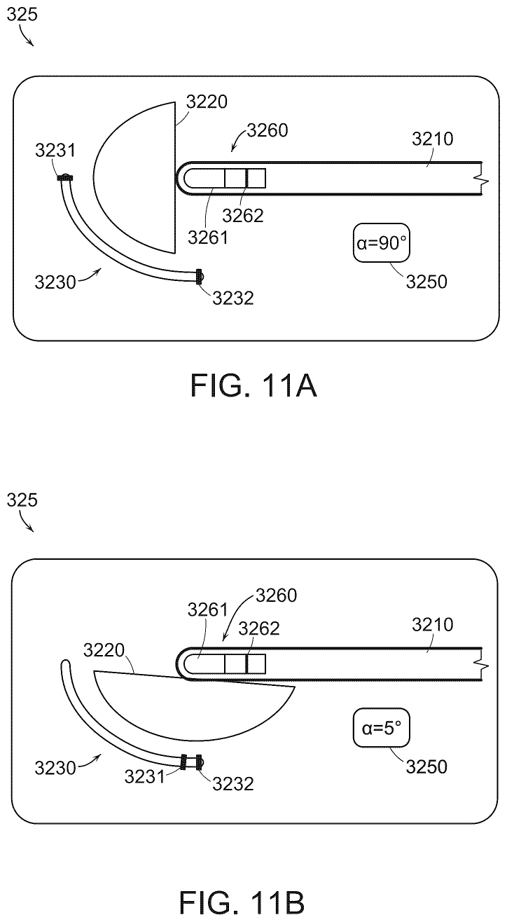

Algorithm 335 can be configured to determine the orientation angle of an EDD 100, such as a determination based on data provided by a sensor of system 10, and/or by a separate imaging device, as described herein in reference to FIGS. 10-11.

Algorithm 335 can be configured to determine electric field strength, such as to determine the field strength of an electric field used to perform pulsed field ablation, such as is described herein in reference to FIGS. 10-13.

Algorithm 335 can be configured to provide lesion information, such as a predicted size (e.g. length, width, depth, and/or volume) of a lesion to be created, such as is described herein in reference to FIGS. 10-13.

EDC 300 can comprise a fluid delivery module, module 370 shown, which can be configured to deliver a fluid (e.g. a hydraulic fluid and/or an irrigation fluid as described herein), to EDD 100 and/or mapping catheter 200, such as via PIU 310 as shown. In an alternative embodiment, fluid delivery module 370 is connected to EDD 100 and/or mapping catheter 200 without passing through PIU 310. Fluid delivery module 370 can comprise one or more fluid delivery devices (e.g. peristaltic pump, syringe pump, gravity-feed flow controller and/or other fluid propulsion device) which can be attached to one or more sources of saline and/or other fluid, fluid 70 shown.

Fluid 70 can comprise a fluid of a known conductivity (e.g. a relatively low conductivity and/or a conductivity at least less than that of blood), such as a fluid delivered to steer current and/or steer an electromagnetic field (e.g. to surround one or more electrodes, such as during the delivery of energy to cause pulsed field ablation of target tissue).

EDC 300 can comprise a force maintenance module, module 340 shown. Force maintenance module 340 can be configured to provide a signal that allows system 10 to adjust force applied by EDD 100 to tissue, such as to provide a control signal to force maintenance assembly 150. In some embodiments, force maintenance module 340 is configured to deliver and/or at least control (e.g. control the pressure of) a supply of hydraulic fluid to EDD 100 (e.g. via fluid delivery module 370), such as in a closed loop arrangement.

In some embodiments, force maintenance module 340 is configured to automatically adjust force between one or more electrodes 130 and tissue, such as to create a desired electric field during pulsed field ablation of target tissue.

EDC 300 can comprise a mapping module, module 350 shown. In some embodiments, mapping module 350 comprises a module configured to record and/or process ultrasound information, such as ultrasound module 351 shown. In some embodiments, mapping module 350 comprises a module configured to record and process biopotential information, such as biopotential module 352 shown. Mapping module 350 can transmit energy and/or signals to EDD 100, mapping catheter 200, and/or patches 60 via PIU 310 (as shown), or otherwise. Mapping module 350 can be configured to transmit one or more signals into the patient (e.g. via one or more patches 60), such as to create a localization field within the patient. Furthermore, mapping module 350 can receive signals from one or more electrodes (or other sensors) of EDD 100 and/or mapping catheter 200, such as signals correlating to the localization signals, such as to determine the localization of the one or more electrodes within the localization field (e.g. to determine the location and/or orientation of the associated catheter(s) within the patient). In some embodiments, two or more localization fields can be used simultaneously. The components used to generate and/or sense the localization fields (e.g. patches 60 and/or the one or more electrodes of EDD 100 or mapping catheter 200), can be configured to transmit localization signals (herein “source”), receive localization signals (herein “sink”), and/or transmit and receive localization signals interchangeably. For example, the components can be multiplexed to source and sink localization signals between each other in a pattern configured to enhance the localization information received by mapping module 350, such as information regarding the relative position between a component of system 10 and the cardiac tissue or other structures within the cardiac chamber and/or another component of system 10.

For example, the direction of current flow between two or more components used to perform a localization measurement can be reversed. For example, in an impedance-based system, multiple (e.g. 3 or 4) localization fields can be generated simultaneously, such as by using multiple frequency ranges. All electrodes and/or sensors within the field can be used to sense the localization field. The components used to source (e.g. transmit the localization signals) and sink (e.g. sense the localization signals) the localization fields can be fixed and static, such as when patches 60 positioned on the body surface are used to source the localization fields, and electrodes located on one or more components of system 10 and positioned within the patient that are used to sink the localization signals. Alternatively, the components can be time-multiplexed and/or frequency-multiplexed, such as by sourcing and sinking current from different sets of components at various frequencies and/or at various times. As an example of a time-multiplexed localization method, system 10 can include three source/sink components, A-C. In a first configuration, component A is used to source, and component B is used to sink. In a second configuration, B can be used to source and A to sink. In a third configuration, C is used to source, and B is used to sink. These three configurations can be multiplexed to provide an enhanced localization method. Using all possible permutations would provide the full complement of information available via the source-sink configurations. Subsets of these configurations can be selected to reduce electronic and algorithmic complexity, while providing sufficient information to resolve the number of conditions and/or states required. In some embodiments, the electronics are configured to minimize current leakage (e.g. paths to a ground) within a range of frequencies (such as 10-100 kHz) via sensors and/or electrodes present within the localization field and/or used to measure the localization field. For example, current leakage can be minimized with the design of a sufficiently high input impedance in the localization frequency range of interest.

In some embodiments, ultrasound module 351 of mapping module 350 is configured to transmit and receive ultrasound signals via one or more ultrasound transducers 233 of mapping catheter 200, such as to determine the distance between ultrasound transducers 233 and cardiac tissue, such as to, in coordination with the localization data, generate an anatomical model of the cardiac tissue. Biopotential module 352 of mapping module 350 can be configured to record one or more biopotential signals, such as via electrodes 232 of mapping catheter 200, to create an electrical activity map of a cardiac chamber. In some embodiments, mapping module 350, including ultrasound module 351 and biopotential module 352, are of similar construction and arrangement to the similar components described in applicant's co-pending U.S. patent application Ser. No. 17/735,285, titled “Ultrasound Sequencing System and Method”, filed May 3, 2022, and/or applicant's co-pending U.S. patent application Ser. No. 16/849,045, titled “Localization System and Method Useful in the Acquisition and Analysis of Cardiac Information”, filed Apr. 15, 2020.

One or more sensors of EDD 100 (e.g. one or more of functional elements 119 or 129 configured as one or more sensors, and/or other sensors as described herein) can be configured to produce a signal correlating to a level of contact between one or more energy delivery elements 130 and tissue (e.g. cardiac tissue). The signal provided can simply differentiate a minimum (sufficient) level of contact versus an insufficient level of contact (e.g. a lack of contact), and/or it can provide data that differentiates various levels of contact (e.g. a quantitative assessment of force between one or more energy delivery elements 130 and tissue). EDC 300 can provide qualitative and/or quantitative contact information to a user (e.g. a clinician), such as via display 322, the information indicative of the level of contact between one or more energy delivery elements 130 and tissue (e.g. chamber wall and/or other cardiac tissue). In some embodiments, system 10 is configured to provide (via display 322) information comprising: notation of sufficient contact achieved (e.g. sufficient contact to perform an efficacious delivery of energy to target tissue); notation of insufficient contact achieved; level of force achieved; level of pressure achieved; distance or proximity to a boundary; orientation or angle-of-attack to a boundary or other tissue location; topology of a proximate boundary; contact efficiency level; and combinations of one or more of these.

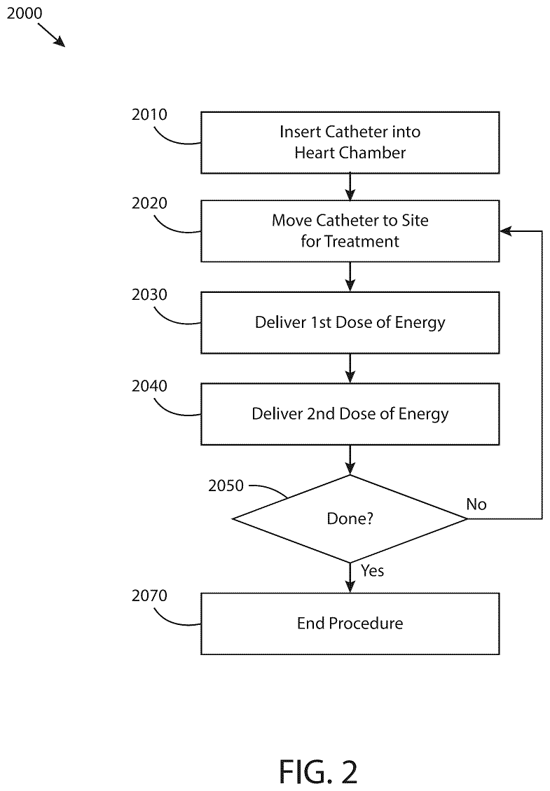

The tissue treatment methods described herebelow in reference to FIGS. 2-7 are described in reference to a catheter, such as energy delivery device EDD 100 described herein, delivering two forms of energy to target tissue of the patient. It should be considered within the spirit and scope of this application that other types of energy delivery devices could be used, such as surgical tools, laparoscopic tools, endoscopic tools, and/or other energy delivery tools. The methods described herebelow in reference to FIGS. 2-7 are described in reference to target tissue comprising heart tissue, such as heart chamber tissue in which energy is delivered to an endocardial surface of the heart. It should be considered within the spirit and scope of this application that energy can alternatively be delivered: within solid tissue of a heart wall (e.g. energy delivered from a coronary vessel) and/or to an epicardial surface; and/or to any solid tissue surface or tissue surface. Doses of energy dose DOE1 and dose DOE2 described in reference to FIGS. 2-7 can comprise similar forms of energy (e.g. where both comprise RF energy delivery), or different forms of energy (e.g. where dose DOE2 comprises RF or other electromagnetic energy delivery and dose DOE1 comprises non-electromagnetic energy delivery). Dose DOE1 can comprise a delivery of energy configured to reversibly warm target tissue, while dose DOE2 can comprise a delivery of energy configured to irreversibly electroporate the target tissue, such as via delivery of an IEP as described herein, such as to create a desired lesion in target tissue (e.g. to treat AF or other arrhythmia of the patient). This pre-warming of tissue can provide numerous advantages, as described herein, such as when dose DOE2 comprises a delivery of energy (e.g. RF energy) with a lower amplitude than that which would have been necessary to irreversibly electroporate the target tissue if the target tissue was at body temperature (e.g. not pre-warmed by dose DOE1).

In some embodiments, EDC 300 is configured as a monitoring device, such as when one or more sensors of system 10 provide physiologic information of the patient and/or information related to the patient's environment. In some embodiments, EDC 300 configures (e.g. sets the energy delivery parameters of) dose DOE1 and/or dose DOE2 based on this information. For example, EDC 300 can be configured to provide dose DOE1 and/or dose DOE2 based on patient physiologic information selected from the group consisting of: cardiac cycle; heart rate; blood pressure; blood flow rate; respiration rate; brain activity; electrogram amplitude (e.g. as measured in unipolar and/or bipolar modes); tissue impedance; and combinations of these.

In some embodiments, EDC 300 is configured to measure (e.g. and then prognose and/or otherwise estimate) the efficacy of the creation of a chronically effective lesion (e.g. the 6 month and/or 12-month efficacy of a lesion created by applying energy to tissue via EDD 100). Such an efficacy measurement may be a function of one, two, or more parameters (“input data” herein), such as energy delivery time, energy delivery power (e.g. average power delivered and/or cumulative power delivered), and/or proximity to and/or contact force between the one or more energy delivery elements delivering the energy and the tissue receiving the energy. Algorithm 335 can be configured to calculate a value indicative of the efficacy (e.g. 6- or 12-month efficacy) of an “ablation index”, lesion quality index 326. Lesion quality index 326 can represent (e.g. quantitatively and/or qualitatively represent) the current status of a lesion being created (e.g. a value which changes as energy is being delivered), and/or index 326 can represent the status of an already created lesion (e.g. provided in a table or other form for a single lesion or set of previously created lesions). Algorithm 335 can apply and/or otherwise implement a transfer function to determine lesion quality index 326 based upon the input data.

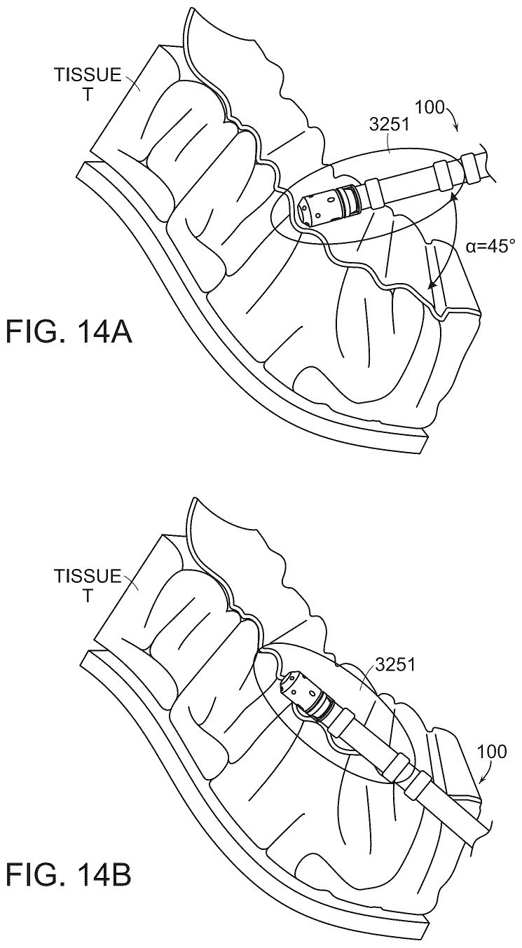

Algorithm 335 can be configured to determine (e.g. calculate) a geometric boundary defining a volume within which an electric field generated by EDD 100 is sufficient to irreversibly electroporate target tissue (referred to as a “Predicted PFA Zone” herein). In some embodiments, the Predicted PFA Zone can comprise an “isopotential boundary” within which any tissue would be irreversibly electroporated. A representation of the Predicted PFA Zone can be displayed to the user via a boundary overlay 3251 shown (also referred to as overlay 3251), such as an overlay displayed via GUI 325 relative to a visual representation of EDD 100. Boundary overlay 3251 can be of a similar arrangement to overlays described herein in reference to FIGS. 14-16 and otherwise herein. In some embodiments, overlay 3251 can convey to the user the approximate size and shape of an electric field created by an IEP pulse and locations in which that field intersects with target tissue. The Predicted PFA Zone determined by algorithm 335 can be based on one, two, or more parameters selected from the group consisting of: electrode source and/or return configuration parameters (e.g. between which electrodes 130 is the electric field generated); the configuration of EDD 100 (e.g. the spacing of electrodes 130 on EDD 100); the voltage of the energy delivered to generate the electric field; the parameters of one or more doses DOE delivered (e.g. within 30 seconds) and/or to be delivered; the parameters of one or more doses DOE previously delivered, such as doses delivered at least 30 seconds prior; and combinations of these. The Predicted PFA Zone can comprise a probabilistic metric, such as a metric based at least in part on the one, two, three, or more doses DOE that are delivered to a particular location over time (e.g. how much energy has been delivered to a location throughout a procedure). In some embodiments, boundary overlay 3251 comprises multiple regions, such as multiple regions representing various Predicted PFA Zones, such as predicted zones based on various treatment scenarios, such as scenarios wherein multiple doses DOE1 and DOE2 are delivered to tissue (e.g. versus a scenario where only a single dose DOE2 is delivered). In some embodiments, algorithm 335 is configured to perform one or more simulations (e.g. electric field simulations) to determine the Predicted PFA Zone. In some embodiments, the Predicted PFA Zone comprises a volume within which there is a high probability tissue will be effectively treated (e.g. irreversibly electroporated), such as a probability that at least 50% of tissue within the zone will be effectively treated, such as at least 60%, at least 70%, at least 80%, and/or at least 90% of tissue within the zone will be effectively treated. In some embodiments, a property (e.g. a graphical property) of overlay 3251 represents the probability of efficacious treatment, for example the opacity of overlay 3251 can correlate to the probability, such as is described in reference to FIGS. 15A-C herein.

In some embodiments, algorithm 335 can be configured to calculate a therapy effectiveness score throughout a volume of space (e.g. a volume of tissue such as the Predicted PFA zone described herein). This therapy effectiveness score, termed a “volume-distributed score” or “VDS” herein, can be calculated for a volume surrounding an energy-delivering portion of one or more devices. The calculated VDS can represent an estimation of therapy effectiveness for a future delivery of energy (a “Predicted VDS” herein that is based on one or more proposed energy delivery parameters) and/or a calculation of therapy effectiveness of an energy delivery already performed by system 10 (a “Actual VDS” herein). The volume of space represented by the VDS can extend far enough to encompass regions that are highly probable, marginally probably, and/or minimally probable of receiving a therapeutically effective delivery of energy (e.g. delivery of irreversible electroporation energy). Successful delivery of energy can comprise delivery of multiple energy deliveries (e.g. multiple IEP pulses delivered simultaneously and/or sequentially), whose combination achieves a desired result (e.g. a desired irreversible electroporation of target tissue).

A desired clinical result can include one result of a set of desired (e.g. acceptable) results, such as a result in which target tissue is unaltered, transiently altered, and/or permanently altered. Such an alteration can comprise: an electrical alteration of tissue (e.g. where cells of the tissue are stunned, or killed); a biophysical alteration of tissue (e.g. when the tissue is thickened, tissue is thinned, and/or edema results); a biochemical alteration of tissue; a material alteration of tissue (e.g. tissue is stiffened, softened, made porous, and/or made brittle), and combinations of these.

A VDS can be based on a single metric, such as electrical field strength, or it can be based on more than one metric, such as two or more metrics selected from the group consisting of: electric field strength; area of contact; angle of contact; force of contact; temperature; impedance; one or more tissue properties; blood properties; and combinations of these. In some embodiments, a VDS score can ignore the quantitative impact of various tissue and/or tissue variations (e.g. variations between blood and myocardial tissue) on the electric field created by an IEP pulse (e.g. when the impact of the tissue properties on the electric field is negligible).

For an application of therapy (e.g. for each past and/or potential future energy delivery), algorithm 335 can calculate a VDS (e.g. an Actual VDS or Predicted VDS, respectively), such as by taking into account one or more of: the energy delivery configuration (e.g. energy delivery element 130 configuration); EDC 300 configuration (e.g. the configuration of the energy waveform provided and/or other energy delivery parameter); and/or the energy delivery environment (e.g. blood geometry; target tissue geometry; and/or properties relative to delivery device). The VDS can be configured to include the results of a field model used by algorithm 335, such as a field model that incorporates the EDC 300 configuration and/or the energy delivery environment. Algorithm 335 can use a field model that is based on: analytical calculations and/or equations; discrete and/or numerical methods (e.g. randomized point sources with modeled boundary conditions); and/or a look-up table and/or estimation and/or optimization algorithm with data fitting based on a priori data (e.g. simulation, database, or training dataset). An optimization algorithm included in algorithm 335 can include statistical models, pattern matching, machine learning, artificial intelligence, and/or other data-driven techniques (e.g. techniques based on historical data).

System 10 can store both the VDS as well as all the parameters that contributed to the calculation of the VDS, such that if one or more of the parameters is updated with additional information (e.g. more accurate information), one or more VDS values can be recalculated and/or otherwise updated. For example, system 10 can store field strength, the locations of energy delivery elements 130, tissue location, tissue orientation, tissue properties, and VDS values for all delivered and/or proposed energy delivery applications. When the tissue location, tissue orientation, and/or other tissue properties are updated, the associated VDS values can be recalculated in any areas where the updated parameters have an effect. The VDS values can be stored in a spatially distributed data structure, such as a rectangular voxel grid. The data structure can be used to keep track of VDS values in the same and/or overlapping spatial location from multiple energy delivery applications. Algorithm 335 can assess the combination of two or more VDS values to produce a “composite VDS”. The composite VDS can result in a determination (e.g. a prognosis or other estimation) of therapeutic effectiveness, while the constituent VDS values can each result in individual determinations of a lack of effectiveness.

Once a set of VDS values is available, the values can be displayed through a user interface (e.g. GUI 325), such as to provide the values to an operator of system 10 (e.g. a clinician of the patient). The spatial distribution of VDS values can be shown (e.g. differentiated) in the interface (e.g. a 3D interface), such as by using color, opacity, intensity, and/or another variable graphical parameter. In some embodiments, system 10 is configured to display VDS values in a visual form similar to a bead of material exiting a caulking gun, such as when the bead has an elongate structure of varying 3D dimension. System 10 can limit the display to include only VDS values that exceed a threshold, such as a threshold that can be set or changed by the user (e.g. dynamically sweep the threshold value), such as to visually assess the variability of scoring differences in different areas. This threshold-based configuration can be used to assess the presence of “vulnerable” areas that may require further treatment, such as is described herebelow.

In some embodiments, system 10 can be configured to display only the portion of the VDS that will be and/or has been delivered into target tissue (e.g. one or more portions of heart wall tissue). For example, when delivering energy from one or more locations within a cavity (e.g. a chamber of the heart) and into target tissue (e.g. one or more portions of heart wall tissue), system 10 can display the VDS portion that is “beyond” the endocardial surface reconstruction provided by system 10 (e.g. outside the surface included in the anatomical model created by system 10 as described herein). This display method does not require data representing the thickness of the associated tissue (e.g. the thickness of the cardiac wall). However, if the thickness of any portion of the wall surrounding the chamber is known, system 10 can be configured to display only the portion of the VDS that is delivered into the wall tissue.

System 10 can be configured to display VDS values as a projection onto an anatomic model produced by system 10 (e.g. an anatomic model produced via ultrasound and/or other sensors as described herein). System 10 can calculate each VDS in a normal direction outward from the anatomic model. The VDS value along this direction can be stored in the form of a data series that is a function of distance and/or depth. This data series can optionally be displayed in the form of a graph and/or plot. A VDS threshold can be set to delineate a therapy quality limit such that the “effective” distance into the target tissue can be quantified. The effective distance into the target tissue can be displayed (e.g. quantified and/or otherwise differentiated) using an array of colors, opacity, intensity, and/or other variable graphical parameter. The threshold can be dynamically adjusted to visualize the distribution of VDS in different areas. This display method can be used to assess areas of vulnerability that may require further action (e.g. further energy delivery). System 10 can calculate the cumulative VDS, maximum VDS, combinatorial VDS, or other mathematical operation of various VDS data.

Prior to application of therapy (e.g. delivery of energy), an estimated VDS can be dynamically calculated by system 10 based on present energy delivery configuration, energy delivery environment parameters, and/or contact force between EDD 100 and target tissue. A 3D display of Predicted VDS values can be dynamically presented to indicate to the user the anticipated result of therapy application at that moment. The display can use augmented visuals (variation in color, transparency, pattern, labels, and/or other graphical parameters) to distinguish delivered therapy versus potential future therapy. The display can be based on the presence of contact force and/or the degree of contact force. This display of Predicted VDS values can be used by the user to make necessary adjustments to the delivery configuration (e.g. position, angle, electrode selection, waveform, and/or other generator configuration), such as to optimize the desired energy delivery. Numeric thresholds can be used to modify the displayed Predicted VDS values. The display of the Predicted VDS values can also include unique visual features to highlight the location and/or the effect of additionally applied therapy based on its interaction with previously delivered therapy. For example, the Predicted VDS display can include areas of overlap with previously delivered therapy (e.g. an energy delivery for which an Actual VDS has been calculated) that would add, advance, and/or otherwise improve the portion of the Predicted VDS that overlaps the actual VDS. The overlapping portion can be displayed differently from non-overlapping portions. This difference can be displayed via variations in color, opacity, intensity, and/or other graphical parameter.

Algorithm 335 can comprise a “vulnerability algorithm” that processes a set of Actual VDS values of a region to qualitatively label and/or quantitatively display areas in which the delivered therapy (e.g. delivered energy) may be below a desired effectiveness threshold. The vulnerability algorithm can process a set of VDS values to identify data features that are deterministically and/or probabilistically ineffective. The vulnerability algorithm can be configured to search for spatial gaps, edges, channels, and/or spatial heterogeneity in the VDS values. The vulnerability algorithm can use the intra-tissue VDS distance and project to a two-dimensional plane, such as to advantageously process the spatial information with less dimensional complexity such as to improve speed, accuracy, and/or other performance parameter. The vulnerability algorithm can utilize image processing and/or pattern matching techniques to assess vulnerability. The vulnerability algorithm can include statistical models, pattern matching, machine learning, artificial intelligence and/or other data driven techniques, such as when based on and/or when configured to analyze historical data.

Referring now to FIG. 2, a flow chart of an embodiment of a method of delivering energy to tissue is illustrated, consistent with the present inventive concepts. Method 2000 is described using system 10 and its components, as described herein.

In STEP 2010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 2020, one or more energy delivery elements 130 of EDD 100 are moved proximate a tissue site for treatment, “target tissue” herein.

In STEP 2030, a first dose of energy, dose DOE1 as described herein, is provided by EDC 300 to EDD 100 and delivered to the target tissue by the one or more energy delivery elements 130.

In STEP 2040, a second dose of energy, dose DOE2 as described herein, is provided by EDC 300 to EDD 100 and delivered to the target tissue by the one or more energy delivery elements 130 (e.g. the same and/or different energy delivery elements 130 that delivered dose DOE1 in STEP 2030).

In STEP 2050, a check of procedure completeness is performed. If the procedure is not complete, such as when additional tissue (e.g. additional target tissue) is to be treated, the method returns to STEP 2020. If the procedure is complete, the procedure is ended in STEP 2070.

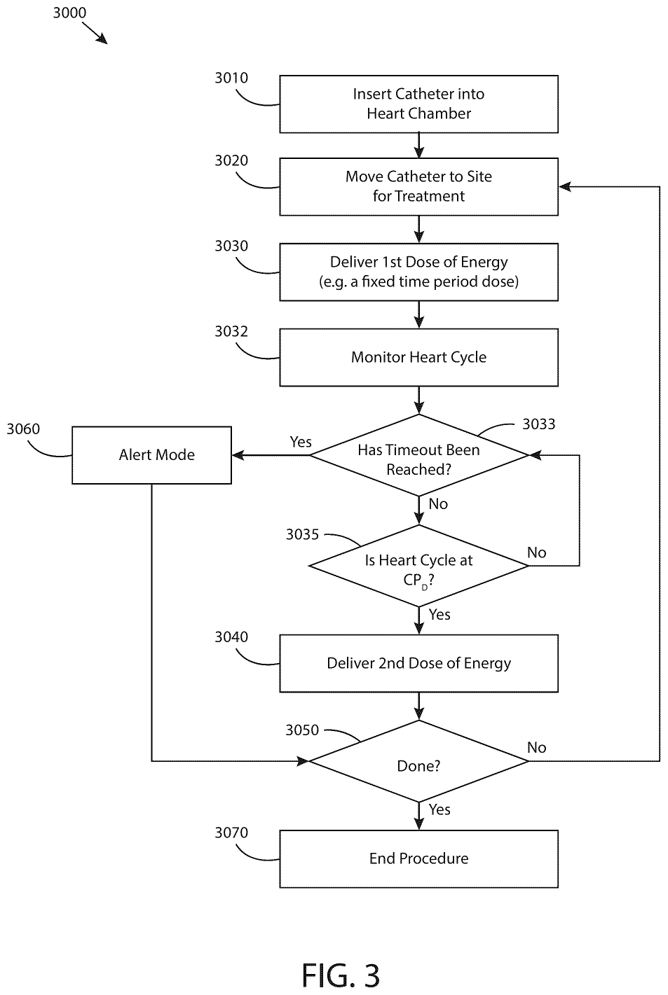

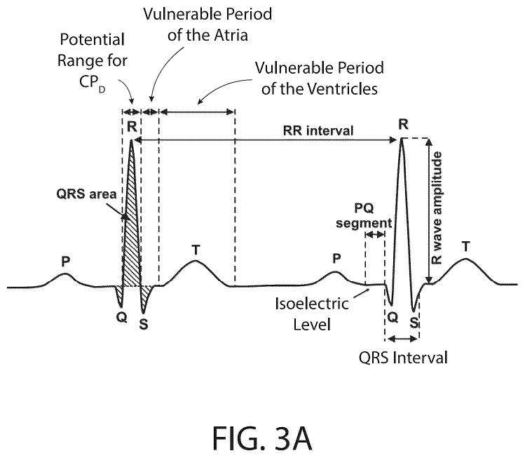

Referring now to FIG. 3, a flow chart of an embodiment of a method of delivering energy to tissue is illustrated, consistent with the present inventive concepts. Method 3000 is described using system 10 and its components, as described herein.

In STEP 3010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 3020, one or more energy delivery elements 130 of EDD 100 are moved proximate target tissue.

In STEP 3030, a first dose of energy, dose DOE1 as described herein, is provided by EDC 300 to EDD 100 and delivered to target tissue by the one or more energy delivery elements 130. In some embodiments, the dose DOE1 comprises delivery of energy (e.g. RF energy) for a fixed time period. In some embodiments, STEP 3030 is triggered (e.g. started) based on the patient's heart cycle (e.g. such that STEP 3035 can be performed at a subsequent point in the patient's heart cycle).

In STEP 3032, the heart cycle of the patient is monitored, such as by EDC 300 or other component of system 10.

In STEP 3033, a check of reaching a “timeout” is performed, such as a timeout period comprising the time since the dose DOE1 delivery finished. If the timeout period has been reached, STEP 3060 is performed in which system 10 enters an alert mode, and method 3000 continues to STEP 3050 described herebelow. If the timeout period has not been reached, STEP 3035 is performed.

In STEP 3035, a check to determine if the patient's heart cycle is at a desired cycle point, cycle point CP_{D}. If the patient's heart cycle is not at point CP_{D}, method 3000 returns to STEP 3033. If the patient's heart cycle is at point CP_{D}, STEP 3040 is performed.

In STEP 3040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 3030).

In some embodiments, cycle point CP_{D} is selected in STEP 3035 such that in STEP 3040 dose DOE2 is delivered at a time between 50 msec and 200 msec after the R wave occurs, reference FIG. 3A.

In STEP 3050, a check of procedure completeness is performed. If the procedure is not complete, such as when additional tissue (e.g. additional target tissue) is to be treated, the method returns to STEP 3020. If the procedure is complete, the procedure is ended in STEP 3070.

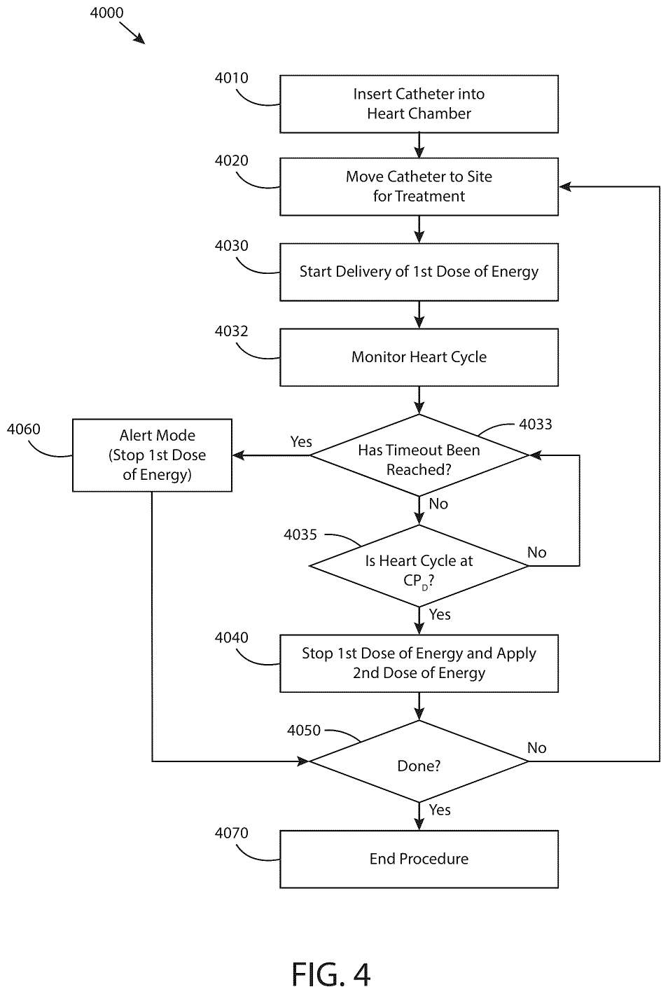

Referring now to FIG. 4, a flow chart of an embodiment of a method of delivering energy to tissue is illustrated, consistent with the present inventive concepts. Method 4000 is described using system 10 and its components, as described herein.

In STEP 4010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 4020, one or more energy delivery elements 130 of EDD 100 are moved proximate target tissue.

In STEP 4030, delivery of a first dose of energy, dose DOE1 as described herein, is initiated. Energy is provided by EDC 300 to EDD 100 and delivered to target tissue by the one or more energy delivery elements 130. In some embodiments, STEP 4030 is triggered (e.g. started) based on the patient's heart cycle (e.g. such that STEP 4035 can be performed at a subsequent point in the patient's heart cycle).

In STEP 4032, the heart cycle of the patient is monitored, such as by EDC 300 or other component of system 10.

In STEP 4033, a check of reaching a “timeout” is performed, such as a timeout period comprising the time since the dose DOE1 delivery finished. If the timeout period has been reached, STEP 4060 is performed in which system 10 enters an alert mode, delivery of dose DOE1 is stopped, and method 4000 continues to STEP 4050 described herebelow. If the timeout period has not been reached, STEP 4035 is performed.

In STEP 4035, a check to determine if the patient's heart cycle is at a desired cycle point, cycle point CP_{D}, is performed. If the patient's heart cycle is not at point CP_{D}, method 4000 returns to STEP 4033. If the patient's heart cycle is at point CP_{D}, STEP 4040 is performed. Monitoring of the patient's heart cycle can be performed by one or more components of system 10, during STEP 4035 and/or during other steps of method 4000.

In STEP 4040, delivery of dose DOE1 is stopped, and a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 4030).

In STEP 4050, a check of procedure completeness is performed. If the procedure is not complete, such as when additional tissue (e.g. additional target tissue) is to be treated, the method returns to STEP 4020. If the procedure is complete, the procedure is ended in STEP 4070.

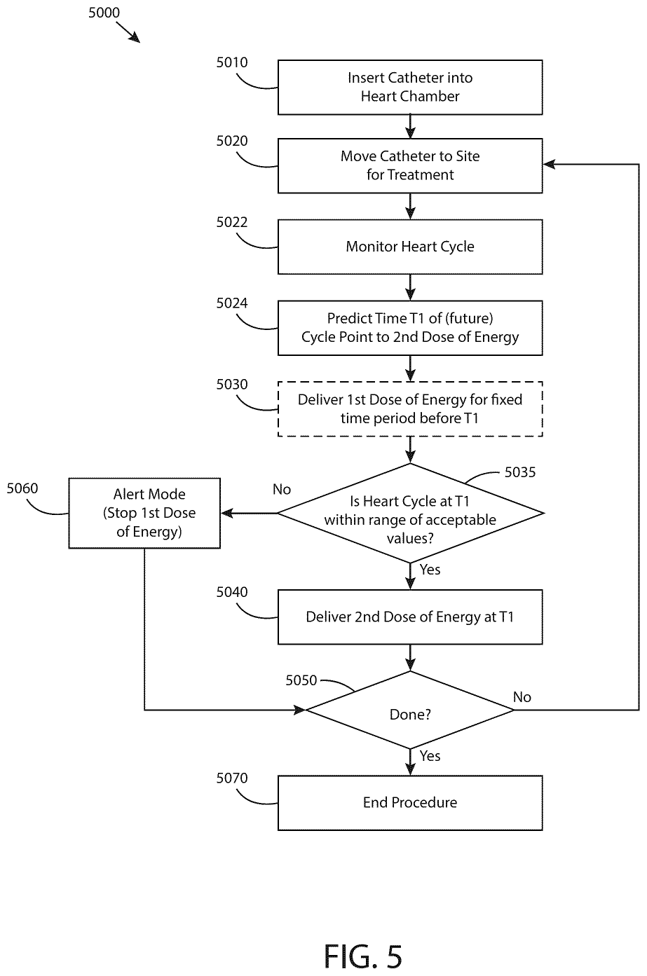

Referring now to FIG. 5, a flow chart of an embodiment of a method of delivering energy to tissue is illustrated, consistent with the present inventive concepts. Method 5000 is described using system 10 and its components, as described herein.

In STEP 5010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 5020, one or more energy delivery elements 130 of EDD 100 are moved proximate target tissue.

In STEP 5022, the heart cycle of the patient is monitored, such as by EDC 300 or other component of system 10.

In STEP 5024, a time T1 of a future desired heart cycle point CP_{D} is predicted by system 10 (e.g. via algorithm 335).

In STEP 5030, an optional step of delivering a first dose of energy, dose DOE1 as described herein, can be provided by EDC 300 to EDD 100 and delivered to target tissue by the one or more energy delivery elements 130. In some embodiments, the dose DOE1 comprises delivery of energy (e.g. RF energy) for a fixed time period (e.g. a fixed time period that ends prior to time T1).

In STEP 5035, a check is performed (e.g. at time T1 or just prior) to determine if the patient's heart cycle at time T1 is at a desired cycle point, cycle point CP_{D}. If the patient's heart cycle is at point CP_{D}, STEP 5040 is performed. If the patient's heart cycle is not at point CP_{D}, STEP 5060 is performed in which system 10 enters an alert mode, delivery of dose DOE1 is stopped (if being delivered via optional step S030), and method 5000 continues to STEP 5050 described herebelow.

In STEP 5040, a dose of energy (e.g. a first or second dose of energy), dose DOE2 as described herein, is provided by EDC 300 to EDD 100 and delivered to target tissue by the one or more energy delivery elements 130 (e.g. the same and/or different energy delivery elements 130 that may have delivered dose DOE1 in optional STEP 5030).

In STEP 5050, a check of procedure completeness is performed. If the procedure is not complete, such as when additional tissue (e.g. additional target tissue) is to be treated, the method returns to STEP 5020. If the procedure is complete, the procedure is ended in STEP 5070.

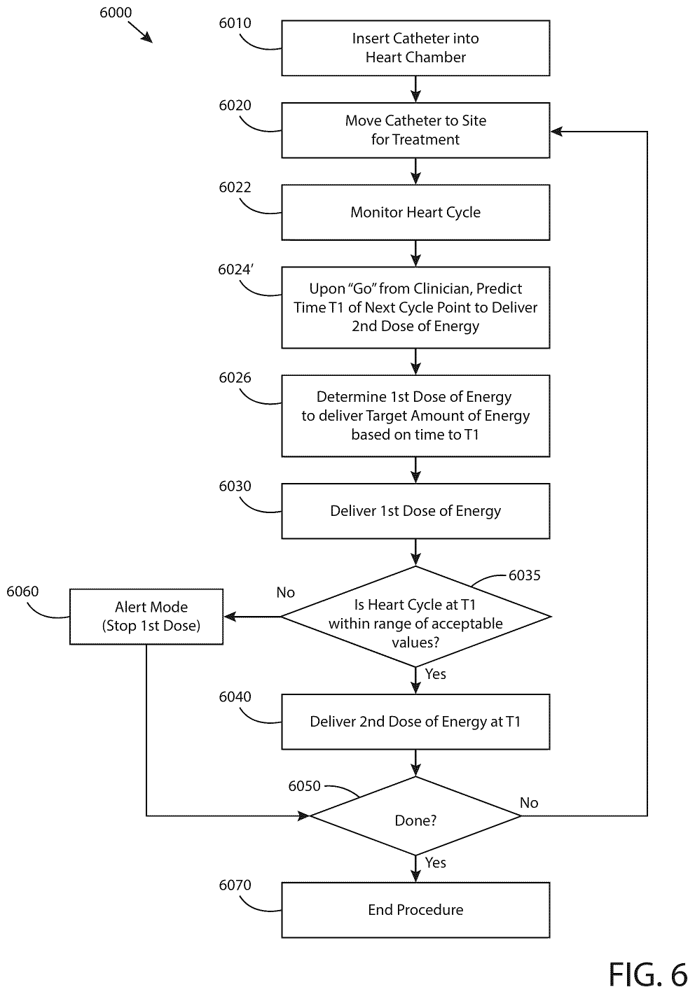

Referring now to FIG. 6, a flow chart of an embodiment of a method of delivering energy to tissue is illustrated, consistent with the present inventive concepts. Method 6000 is described using system 10 and its components, as described herein.

In STEP 6010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 6020, one or more energy delivery elements 130 of EDD 100 are moved proximate target tissue.

In STEP 6022, the heart cycle of the patient is monitored, such as by EDC 300 or other component of system 10.

In STEP 6024′, after a “go” signal (e.g. an initiation request) is received from an operator of system 10 (e.g. the patient's clinician as provided via user interface 320 of EDC 300), a time T1 of a future desired heart cycle point CP_{D} is predicted by system 10 (e.g. via algorithm 335).

In STEP 6026, a first dose of energy, dose DOE1 is determined to achieve a target amount of energy (e.g. a target amount of Joules) that is to be delivered by time T1 (e.g. delivered continuously to time T1 and/or in intermittent pulses up till time T1). This target amount of energy can be determined by system 10 and/or a clinician of the patient.

In STEP 6030, dose DOE1 as described herein and defined in STEP 6026, is provided by EDC 300 to EDD 100 and delivered to target tissue by the one or more energy delivery elements 130.

In STEP 6035, a check is performed (e.g. at time T1 or just prior) to determine if the patient's heart cycle at time T1 is at a desired cycle point, cycle point CP_{D}. If the patient's heart cycle is at point CP_{D}, STEP 6040 is performed. If the patient's heart cycle is not at point CP_{D}, STEP 6060 is performed in which system 10 enters an alert mode, delivery of dose DOE1 is stopped, and method 6000 continues to STEP 6050 described herebelow.

In STEP 6040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 6030).

In STEP 6050, a check of procedure completeness is performed. If the procedure is not complete, such as when additional tissue (e.g. additional target tissue) is to be treated, the method returns to STEP 6020. If the procedure is complete, the procedure is ended in STEP 6070.

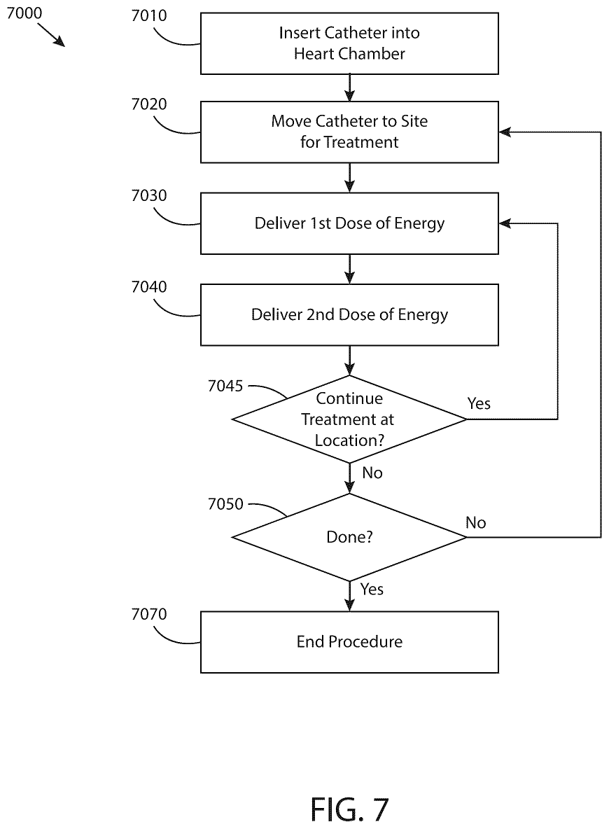

Referring now to FIG. 7, a flow chart of an embodiment of a method of delivering energy to tissue is illustrated, consistent with the present inventive concepts. Method 7000 is described using system 10 and its components, as described herein.

In STEP 7010, the distal portion of EDD 100 is inserted into a heart chamber of a patient. In some embodiments, the distal portion of catheter 200 is also inserted into a heart chamber of the patient, such as to provide biopotential measurement, anatomical visualization, and/or other cardiac mapping functions.

In STEP 7020, one or more energy delivery elements 130 of EDD 100 are moved proximate target tissue.

In STEP 7030, dose DOE1 as described herein, is provided by EDC 300 to EDD 100 and delivered to target tissue by the one or more energy delivery elements 130.

In STEP 7040, a second dose of energy, dose DOE2 as described herein, is provided by EDC 300 to EDD 100 and delivered to target tissue by the one or more energy delivery elements 130 (e.g. the same and/or different energy delivery elements 130 that delivered dose DOE1 in STEP 7030).

In STEP 7045, a check of whether additional energy should be delivered to the current target tissue is performed. If additional energy delivery is to be delivered (e.g. as determined manually by an operator of system 10 and/or automatically by algorithm 335, such as when algorithm 335 comprises an AI algorithm), Method 7000 returns to repeat STEP 7030. Otherwise, STEP 7050 is performed.

In STEP 7050, a check of procedure completeness is performed. If the procedure is not complete, such as when additional tissue (e.g. additional target tissue) is to be treated, the method returns to STEP 7020. If the procedure is complete, the procedure is ended in STEP 7070.

In Method 7000 of FIG. 7, the delivery of energy in STEPS 7030 and 7040 can comprise delivering of doses DOE1 and DOE2 in an interleaving arrangement (e.g. at least a portion of dose DOE1 is delivered during STEP 7040 and/or at least a portion of dose DOE2 is delivered during STEP 7030). For example, a portion of dose DOE1 can be delivered during a portion of dose DOE2 or vice versa. In some embodiments, dose DOE1 comprises energy (e.g. RF energy) delivered in pulsed arrangement, such as when dose DOE1 comprises one or more periods of “on time” (DOE1TP_{ON}), separated by “off time” periods (DOE1TP_{OFF}), where energy is delivered during DOE1TP_{ON} periods, and no energy is delivered during DOE1TP_{OFF} periods (e.g. a pulse-width modulation arrangement). In these embodiments, dose DOE2 can be delivered during DOE1TP_{OFF} periods of dose DOE1. In some embodiments, dose DOE2 comprises energy (e.g. an IEP dose) delivered in pulsed arrangement, such as when dose DOE2 comprises one or more periods of “on time” (DOE2TP_{ON}) separated by “off time” periods (DOE2TP_{OFF}), where energy is delivered during DOE2TP_{ON} periods, and no energy is delivered during DOE2TP_{OFF} periods (e.g. a pulse-width modulation arrangement). In these embodiments, dose DOE1 can be delivered during DOE2TP_{OFF} periods of dose DOE2. In some embodiments, doses DOE1 and DOE2 can each comprise energy delivered in a pulsed arrangement. In these embodiments, energy that is delivered during doses DOE1 and DOE2 can be delivered in an alternating arrangement, such as when energy delivery of dose DOE1 is delivered during a DOE2_{OFF} period of dose DOE2, and energy delivery of dose DOE2 is delivered during a DOE1_{OFF} period of dose DOE1.

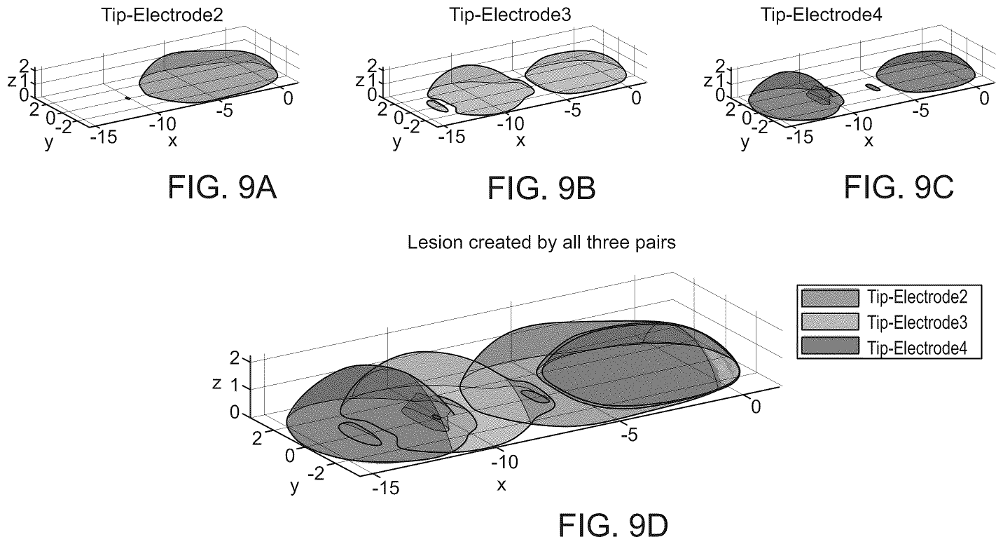

Referring now to FIG. 8, a side view of an embodiment of a distal portion of an energy delivery device is illustrated, consistent with the present inventive concepts. FIGS. 8A and 8B are two graphs of lesion depth versus electrode-pair during an electroporation study with the device of FIG. 8, consistent with the present inventive concepts. Referring additionally to FIGS. 9A-D, four graphs representing lesion volumes versus electrode-pair during the electroporation study with the device of FIG. 8 are illustrated, also consistent with the present inventive concepts. Applicant has conducted in-silico studies where an EDD 100, such as EDD 100 shown in FIG. 8, delivers a dose of energy comprising an IEP, as defined herein, to tissue (an IEP dose provided by EDC 300 as described herein in reference to FIG. 1). EDD 100 of FIG. 8 comprises four electrodes 130 (e.g. four electrode-based energy delivery elements 130) positioned in distal portion 102. EDD 100 comprises an energy delivery element, electrode 130a, positioned on the distal end of shaft 110, and three energy delivery elements (e.g. electrodes 130b-d) positioned more proximally on shaft 110, in a sequential, linear arrangement. Electrode 130b is positioned most proximate to tip electrode 130a, electrode 130c is positioned distal to electrode 130b. and electrode 130d is positioned distal to electrode 130c, all as shown in FIG. 8. Results of the in-silico studies indicated the shape and size of a lesion in target tissue can be controlled by selecting a particular pair of electrodes 130, and having that pair deliver an IEP. FIGS. 8A-B illustrate testing from 3 pairs of electrodes 130, where pair “1-2” was electrode 130a and 130b, pair “1-3” was electrode 130a and 130c, and pair “1-4” was electrode 130a and 130d. In the simulation, the distal portion of EDD 100 was positioned approximately orthogonal (90°) to the tissue surface to receive the energy, electrode 130a had a length of 3.46 mm, the amplitude of the electric field was held constant. Tests were repeated for each of the 3 pairs at different voltages, 500V, 1000V, 1500V, and 2000V, as marked on the figure. FIG. 8A is a graph of lesion depth for each electrode 130 pair, while FIG. 8B is a graph of lesion surface area for each electrode 130 pair. FIGS. 9A-C are graphs of the volume of lesions created using pair 1-2, 1-3, and 1-4, respectively, where the distal portion of EDD 100 was placed on the tissue (e.g. at an angle of approximately) 0°. FIG. 9D is a graph of the combined (superimposed) lesions of FIGS. 9A-C.

As shown by the studies conducted by applicant, system 10 can be configured to switch between pairs of electrodes 130 receiving and/or delivering an IEP (without modifying amplitude), to modify lesion depth and/or to create lesions with desired geometric volumes. For example, as shown in FIG. 8A, a lesion created with an IEP of 1000V creates a deeper lesion when delivered by pair 1-3 than when delivered by pair 1-2. As shown in FIGS. 9A-D, a longer lesion (about 1.5 cm as shown) can be created by delivering IEP doses between multiple pairs of electrodes (e.g. without having to reposition the distal portion of EDD 100).

Referring now to FIGS. 10A-B, two anatomical sectional views of the distal portion of an embodiment of an energy delivery device contacting a tissue surface at different angles of orientation are illustrated, consistent with the present inventive concepts. As described herein, system 10, via EDD 100, can be configured to deliver one or more electric pulses between two or more energy delivery elements, electrodes 130 shown (e.g. one or more electrodes 130 configured to source current, and one or more electrodes 130 configured to sink current). The parameters of each of the pulses (e.g. voltage, current, frequency, pulse width, and the like) can be selected to create high voltage electric fields within tissue proximate the two or more electrodes, such as to perform “pulsed field ablation” to ablate the desired target tissue. The energy is provided to EDD 100 by EDC 300, as described herein. The parameters of the electrical pulses can further be selected such that the resultant electric field causes reversible or irreversible electroporation of the target tissue. The spatial extent (e.g. complete volume) of the tissue ablated and the effectiveness of the pulsed field ablation depends on the strength of the electric field at the target location (e.g. the target location selected for ablation to provide a therapeutic benefit to the patient as described herein). The strength of the electric field is related to the distance from the electrodes (e.g. electrodes 130 mounted to shaft 110 of EDD 100 as shown), where the field strength decreases exponentially with increasing distance from the electrodes. System 10 is configured to provide field strength at a sufficient level for all target tissue (e.g. all intended widths, lengths, and depths of target tissue) to be ablated.

System 10 (e.g. algorithm 335 described herein) can be configured to determine a lesion size parameter (e.g. a length, a width, a depth, and/or a volume of the lesion), L_{P}, where L_{P} is a function of: peak voltage of the pulsed field ablation (PFA), pulse V_{PEAK}; an angle of orientation a; and/or one or more electrophysical parameters EP_{P}. Angle α is the angle between the axis of distal portion 102 of EDD 100 (e.g. including an electrode and/or other energy delivery component), and the plane of the tissue surface proximate distal portion 102 of EDD 100. In FIG. 10A, angle α is 90° (i.e. distal portion 102 of EDD 100 is orthogonal to the neighboring tissue surface), and in FIG. 10B, angle α is 0° (i.e. distal portion 102 of EDD 100 is parallel and in contact with the neighboring tissue surface). Parameter EP_{P} can include one, two, or more of: contact force, pulse amplitude, pulse duration, number of pulses, number of electrodes sourcing and/or sinking current; tissue temperature, and/or tissue impedance.

In some embodiments, EDD 100 comprises a sensor or other component configured to determine orientation angle α, such as to be used in a calculation to determine lesion size as described hereabove. For example, one or more sensors (e.g. sensing elements 158 as described herein) of force maintenance assembly 150 can be configured to provide a signal from which angle α can be determined (e.g. by algorithm 335), such as when force maintenance assembly 150 comprises optical fibers, magnetic sensors, impedance measurement sensors, and/or other sensing elements configured to provide a signal related to angle α. Alternatively or additionally, angle α can be determined via signals provided by mapping and/or navigation sensors of system 10, such as when algorithm 335 performs impedance and/or magnetic based localization to determine angle α. Alternatively or additionally, system 10 can comprise an imaging device, not shown, but such as an imaging device selected from the group consisting of: intracardiac ultrasound image; x-ray; fluoroscope; magnetic resonance imager; computed tomography imager; visible light camera; infrared camera; and combinations of these. Algorithm 335 can utilize information provided by the imaging device to determine angle α.

In some embodiments, an electrical pulse is applied between tip electrode 130a and one or more neighboring electrodes 130 (e.g. electrodes 130b, 130c, and/or 130d shown). As it relates solely to angle α, the electric field strength in the tissue is at a minimum when angle α is at 90° as shown in FIG. 10A, and the field strength increases as angle α decreases from 90°, eventually reaching maximum when angle α is at 0° as shown in FIG. 10B. In other words, when one or more PFA pulses are delivered by EDD 100 to target tissue, the spatial extent of the resultant lesion (e.g. the depth of the resultant lesion) increases as the electrodes 130 associated with each PFA pulse (e.g. the electrodes sourcing and/or sinking current) are moved closer to the tissue surface.

Referring now to FIGS. 11A-B, two user's views of an embodiment of a graphical user interface displaying information related to different angles of orientation of an energy delivery device are illustrated, consistent with the present inventive concepts. System 10, via user interface 320 of EDC 300, can include a graphical user interface, GUI 325 shown. GUI 325 can be configured to provide information related to orientation angle α described herein, such as is provided by orientation angle representation 3250 shown. GUI 325 can display current (e.g. real time) position information related to EDD 100 (e.g. related to distal portion 102 of EDD 100). GUI 325 can include: catheter representation 3210 representing the position of distal portion 102; and/or tissue representation 3220 representing the position of a tissue surface to be ablated (e.g. the tissue surface proximate distal portion 102); each as shown.

GUI 325 can further comprise indicator graph 3230, which can provide field strength feedback to the operator, such as a graphical representation of an estimate of the field strength in the tissue proximate electrodes 130. The indicator graph 3230 can indicate the field strength of energy (e.g. PFA pulses currently being delivered, or the field strength that would be present once an operator initiates energy delivery). Information provided by indicator graph 3230, and other information provided by GUI 325, can be used by an operator (e.g. in an iterative and/or other adjustable arrangement), to create a lesion of a desired size (e.g. a desired length, width, and/or depth dimension). Indicator graph 3230 can include first marker 3231 and second marker 3232, each as shown. The relative position between the markers 3231 and 3232 can correlate to the field strength in tissue, such that marker 3231 approaches marker 3232 as the field strength increases (e.g. as shown in the transition from the orthogonal catheter orientation depicted in catheter representation 3210 of FIG. 11A transitioning to the catheter orientation depicted in catheter representation 3210 of FIG. 11B in which angle α is) 5°. It should be understood that system 10 can provide various other forms of visual feedback to an operator regarding field strength and/or other ablation parameter of a current (e.g. real time) or future (to be delivered based on current conditions) pulsed field ablation energy delivery.

In some embodiments, GUI 325 can be configured to provide information related to contact force, such as can be visually provided by contact force representation 3260 shown, which includes force indicator 3261 representing the current contact force being applied (shown at the same level in each of FIGS. 11A and 11B). Contact force representation 3260 can further include threshold indicator 3262, which can indicate a required or recommended limit on contact force to be applied (e.g. an amount less than the maximum contact force available). The provided contact force information can provide an absolute measurement of force (e.g. a measurement expressed in grams or other metric indicating contact force), and/or a relative measurement (e.g. a percentage of a maximum amount of contact force). In some embodiments, GUI 325 is further configured to change the distance between first marker 3231 and second marker 3232 as contact force changes, for example the distance can decrease (e.g. indicating an increase in electric field in tissue) when contact force increases. In some embodiments, the distance between the two markers is based on both angle α and contact force, where an operator can change either or both to change the electric field strength in the tissue. In some embodiments, GUI 325 changes the distance between the two markers based on all or some of: angle α; contact force; and/or one or more electrophysical parameters EP_{P}.

Referring now to FIG. 12, a perspective view of the distal portion of an embodiment of an energy delivery device including multiple ports for delivering irrigation fluid is illustrated, consistent with the present inventive concepts. EDD 100 of FIG. 12 includes electrode 130a shown on distal portion 102. The distal portion 102 of EDD 100 can include one or more ports, ports 1305 (six shown) for delivering irrigation fluid 70, such as one or more similar or dissimilar irrigation fluids 70 provided by fluid delivery module 370 of EDC 300 as described herein. Two or more ports 1305 can be spatially distributed in a desired pattern, such as a pattern covering a portion or the majority of distal portion 102 including electrodes 130. Two or more ports 1305 can be connected to independent lumens for delivery of different irrigation fluids 70 (e.g. different fluids 70a, 70b, and the like, such as fluids of dissimilar conductivity as described herein). Alternatively or additionally, two or more ports 1305 can be connected to a common lumen, such as for delivery of the same irrigation fluid 70 (e.g. from a single source of irrigation fluid 70).

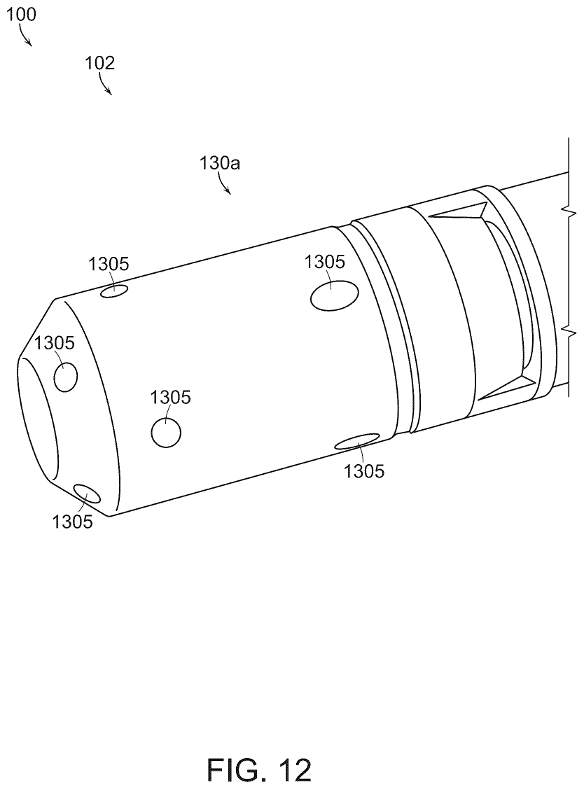

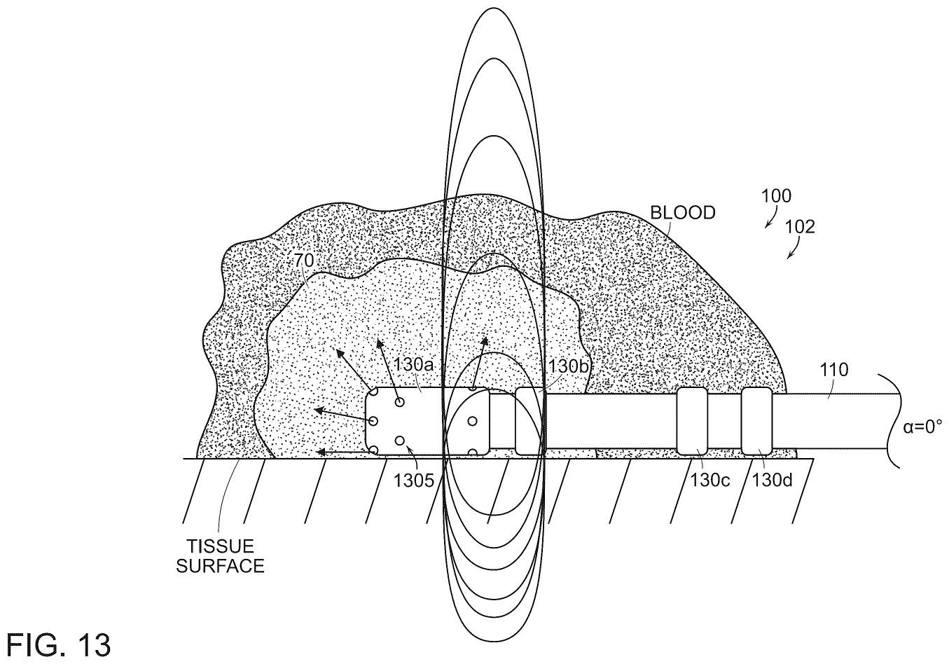

Referring additionally to FIG. 13, a side sectional anatomical view of the distal portion of an embodiment of an energy delivery device in contact with a tissue surface and delivering irrigation fluid is illustrated, consistent with the present inventive concepts. Distal portion 102 of EDD 100 is shown with an orientation angle α equal to 0°, such that each of electrodes 130a 130b, 130c and 130d are in contact with the tissue surface to be ablated. Irrigation fluid 70 is being delivered by ports 1305 (eight shown), and the delivered fluid 70 is surrounding electrodes 130a and 130b as shown (e.g. preventing blood, a relatively conductive substance, from surrounding those electrodes).

EDD 100 can be configured to deliver PFA pulses that create an electromagnetic field to ablate target tissue (e.g. to cause reversible or irreversible electroporation of tissue as described herein). One or more irrigation fluids 70 can be delivered, via ports 1305, to influence the pulsed electric field. For example, a delivered electrical field will “bunch” when passing through conductive media, and it will “spread” when passing through more resistive media, due to the electric current following the path of least resistance (i.e. the path of highest conductivity). System 10 can be configured to deliver one or more irrigation fluids 70 of known conductance to the area surrounding electrodes 130, and actively “steer” the delivered current and therefore the produced electric field.

In some embodiments, distal portion 102 of EDD 100 comprises at least six ports 1305 (e.g. twelve ports 1305), such that at least two ports 1305 (e.g. four ports 1305) are facing forward (e.g. facing distally from the distal end of shaft 110), at least two ports 1305 (e.g. four ports 1305) are located at the distal end of tip electrode 130a, and at least two ports 1305 (e.g. four ports 1305) are located at the proximal end of tip electrode 130. System 10 can include an irrigation fluid 70 having a different conductivity (e.g. lower conductivity) than the conductivity of blood. Prior to applying the pulsed electric field energy, EDD 100 can be oriented such that one or more ports 1305 are blocked via contact with tissue (e.g. blocked by the tissue surface of the left atrium or other chamber of the heart). Delivery of an irrigation fluid 70 via the remaining ports 1305 would have a steering effect on the desired electric field. For example, delivery of an irrigation fluid 70 having a lower conductivity than that of blood would concentrate the current delivered by electrode 130 into the contacting tissue, increasing the electric field into the tissue (e.g. as the fluid surrounding the electrode 130 delivering current is enveloped in the relatively low conductivity irrigation fluid 70).

Prior to, and/or during PFA pulse delivery, the angle of orientation a can be monitored as described herein (e.g. via one or more sensors of system 10 and/or by a separate imaging device), such as to provide feedback information relative to the electric field to be delivered and/or being delivered, where system 10 (e.g. algorithm 335, such as when algorithm 335 comprises an AI algorithm) can account for the increase in field strength (e.g. steering of the field) due to the delivery of the low conductance irrigation fluid 70.

Referring now to FIGS. 14A and 14B, side views of an embodiment of an energy delivery device positioned proximate sectional portions of tissue are illustrated, consistent with the present inventive concepts. FIG. 14A illustrates EDD 100, shown with its distal end positioned proximate tissue T, with an angle of orientation a of approximately 45°. FIG. 14B illustrates EDD 100 positioned proximate tissue T with an angle of orientation a of approximately 0°. GUI 325 can be configured to display images similar to those illustrated in FIGS. 14A-B. GUI 325 can include overlay 3251. Overlay 3251 can be similar to boundary overlay 3251 described herein in reference to FIG. 1. Overlay 3251 is shown relative to EDD 100 and represents the Predicted PFA Zone within which tissue T would be effectively treated (e.g. irreversibly electroporated) when energy is delivered via EDD 100 (e.g. at the current settings of system 10, which can be modified to accordingly modify the prediction of the zone receiving efficacious treatment, as described herein).

As shown in FIGS. 14A-B, overlay 3251 can comprise a 2D representation of the Predicted PFA Zone, where this zone comprises a 3D volume surrounding EDD 100 (e.g. surrounding an energy-delivering portion of EDD 100). In some embodiments, a 3D representation of overlay 3251 is displayed via GUI 325, such as when GUI 325 displays a 3D representation of an anatomic model of the heart, including a representation of EDD 100 relative to the model. As shown in FIGS. 14A-B, the amount of tissue within the Predicted PFA Zone can vary based on the size of the zone and/or the orientation of EDD 100 relative to the tissue. In FIG. 14B, more tissue is within the zone than is present within the similar sized zone illustrated in FIG. 14A, due to the different orientation of EDD 100 between the two figures.

Referring additionally to FIGS. 15A-15C, additional side views of an embodiment of an energy delivery device positioned proximate sectional portions of tissue are illustrated, consistent with the present inventive concepts. In some embodiments, the opacity of overlay 3251 is configured to represent a probability of effective treatment within the treatment zone (e.g. the probability that any cell within the zone of tissue being treated would be irreversibly electroporated). For example, the higher the opacity of overlay 3251 that is presented to an operator by system 10, the greater the likelihood that tissue within the zone would be effectively treated by an energy delivery of the present inventive concepts. For example, overlay 3251 of FIG. 15B is more opaque (i.e. has higher opacity) than overlay 3251 of FIG. 15A, and overlay 3251 of FIG. 15C is more opaque than overlay 3251 of FIG. 15B, which collectively represent an increasing probability of efficacy of a treatment to be performed. Numerous graphical properties can be used to quantify or qualify efficacy and/or other predictions provided by system 10. In some embodiments, algorithm 335 adjusts the probability of effective treatment based on a parameter selected from the group consisting of: the number of doses DOE delivered to a volume; the duration of the doses DOE of energy delivered; other parameters of the doses DOE delivered, such as the number of pulses of energy delivered for each DOE; and combinations of these. In some embodiments, as EDD 100 is maneuvered proximate tissue, overlay 3251 updates continuously (e.g. in real and/or near real time, “real time” herein), allowing the user to see how much energy has been delivered to an area of tissue. In some embodiments, overlay 3251 is updated at least once every 30 seconds, such as at least once every 15 seconds, 10 seconds, 5 seconds, 2 seconds, or 1 second. For example, as more energy is delivered to an area (e.g. via sequentially delivered doses DOE), system 10 continuously and/or semi-continuously updates overlay 3251 by increasing the opacity of overlay 3251 (and/or continuously modifies another graphical parameter accordingly). In some embodiments, a target opacity (and/or other graphical parameter target) is provided by system 10 to indicate when a series of energy deliveries has reached a target (e.g. a continuously updated opacity or other graphical parameter can be compared to the target by an operator of system 10).

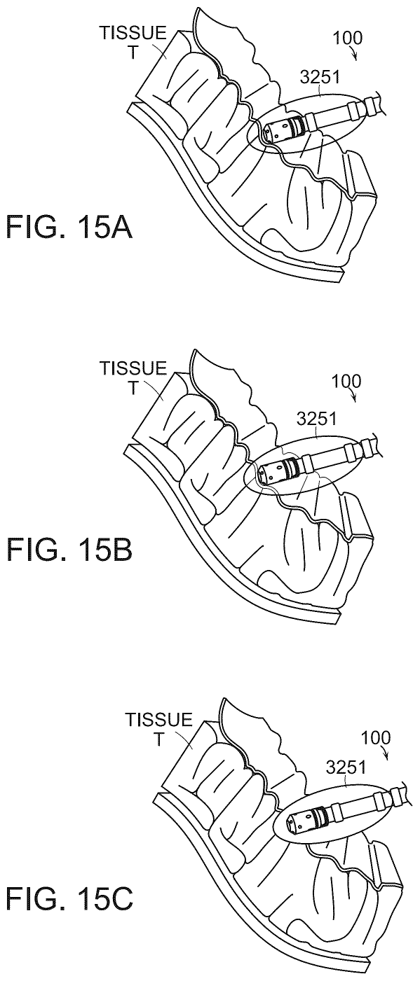

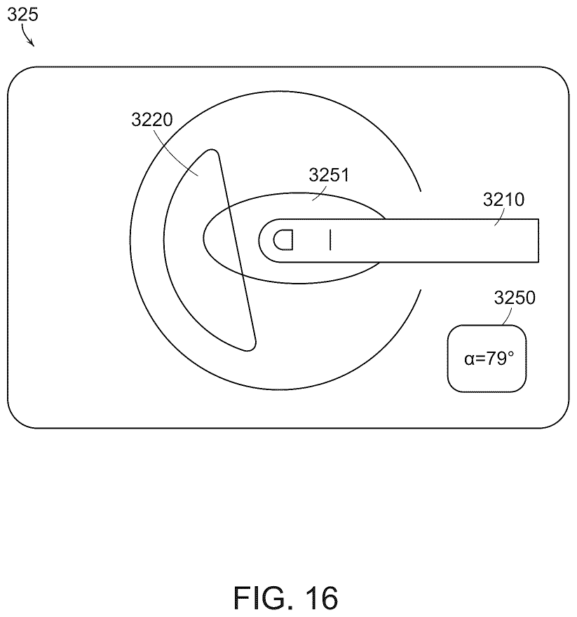

Referring now to FIG. 16, a user's view of a portion of an embodiment of a graphical user interface displaying information related to the angle of orientation between an energy delivery device and tissue is illustrated, consistent with the present inventive concepts. GUI 325 of FIG. 16 can be similar to GUI 325 described in reference to FIG. 1 and otherwise herein. GUI 325 can be configured to provide information related to orientation angle α described herein, such as is provided by orientation angle representation 3250 shown. GUI 325 can display current (e.g. real time) position information related to EDD 100 (e.g. related to distal portion 102 of EDD 100). In some embodiments, GUI 325 updates this position information, and/or any other information recorded by and/or determined by system 10 (e.g. as described herein), at least once every 30 seconds, such as at least once every 15 seconds, 10 seconds, 5 seconds, 2 seconds, or 1 second. GUI 325 can include catheter representation 3210 and tissue representation 3220 such as to provide information representing the position of a tissue surface proximate the distal portion 102 of EDD 100. In some embodiments, GUI 325 includes overlay 3251, which can be positioned relative to catheter representation 3210, as shown. In some embodiments, GUI 325 enables the user to position EDD 100 relative to tissue such that a desired portion of target tissue is positioned within the Predicted PFA Zone, such that the desired portion of target tissue is treated when one, two, three or more doses DOE are delivered.

Referring now to FIG. 17, a perspective view of embodiments of two energy delivery devices positioned with a vessel and proximate a sectional portion of tissue is illustrated, consistent with the present inventive concepts. A first energy delivery device, EDD 100a positioned within a heart chamber, is shown with its distal end positioned proximate tissue T, and a second energy delivery device, EDD 100b, is shown positioned within vessel V (e.g. an epicardial vein or artery), with its distal end positioned proximate the distal end of EDD 100a. An IEP can be delivered between energy delivery element 130a of EDD 100a and energy delivery element 130b of EDD 100b, such that the resultant electric field is produced transmurally through tissue T, as shown, such as to irreversibly electroporate the tissue (e.g. the target tissue) within the electric field. In some embodiments, EDD 100a comprises two or more energy delivery devices that are positioned in a heart chamber, and an IEP can be delivered through at least one energy delivery element 130 of each heart chamber-positioned device. Alternatively or additionally, EDD 100b comprises two or more energy delivery devices that are positioned in one or more epicardial vessels (e.g. one or more epicardial veins and/or one or more epicardial arteries), and an IEP can be delivered through at least one energy delivery element 130 of each epicardial vessel-positioned device.

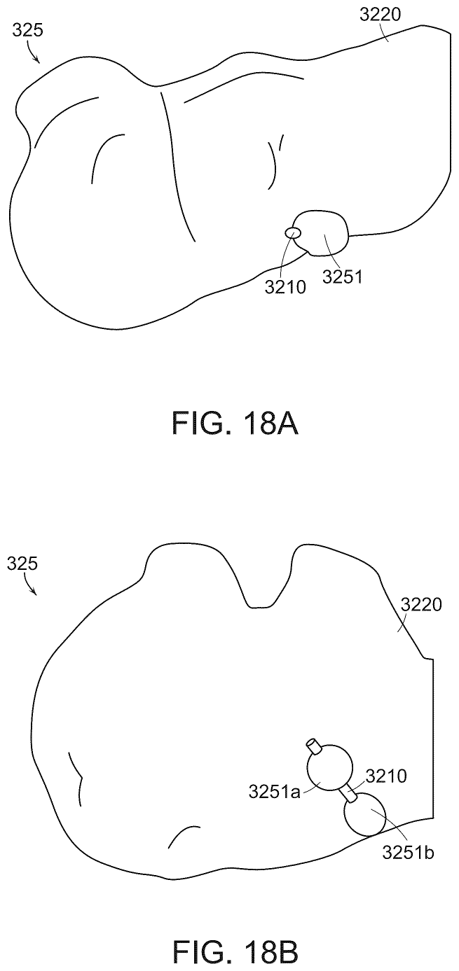

Referring now to FIGS. 18A and 18B, a user's view of a portion of an embodiment of a graphical user interface displaying an anatomic model are illustrated, consistent with the present inventive concepts. GUI 325 of FIG. 18 can be similar to GUI 325 described in reference to FIG. 1 and otherwise herein. GUI 325 can include tissue representation 3220 shown, which can comprise a computer-generated anatomic model of at least a portion of the heart (e.g. an atrium of the heart for which atrial tissue is to be treated by system 10). In some embodiments, tissue representation 3220 comprises a shell representing the interior surface of a heart chamber (e.g. a shell with zero thickness). Alternatively or additionally, tissue representation 3220 can comprise a shell with at least a portion of the shell comprising a non-zero thickness, such as a thickness representing a measured and/or an estimated thickness of the myocardium (e.g. a thickness of at least 1 mm, 2 mm, and/or 3 mm).

GUI 325 can include a representation of EDD 100, catheter representation 3210 shown, which can be displayed relative to tissue representation 3220. The position of EDD 100 relative to the tissue can be determined by localization methods of system 10, such as those described herein. GUI 325 can comprise one or more overlays 3251, such as one or more overlays 3251 as described herein. Overlay 3251 can indicate a Predicted PFA Zone and/or a VDS (e.g. a Predicted VDS or an Actual VDS), either referred to as an “energy delivery volume” herein. Overlay 3251 can comprise a 2D representation of a 3D volume. In some embodiments, portions of the volume that extend beyond the shell of tissue representation 3220 (e.g. portions of the volume that include target tissue) can be represented by a different graphical property, such as a different color, than portions of the volume within the shell (e.g. portions of portions of volume that are within the heart chamber). In some embodiments, overlay 3251 comprises multiple overlays, for example overlays 3251a and 3251b shown in FIG. 18B. Overlays 3251a,b can represent different portions of an energy delivery volume (e.g. a Predicted VDS comprising two or more discrete volumes). Overlay 3251 can comprise complex geometries, such as complex geometries representing energy delivery volumes that are based on complex electric fields. For example, overlay 3251 can comprise a “peanut” shape, representing a peanut-shaped energy delivery volume.

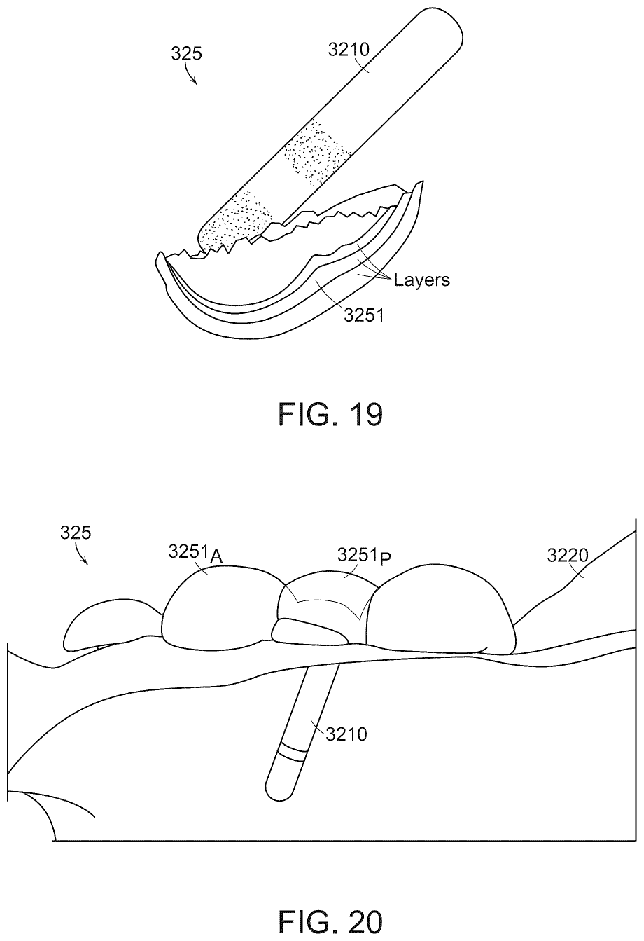

Referring additionally to FIG. 19, a portion of an embodiment of a graphical user interface displaying an overlay is illustrated, consistent with the present inventive concepts. In FIG. 19, tissue representation 3220 is not shown, for illustrative clarity. In some embodiments, only the portion of an energy delivery volume that is outside of the shell of tissue representation 3220 is represented by an overlay 3251, as shown. In these embodiments, overlay 3251 indicates to the user the volume of myocardial tissue that is located within the energy delivery zone. In some embodiments, overlay 3251 can comprise multiple “layers” indicating variation in the energy delivery volume (e.g. the VDS score throughout the volume). In some embodiments, one or more graphical properties of overlay 3251 can vary, such as to indicate the depth into myocardial tissue that the overlay currently represents. For example, if a VDS score is above a threshold depth into tissue, a portion of overlay 3251 that is located beyond the threshold can be graphically differentiated to indicate the threshold has been achieved.

Referring now to FIG. 20, a portion of an embodiment of a graphical user interface displaying predicted and actual treatment data is illustrated, consistent with the present inventive concepts. GUI 325 of FIG. 20 can be similar to GUI 325 described in reference to FIG. 1 and otherwise herein. GUI 325 can include tissue representation 3220 comprising a computer-generated anatomic model of at least a portion of the heart (e.g. an atrium of the heart to be treated by system 10), and GUI 325 can include a representation of EDD 100, catheter representation 3210, which can be displayed relative to tissue representation 3220 as shown. GUI 325 can comprise overlay 3251A representing actual energy delivery information and overlay 3251_{P} representing predicted energy delivery information. For example, overlays 3251_{A} and 3251_{P} can represent Actual VDSs and Predicted VDSs, respectively. In some embodiments, after therapy is delivered (e.g. energy is delivered) at a treatment location, an overlay 3251p representing the Predicted VDS can change color (e.g. change to an overlay 3251_{A}) to represent the Actual VDS associated with the therapy delivery. In some embodiments, as additional therapy (e.g. additional energy) is delivered proximate that treatment location, overlay 3251_{A} represents the current Actual VDS associated with the multiple therapy deliveries proximate that treatment location. In some embodiments, information displayed in FIG. 20 can be used by the user (e.g. a clinician) to improve the current Actual VDS value. For example, the user can position overlay 3251_{P} (e.g. by manipulating EDD 100) such that the resultant Predicted VDS indicates treatment delivered in that configuration would improve the current Actual VDS.

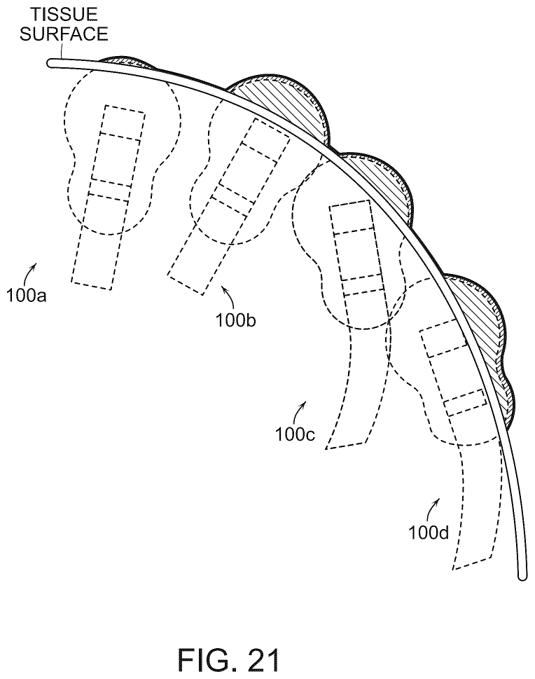

Referring additionally to FIG. 21, a schematic view of an embodiment of various treatment steps is illustrated, consistent with the present inventive concepts. Various treatment orientations of EDD 100 relative to a tissue surface (e.g. the myocardial surface) are shown. EDD 100a is shown oriented approximately 90° to the tissue surface, but not in contact with the tissue. The energy delivery volume surrounding EDD 100a is shown, only partially intersecting the tissue. EDD 100b is shown oriented approximately 90° to the tissue surface, with the tip of EDD 100b in contact with the tissue surface. The energy delivery volume surrounding EDD 100b is shown, with more of the volume intersecting the tissue (i.e. more than the volume surrounding EDD 100a). EDD 100c is shown oriented at an angle relative to tissue (e.g. approximately) 45°, with the tip of EDD 100c in contact with the tissue surface. The energy delivery volume surrounding EDD 100c is also shown. Finally, EDD 100d is shown oriented along the tissue surface (e.g. partially laying on the tissue surface). The energy delivery volume surrounding EDD 100d is shown, with the largest percentage of the zone intersecting the tissue (i.e. the largest of the four embodiments shown).

In some embodiments, data related to one or more therapeutic energy deliveries can be recorded by system 10 (e.g. recorded as Actual VDS data). As energy is delivered throughout a volume (e.g. throughout a heart chamber, including into the myocardium), system 10 can calculate the predicted effectiveness (e.g. a VDS) of the treatment throughout the volume (e.g. high, medium, and/or low probability of effectiveness). The predicted effectiveness can be displayed to the user, such as via GUI 325. The display can utilize various display properties (e.g. colors, shapes, hatch, textures, and the like) to indicate different levels of effectiveness and/or other parameters to the user. In some embodiments, the predicted effectiveness levels are differentially displayed, based on one or more thresholds of effectiveness. In some embodiments, the predicted effectiveness for a particular volume or portion of a volume can be reassessed throughout a procedure (e.g. continually assessed as additional energy is delivered, such as at least once every 5 seconds, 2 seconds, and/or 1 second). System 10 can be configured to assess the depth into myocardial tissue of a treated volume and/or the width across the surface of the tissue (e.g. the width of the cross section of the volume at the tissue surface).

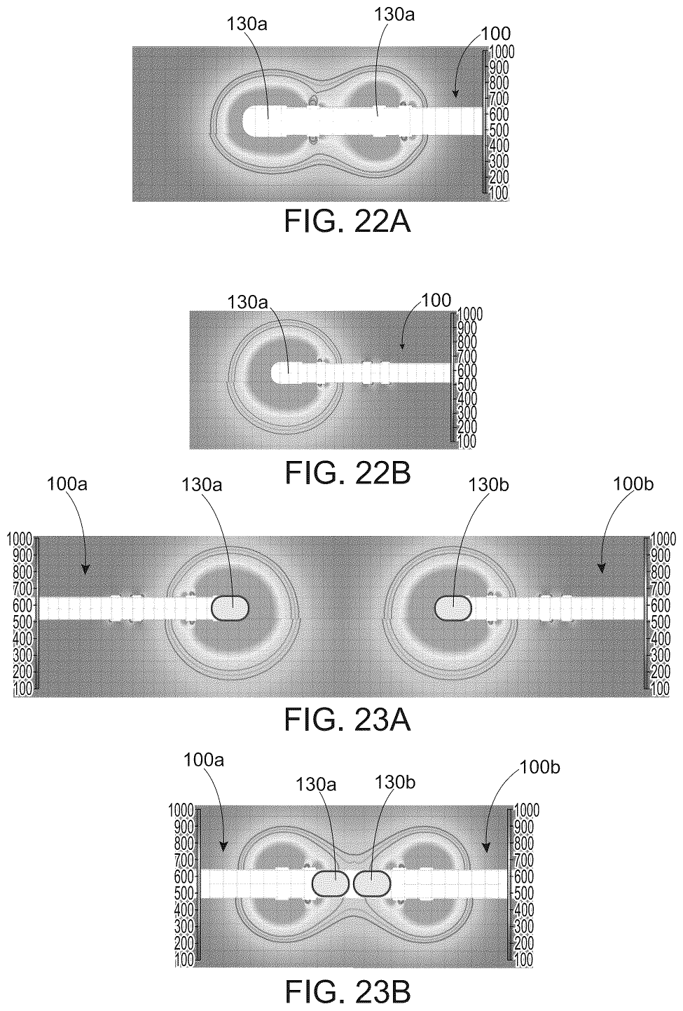

Referring now to FIGS. 22A-B and 23A-B, finite element models of various embodiments of electrode configurations are illustrated, consistent with the present inventive concepts. FIG. 22A shows a finite element model of a bipolar electrode configuration between a tip electrode and a ring electrode of an ablation catheter (e.g. electrode 130a and electrode 130b of EDD 100). FIG. 22B shows a finite element model of a unipolar electrode configuration of a tip electrode of an ablation catheter and a dispersive electrode (e.g. tip electrode 130a of EDD 100 and a return electrode, such as return electrode 130′, not shown). FIGS. 23A and 23B show finite element models of two catheters in a “kissing catheter” configuration (e.g. EDD 100a and 100b), as electrodes 130a and 130b are moved into proximity of each other such that the modeled electric fields interact with each other, as shown in FIG. 23B.

As described herein, pulse field ablation (PFA) or electroporation comprises exposing tissue to a high potential electric field causes irreversible cell death, via a metabolically mediated mechanism, and can be used to treat various arrhythmias (e.g. AF) of a patient.

Cardiac myocytes are susceptible to PFA at a threshold electric field of approximately 400-500 V/cm. The electric field generated by system 10 can be generated in a bipolar electrode configuration by applying a high voltage potential between an electrode (e.g. at the tip of a catheter, such as tip electrode 130a of EDD 100 configured as a cathode) and a return path (e.g. ring electrode 130b configured as an anode and located a fixed distance from the cathode). This bi-pole electrode distance is typically on the order of a few centimeters.

Strong electric fields suitable for PFA can also be generated by system 10 in a unipolar configuration, such as when an electrode (e.g. electrode 130a) at the distal tip of EDD 100 is configured as a cathode, and a large dispersive electrode (e.g. one or more electrodes of a patch 60) is configured as the anode. The large dispersive electrode can be located on a patient's back or sternum. In unipolar configurations, as the return path is distributed over a large area and is far removed from the cathode, the high potential electric field is limited to the volume relatively proximate the tip electrode of EDD 100.

In some embodiments, a second catheter is used to provide a return path (e.g. an electrode 130 of the second catheter is configured as an anode). The second catheter can comprise another ablation catheter, a sheath (e.g. with distal electrodes), and/or a multi-polar fixed or steerable catheter. The second catheter can be configured to be positioned in the coronary vasculature, another chamber of the heart, and/or in the pericardial space. In this type of configuration, since the distance between electrodes is not fixed and may be adjusted as needed by the physician, the electric field contours (and lesion volume) are a function of the positions of the associated devices (e.g. the positions of the associated electrodes).

Using system 10 of the present inventive concepts, the position of multiple catheters can be determined, such as with impedance-based and/or magnetic-based localization methods, such as are described herein. Using these calculated positions, the electric field contours can be calculated in real time and/or near real time (“real time” herein). The electric field contours can be displayed on one or more screens for the user (e.g. a clinician) to plan a therapeutic treatment. The position of the catheters can also be used to dynamically adjust the voltage applied to the cathode-anode pair as a means to control the electric field shape and lesion depth. In some embodiments, system 10 is configured to use positional data of the catheters to adjust voltage levels, such as to avoid flash arcing which can result at very high electric field potentials, and can lead to dielectric breakdown and plasma formation.

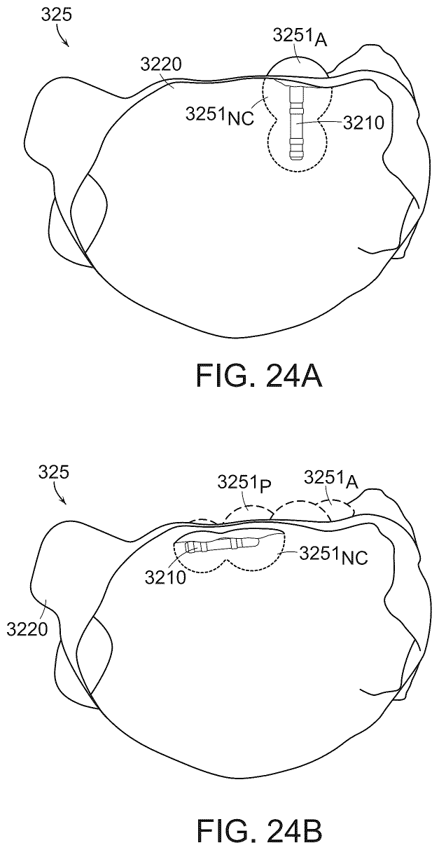

Referring now to FIGS. 24A and 24B, a user's view of a portion of an embodiment of a graphical user interface displaying an anatomic model are illustrated, consistent with the present inventive concepts. GUI 325 of FIGS. 24A and 24B can be similar to GUI 325 described in reference to FIG. 1 and otherwise herein. GUI 325 can include tissue representation 3220 comprising a computer-generated anatomic model of at least a portion of the heart (e.g. an atrium of the heart to be treated by system 10), and GUI 325 can include a representation of EDD 100, catheter representation 3210, which can be displayed relative to tissue representation 3220 as shown. GUI 325 can comprise overlay 3251_{A} representing actual energy delivery information and overlay 3251_{P} representing predicted energy delivery information. For example, overlays 3251_{A} and 3251_{P} can represent Actual VDSs and Predicted VDSs, respectively. In some embodiments, after therapy is delivered (e.g. after energy is delivered), an overlay 3251p representing a Predicted VDS can change color to represent an Actual VDS (e.g. change to an overlay 3251_{A}). In some embodiments, GUI 325 can comprise overlay 3251_{NC} representing an area surrounding EDD 100 where the predicted electric field is at a sufficient level enough to affect target tissue present within the field (e.g. cardiac wall tissue within the field), however no target tissue is present within that portion of the predicted electric field.

In FIG. 24A, EDD 100 (e.g. represented by catheter representation 3210) is positioned axially relative to (e.g. orthogonal to) a portion of the cardiac wall. Overlay 3251_{A} indicates an Actual VDS relating to therapy applied from the orientation shown. In FIG. 24B, EDD 100 is positioned lateral to (e.g. parallel to) a portion of the cardiac wall, proximate portions of tissue where therapy has previously been applied. Overlay 3251_{P} represents a Predicted VDS relating to therapy to be applied from EDD 100 in the orientation shown. Overlay 3251_{A} represents Actual VDS where therapy has previously been applied.

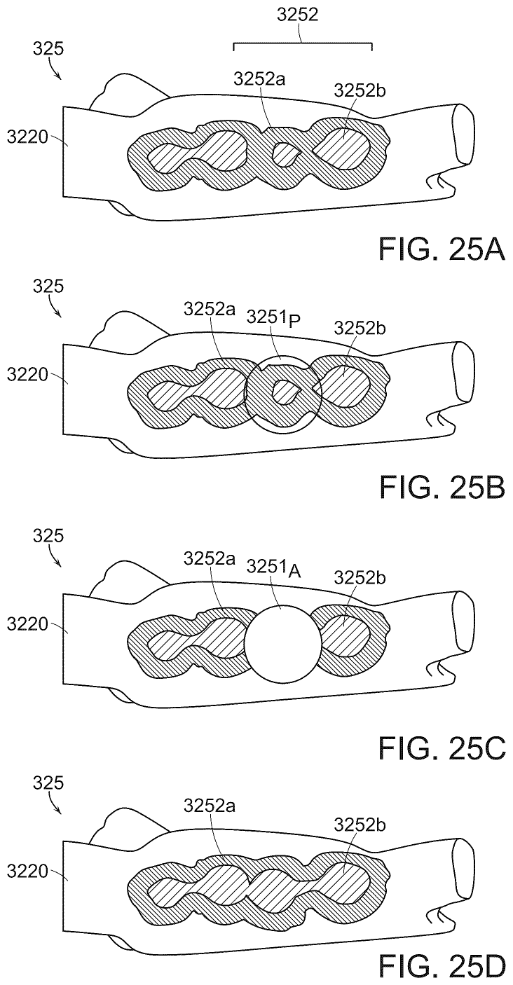

Referring now to FIGS. 25A-D, a user's view of a portion of an embodiment of a graphical user interface displaying data relative to a treatment threshold are illustrated, consistent with the present inventive concepts. GUI 325 of FIGS. 25A-D can be similar to GUI 325 described in reference to FIG. 1 and otherwise herein. GUI 325 can include tissue representation 3220 comprising a computer-generated anatomic model of at least a portion of the heart (e.g. an atrium of the heart to be treated by system 10). GUI 325 can include one or more overlays indicating locations where therapy (e.g. energy) has been applied to target tissue, overlay 3252 shown. Overlay 3252 can include a first overlay portion, overlay 3252a, indicating locations where therapy has been delivered to target tissue, at a depth under a threshold (e.g. a depth of less than 3 mm, and/or less than transmural). Overlay 3252 can include a second overlay portion, overlay 3252b, indicating locations where therapy has been delivered to target tissue at a depth equal to and/or above a threshold (e.g. a depth of at least 3 mm, and/or transmurally).

In treating a patient, it can be desirable to create a contiguous therapeutic lesion by delivering energy to tissue using system 10 (e.g. energy delivered via EDD 100). To maximize the therapeutic benefit, the contiguous therapeutic lesion can comprise at least a threshold depth (e.g. a minimum depth to sufficiently achieve a desired efficacy), such as a depth of at least 3 mm. Alternatively or additionally, the lesion can be delivered transmurally (e.g. throughout the full thickness of the cardiac wall) to maximize the therapeutic benefit (e.g. sufficiently achieve a desired efficacy). FIG. 25A shows overlay 3252b indicating a non-contiguous lesion with a thickness above a threshold. FIG. 25B includes overlay 3251p (described herein), representing therapy to be delivered (e.g. energy delivered via EDD 100, not shown), such as to “fill in” (e.g. sufficiently treat) the non-contiguous areas of the lesion of FIG. 25A (e.g. “fill in the gaps”). FIG. 25C shows overlay 3251_{A} (also described herein), representing therapy having been delivered. FIG. 25D shows a contiguous lesion above a threshold depth (e.g. contiguous overlay 3252b), indicating the therapy delivered as indicated in FIG. 25C successfully “filled in” the gaps of the therapeutic lesion.

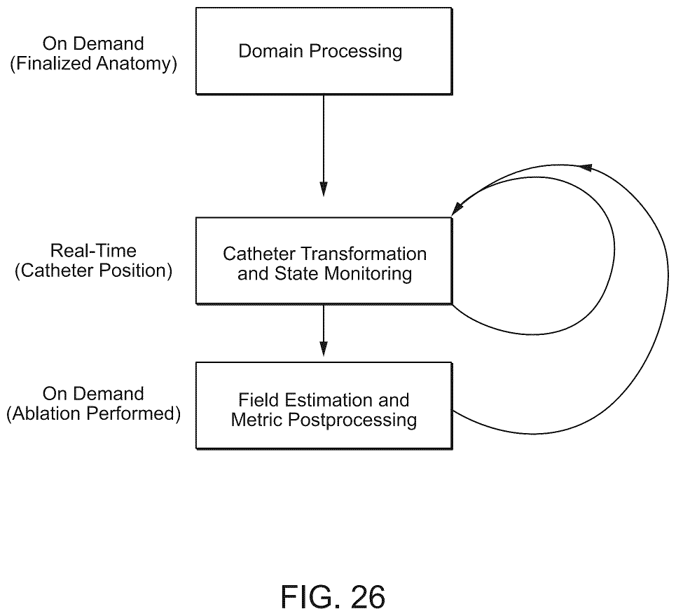

Referring now to FIG. 26, a flow chart showing a general workflow of algorithms for performing field tagging is illustrated, consistent with the present inventive concepts. In some embodiments, system 10 includes one or more “Field Tagging algorithms” (such as algorithm 335 described in reference to FIG. 1 and otherwise herein), such as one or more algorithms that operate in various regimes, such as regimes referred to herein as “Domain Preprocessing”, “Catheter Transformation and State Monitoring”, and “Field Estimation and Metric Postprocessing”.

A domain preprocessing algorithm can refer to one or more algorithms of system 10 that define various operations to be performed on the anatomy and/or the surrounding space, and typically relate to algorithms that produce a discretized structure by which spatial operators can be computed. As shown in FIG. 26, domain preprocessing can be performed by system 10 before subsequent algorithms are determined and/or utilized. Domain discretization can be fundamental to any computational model, and it can include the process of taking a spatial continuum (such as an anatomy) and subdividing it, such as for the purpose of measurement, analysis, and/or visualization. Anatomical surface discretization can be performed by system 10, and the discretization can be used by system 10 to produce, for example, a simple surface constrained, triangular element-based, discretization. In some embodiments, a field tagging operation comprises a volumetric operation and therefore requires volumetric discretization. This volumetric discretization can take many forms, for example, a rasterized regular grid (e.g. a grid comprising hexahedral elements), or alternatively an unstructured tessellation of tetrahedral elements. The discretized volume, herein referred to as a volumetric mesh, can be constructed to extend beyond the anatomy (e.g. in some or all dimensions) and can comprise a predefined density that can vary depending on the application. Once the volumetric mesh has been populated and/or scaled to the appropriate dimensions, system 10 can determine what tissues of each of the elements of the mesh lie within a particular location and/or a typical type of tissue (e.g. locations present in the blood, myocardium, and/or pericardium), such that system 10 can associate tissue-dependent properties with each element.

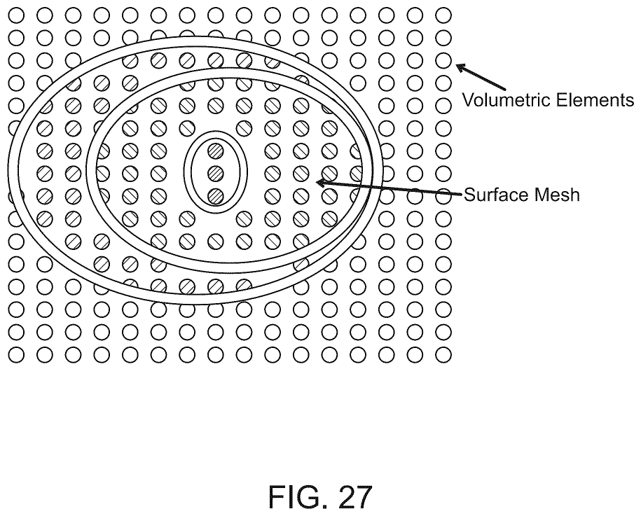

Referring additionally to FIG. 27, a graphical representation of domain delineation is illustrated, consistent with the present inventive concepts. After system 10 has populated the computational domain with the volumetric mesh, system 10 can then delineate the various tissue types, such that the field calculations can be augmented and analytics can be performed based on tissues determined to be present. This tissue delineation can be performed by system 10 using a signed distance operation in which a surface-based mesh (e.g. a “water-tight” mesh) is used to distinguish which portion(s) of the domain are situated within the anatomical structure, and which portion(s) are outside of the anatomical structure. In some embodiments, tissue delineation can be stratified based on several surface meshes, for example as indicated in the schematic shown in FIG. 27. These surfaces can have any configuration relative to one another (e.g. concentric, mutually exclusive, and/or non-mutually exclusive). This process can be applied by system 10 to any number of surfaces and can be performed across level set solutions of the original anatomical surface. As a result of this operation, system 10 can determine which elements of the volumetric mesh are within each surface, as well as the geodesic distance of each element away from each surface in the domain. These data layers (within which tissues and distance away from tissues are stored by system 10) are fundamental to the operations performed in the transformation and field estimation portions of the workflow (shown in FIG. 26). In some embodiments, system 10 is configured to adapt how a digital representation of a catheter (e.g. catheter representation 3210 not shown but described herein) “snaps” to the surface (or not), based on tissue location and/or tissue type to which the catheter is near, based on the delineated domain.

Once the domain has been populated and delineated, system 10 can be configured to visualize the navigation of the catheter within the heart (e.g. to determine the location of a catheter within the heart and display a representation of the catheter to a user relative to the anatomy), a function referred to herein as “Catheter Transformation and State Monitoring”. This portion of the workflow provides real-time updates of catheter position and transforms the associated primitive geometries (for example, a catheter can comprise a digital representation including an a priori eikonal solution, and/or a sheath or additional hardware can comprise a primitive digital representation). As used herein, the representation of “a catheter” and/or “the catheter” can refer to any one, two, or more catheters or other inserted devices that are localized and displayed to the user (e.g. an ablation catheter, a sheath, a mapping catheter, and/or other device). To perform this transformation, system 10 can determine a transformation matrix between the default catheter position and the new catheter position. This determination can be performed by solving for the rigid rotational matrix between the live coordinates and the coordinates of the catheter in its default position. This transformation can be performed using Euler angles and/or quaternion-based solutions. Solving for the rotation matrix can provide system 10 with the position, orientation, and/or polarity of the catheter and/or associated primitive fields. For any position of the catheter, system 10 can determine the domain (e.g. the tissue type) to which the catheter is currently located, and/or the distance that the catheter is from a surface (e.g. any surface).

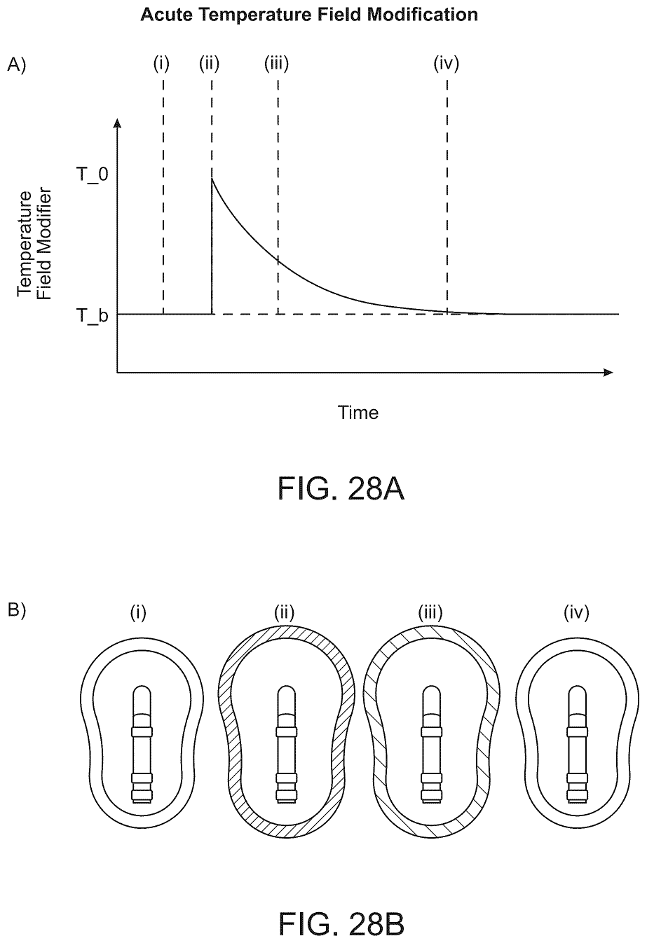

Referring additionally to FIGS. 28A and 28B, a graph of a measured catheter parameter and various representations of the catheter are illustrated, consistent with the present inventive concepts. System 10 can use information recorded and/or determined (e.g. calculated) that relate to a catheter, to drive state changes in the catheter model (e.g. changes in the visual representation of the model). In some embodiments, the state changes are based on the positioning of the catheter alone. For example, system 10 can be configured to change the color and/or another visualization feature when the catheter is within a threshold distance of (e.g. less than 1 mm away from) a target location (e.g. a portion of the endocardial surface of a heart chamber). Additionally or alternatively, other parameters, such as force, can be used to drive these state changes. In some embodiments, the parameters used by system 10 are not strictly geometric. State changes can be elicited (e.g. and determined by system 10) based on changes to: local tissue impedance change, global impedance change, local induced temperature changes, force magnitude, and/or other biophysical quantities. The state change can be a permanent change and/or a change tied to a transient phenomenon (such as induced temperature changes). For example, as shown in FIGS. 28A and 28B, a transient change is illustrated, where the size of a predicted therapy is augmented based on a change to the computational domain. These state changes can also relate to the mode of operation employed in the “Field Estimation” phase of the workflow, based on the local environment. In some embodiments, an a priori eikonal solution can be computed by system 10 in real time and/or it can be computed ahead of time depending on computational resources. The state changes can be based on the chosen energy delivery parameters (e.g. power level, electrode configuration, pulse train pattern, and/or other PFA parameter).

A final portion of the field tagging workflow, the “Field Estimation and Postprocessing” can be initiated by system 10 when an ablation is performed, and it can contain the majority of the novel contributions. During ablation, the field can be estimated (e.g. in one or more of a number of ways) to allow for field tagging to be functional (e.g. in a number of operating conditions). For example, in cases where a volumetric raster cannot be completed, algorithm 335 can run in a strictly primitive capacity, for example, where the interaction between the eikonal primitive and the anatomical shell is computed as compared to the eikonal solution. Additionally or alternatively, system 10 can perform a process that utilizes local anatomical features and the orientation of a catheter relative to the anatomical shell to choose from a number of prior computed solutions that are mapped and/or projected onto the anatomical surface.

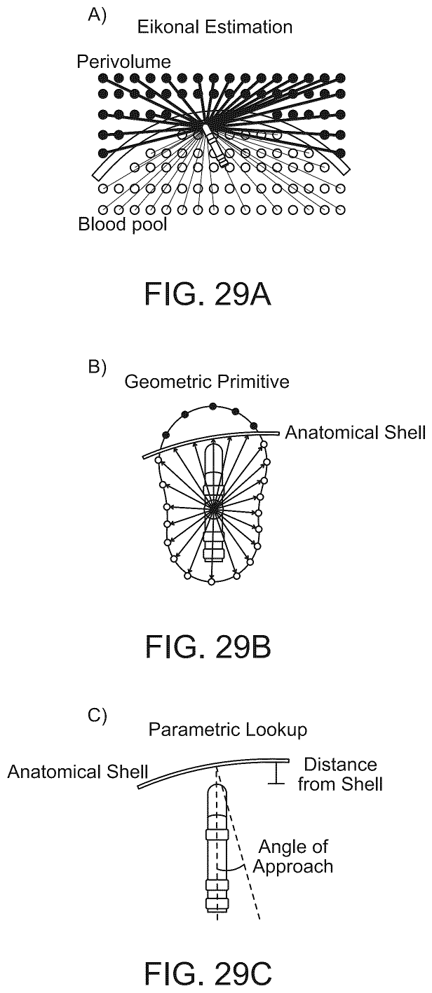

Referring additionally to FIGS. 29A-C, various techniques of field estimation are illustrated, consistent with the present inventive concepts. Each method can utilize the underlying grid to parameterize the solution to the electric field simulation. In FIG. 29A, an eikonal estimation is illustrated. In FIG. 29B, a geometric primitive estimation is illustrated. In FIG. 29C a parametric lookup method is illustrated.

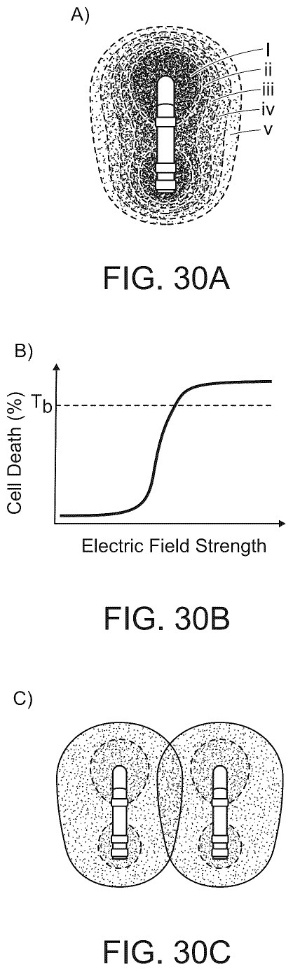

Referring additionally to FIGS. 30A-C, a representation of a multi-level attrition profile allowing for dynamic updates to applied therapy is illustrated, consistent with the present inventive concepts. During each ablation, the volumetric electric field can be estimated by system 10 and used to evaluate a therapeutic threshold and/or a volumetric attrition profile. Monitoring this information by system 10 allows for regions of tissue that are within a subtherapeutic range of EDD 100 (e.g. a catheter configured to deliver pulse field ablation and/or other energy) after one application (e.g. one energy delivery), but that can be treated with subsequent applications (e.g. subsequent energy delivery performed over a time period of seconds, minutes, and/or hours), to be identified (e.g. tracked) by system 10. In some embodiments, the estimation of therapy (e.g. efficacy of therapy) determined by system 10 can be adapted dynamically. An attrition profile produced by system 10 can be driven by an underlying function, as shown in FIGS. 30A-C in which the relationship between an applied field (such as an electric field) and cell death is illustrated. Cell death can be a function of electric field (e.g. electric field strength), distance from a given electrode, and/or distance from a field primitive (e.g. an a priori geometric template that describes the electric field). FIG. 30C shows how two subtherapeutic regions can be superimposed to create a supratherapeutic region. These thresholds can similarly be augmented by temperature, force, anatomical proximity, changes due to local anatomical features, local/global impedance, and/or other biophysical quantities.

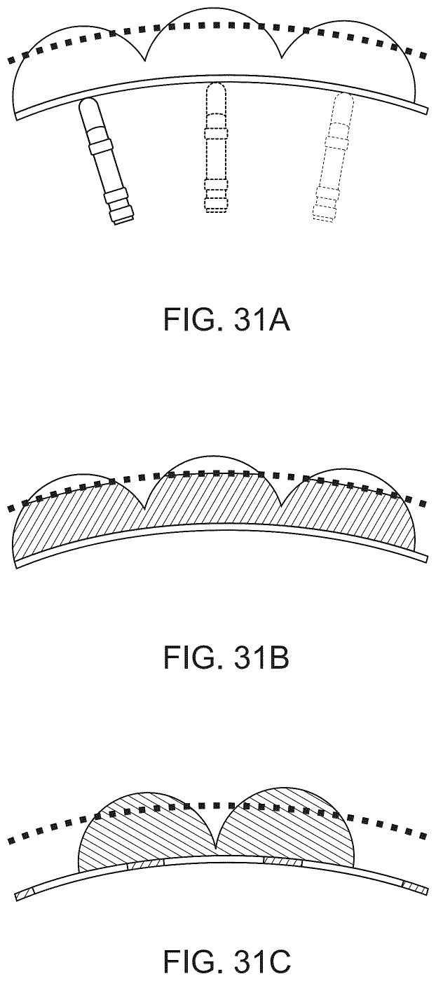

Referring additionally to FIGS. 31A-C, examples of a depth projection feature are illustrated, consistent with the present inventive concepts. After system 10 calculates an estimation of a field, the subsequent suprathreshold tissue can be estimated, visualized, and/or utilized for subsequent postprocessing by system 10. Upon each energy delivery to tissue (e.g. via delivery of an applied electric field), the depth of the therapeutic dose of the electric field can be determined and projected onto a shell of the anatomy by system 10. This projection can be performed using anatomical surface normals, using the surface normal of the isosurface of therapy (via a nearest neighbor projection), and/or using a compound level-set type projection. This projection can be used to estimate the projection distance, and therefore, the depth of the desired therapy. This projection can be on the anatomical shell, on an offset version of the anatomy, and/or on another shell otherwise determined by system 10 (e.g. using imaging information and/or atlas-based information). After and/or during each energy delivery (e.g. creation of each lesion), the projection can be refactored such that the maximal depth is stored on an element-by-element (or node-by-node) basis. This visualization can be in a banded, binarized, and/or continuous form. From these depth projections, system 10 can estimate lesion gaps both superficially as well as below the endocardial surface. Such a prediction is shown in FIG. 31C with the lesions shown in hatch pattern. The depth at which the binarized threshold and the subsequent contiguity at depth assessments can be assessed, can be set at a user-specified threshold and/or a system 10 determined threshold. This threshold can be an offset from an anatomical surface or other anatomical location (e.g. a level-set of the anatomy), the offset can be atlas-based, and/or the offset can be patient specific (e.g. as if determined by system 10 based on imaging data).

In some embodiments, a lesion prediction (e.g. a lesion quality and/or efficacy prediction) can be prospective and/or retrospective. In a retrospective state, all pairs of lesions within a configurable distance threshold can be assessed by system 10, such as an assessment for contiguity both superficially and/or at depth. Lesions determined to have intramural gaps (e.g. depth gaps) and/or epicardial gaps (e.g. surface gaps), for example as identified in the depth projection and/or via the volumetric mesh, can be visually highlighted, and/or gap-filling targets can be identified and/or provided by system 10 (such as is shown in FIGS. 30A-C).

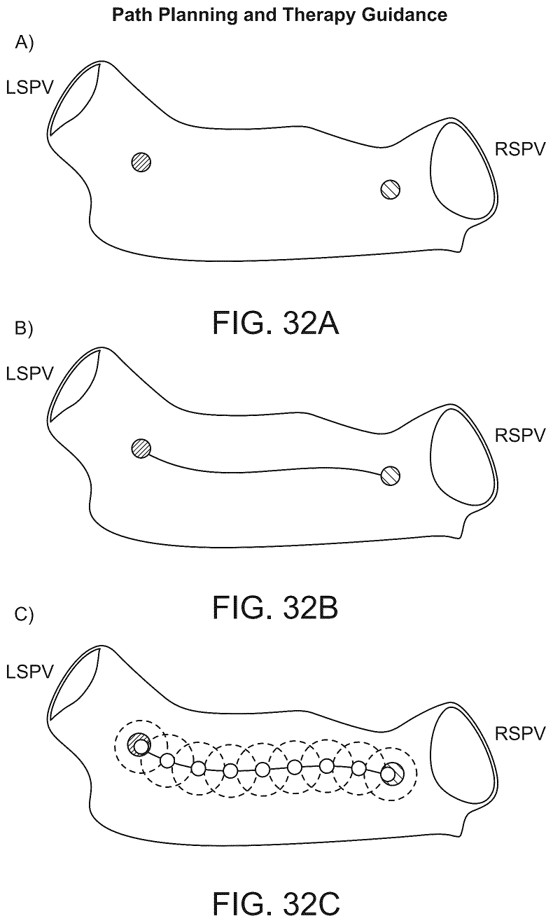

Referring additionally to FIGS. 32A-C, an example of a method of prospective lesion set planning is illustrated, consistent with the present inventive concepts. In some embodiments, a user can place two, three, or more points on a surface, as shown in FIG. 32A, and the shortest surface-based distance (e.g. geodesic and/or Euclidean distance) can be computed by system 10 across an anatomical surface (or across an analogue surface, such as an offset), as shown in FIG. 32B. In some embodiments, an optimized lesion placement can be estimated by system 10 and displayed, as shown in FIG. 32C. In some embodiments, an inter-lesion gap criteria can be used by system 10, such as to change the state of the real-time eikonal primitive (for example, to change it from a default color to a different color when in a position that would fill a gap). In some embodiments, these path and lesion estimations can depend on the user selected contiguity at depth parameter, which can use a user specified depth at which the algorithm estimates depth. Alternatively or additionally, the estimations can depend on atlas-based thickness information and/or patient specific thickness information.

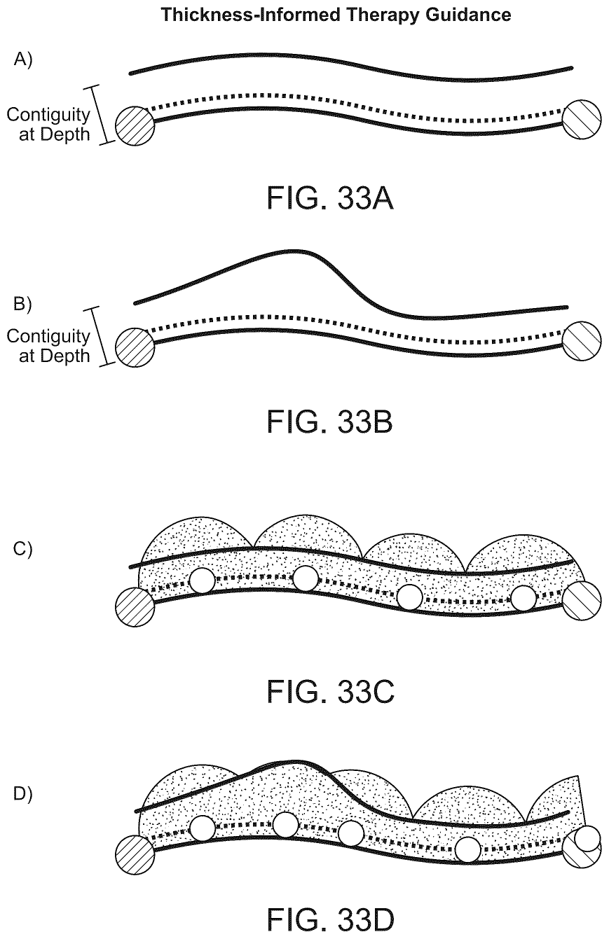

Referring additionally to FIGS. 33A-D, an example of a thickness-based therapy planning is illustrated, consistent with the present inventive concepts. Predictions provided by system 10 can be adapted (e.g. in real time) based on changes to the EDD 100 (e.g. a pulsed field ablation catheter) configuration, and/or a domain in which the delivered electric field is being estimated by system 10. In some embodiments, these lesion sets determined by system 10 can be selected by dropping points, as indicated above, or the lesion sets can be selected from a group of common lesion set approaches (e.g. as stored by system 10). If a lesion set is chosen, the same prediction can be performed by system 10 on the automatically identified lesion set, for example, when the prediction is based on the anatomy of the heart (e.g. the anatomy of the atria). A universal atrial coordinate (UAC) system of system 10 allows for anatomical inferences to be calculated by system 10, for example, the inferences being based on a series of topological and/or manifold based assessments of the anatomical surface.

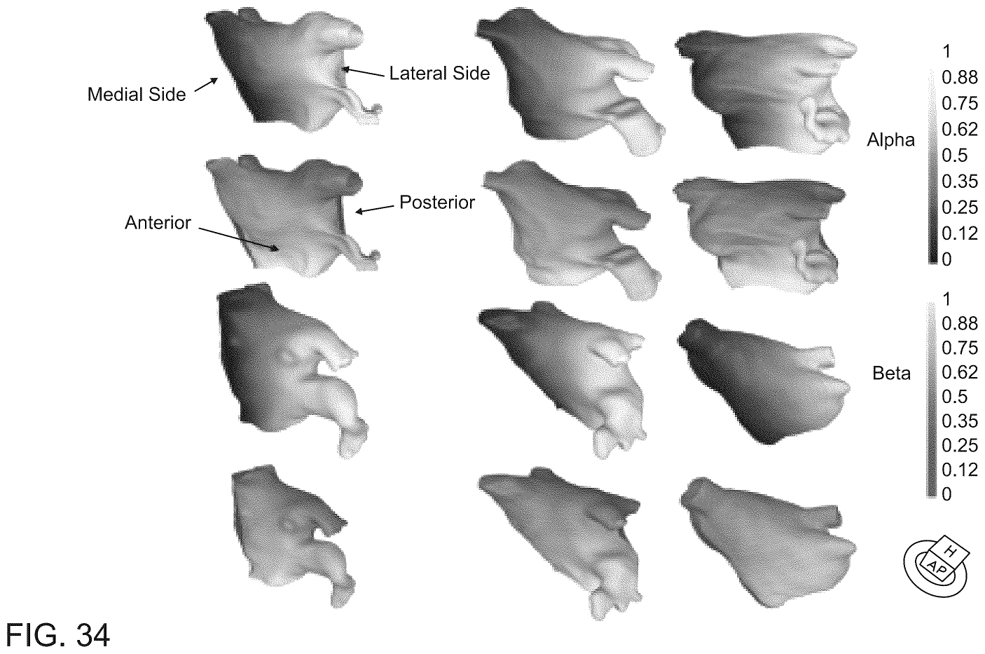

Referring additionally to FIG. 34, a UAC-based system implemented across several left atrium geometries is illustrated, consistent with the present inventive concepts. As shown, the UAC-based mapping of system 10 can be generalized to the anatomy, and system 10 can be configured to allow for anatomical mapping between geometries, such as anatomical mapping of scalar information, vector information, therapeutic information, case information, and/or tensor information.

The above-described embodiments should be understood to serve only as illustrative examples; further embodiments are envisaged. Any feature described herein in relation to any one embodiment may be used alone, or in combination with other features described, and may also be used in combination with one or more features of any other of the embodiments, or any combination of any other of the embodiments. Furthermore, equivalents and modifications not described above may also be employed without departing from the scope of the inventive concepts, which is defined in the accompanying claims.

1.-70. (canceled)

71. An energy delivery system, comprising:
one or more energy delivery devices having an energy delivery portion comprising at least one energy element configured to supply energy to target tissue; and
a processor configured to execute an algorithm to calculate a volume-distributed score (VDS) for a volume surrounding the energy-delivery portion of the one or more devices,
wherein calculated VDS represents an estimation of therapy effectiveness for a future delivery of energy and/or of therapy effectiveness of an energy delivery already performed.

72. The system of claim 71, wherein the target tissue comprises tissue selected from the group consisting of:
cardiac tissue; nerve tissue; vessel wall tissue; organ tissue; brain tissue; lung tissue; kidney tissue; liver tissue; stomach tissue; muscle tissue; and combinations thereof.

73. The system of claim 71, wherein the processor is further configured to render the VDS in a visual form on a display of a user interface.

74. The system of claim 73, wherein the visual form includes the VDS in an elongate form.

75. The system of claim 73, wherein the processor is further configured to limit the display of the VDS to include only VDS values that exceed a threshold.

76. The system of claim 75, wherein the processor is configured to set or change the threshold in response to a user input via the user interface and to visually render the variability of scoring differences on the display in different areas of the target tissue.

77. The system of claim 73, wherein the processor is further configured to display the VDS in a visual form in relation to the target tissue.

78. The system of claim 73, wherein the processor is further configured to display the VDS as at least one projection onto an anatomic model.

79. The system of claim 73, wherein the volume of space represented by the VDS can extend far enough to encompass regions that are highly probable, marginally probably, and/or minimally probable of receiving a therapeutically effective delivery of energy for irreversible electroporation.

80. The system of claim 73, wherein the processor is configured to render a spatial distribution of VDS values in the display using color, opacity, intensity, and/or another variable graphical parameter to indicate differentiation.

81. The system of claim 71, wherein the therapeutically effective delivery of energy causes an alteration of the target tissue, wherein such an alteration can comprise: an electrical alteration of tissue where cells of the tissue are stunned or killed; a biophysical alteration of tissue where the tissue is thickened, tissue is thinned, and/or edema results; a biochemical alteration of tissue; a material alteration of tissue where tissue is stiffened, softened, made porous, and/or made brittle, and/or combinations of these.

82. The system of claim 71, wherein the processor is further configured to calculate the VDS based one or more metric, comprising at least one of electrical field strength; area of contact; angle of contact; force of contact; temperature; impedance; one or more tissue properties; blood properties; and/or combinations of these.

83. The system of claim 71, wherein the processor is further configured to calculate the VDS by taking into account one or more of: a configuration of the at least one energy delivery element; a configuration of an energy waveform and/or other energy delivery parameter; and/or blood geometry; target tissue geometry; and/or properties relative to the one or more delivery devices.

84. The system of claim 71, wherein the algorithm is configured to use a field model that is based on: analytical calculations and/or equations; discrete and/or numerical methods using randomized point sources with modeled boundary conditions; and/or a look-up table and/or estimation and/or optimization algorithm with data fitting based on a priori data including at least one or a simulation, a database, and/or a training dataset.

85. The system of claim 71, wherein the algorithm includes an optimization algorithm that includes statistical models, pattern matching, machine learning, artificial intelligence, and/or other data-driven techniques based on historical data to calculate the VDS.

86. The system of claim 71, wherein the algorithm includes a vulnerability algorithm that is executable to processes a set of actual VDS values of a region to qualitatively label and/or quantitatively display areas in which delivered energy is below a predetermined effectiveness threshold.

87. The system of claim 86, wherein the vulnerability algorithm is executable to process a set of VDS values to identify data features that are deterministically and/or probabilistically ineffective.

88. The system of claim 86, wherein the vulnerability algorithm is executable to search for spatial gaps, edges, channels, and/or spatial heterogeneity in the VDS values.

89. The system of claim 86, wherein the vulnerability algorithm is executable to utilize image processing and/or pattern matching techniques to assess vulnerability.

90. The system of claim 71, wherein the at least one energy delivery element includes at least one energy delivery element disposed on an ablation catheter.
